(19) United States
(12) Patent Application Publication
(10) Pub. No.: US 2014/0249105 A1
(43) Pub. Date: Sep. 4, 2014

(54) NOVEL CERAMIDE ANALOGUES, PROCESSES FOR PREPARING SAME AND USES THEREOF

(71) Applicants: Jean-Louis Brayer, Nanteuil Le Haudouin (FR); Natacha Frison, Verneuil En Halatte (FR); Benoit Folleas, Senlis (FR)

(72) Inventors: Jean-Louis Brayer, Nanteuil Le Haudouin (FR); Natacha Frison, Verneuil En Halatte (FR); Benoit Folleas, Senlis (FR)

(73) Assignee: Diverchim, Montataire Cedex (FR)

(21) Appl. No.: 14/234,727

(22) PCT Filed: Jul. 25, 2011
(86) PCT No.: PCT/FR2012/051592

§ 371 Date: Mar. 11, 2014

(30) Foreign Application Priority Data
Jul. 25, 2011 (FR) 11/02317

(51) Int. Cl. C07C 233/41; C07C 231/02; C07F 9/40; C07C 237/16; C07F 7/18; C07C 235/28; C07D 309/12

(52) U.S. Cl. CPC C07C 233/41; C07C 235/28; C07C 231/02; C07D 309/12; C07C 237/16; C07F 7/1864; C07F 7/1892; C07F 9/4018
USPC 514/63; 564/152; 564/138; 514/616; 549/419; 514/459; 560/193; 514/548; 558/267; 514/512; 556/421; 564/15; 514/102

(57) ABSTRACT

Compounds, ceramide analogues, having a cyclic structure derived from cyclopropane, cyclobutane or cyclopentane, the ring bearing two chains consisting of an amide function. Each amide function is attached to the ring by the nitrogen atom of the function and carries a hydrocarbon chain derived from a fatty acid. The amide functions can be cis or trans relative to one another. Processes for the preparation of these novel compounds as well as pharmaceutical and/or cosmetic compositions containing them.

The invention relates to novel ceramide analogues, processes for the preparation thereof and applications thereof in pharmaceutical and cosmetic compositions.

The ceramides represent a particular class of intraepidermal lipids naturally present in the skin and hair. They are formed from sphingosine or sphingosene, which combines with certain unsaturated fatty acids such as linoleic acid. They play a fundamental role in the structure of the epidermis and its functions, in particular in maintaining and controlling the hydration and cohesion of the stratum corneum. However, the content of ceramides in the skin varies under various conditions: it is higher when the epidermis is subjected to various aggressive factors such as injury, exposure to sunlight, and increased evaporation of water from the skin, and it decreases in the elderly and in subjects with atopic dermatitis.

Ageing of the subject therefore leads to a decrease thereof in the skin as well as the appearance of brown spots. The spots are treated with depigmenting products, in particular retinoic acid, azelaic acid, ascorbic acid and hydroquinone. All of these have side-effects. In particular, hydroquinone, one of the best-known depigmenting agents, leads to degeneration of collagen and elastin fibres, and has genotoxic and carcinogenic effects. It is now only used on medical prescription.

A subject of the present invention is novel cyclic diamides in which the two amide functions are carried by a ring comprising from 3 to 5 carbon atoms, based on the constituent elements of the lipid bilayers constituting the cell membranes, and processes for the preparation thereof.

Another purpose of the invention is the development of pharmaceutical and cosmetic preparations to take advantage of their biological effects, in particular as agents for combating ageing of the skin and as depigmenting agents.

A more particular subject of the invention is the compounds represented by general formula I shown below

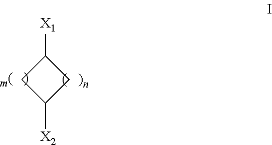

in which

m=1, 2, 3 and n=0, 1

provided that m+n is different from 4,

X₁ and X₂ can be trans or cis relative to one another and represent, independently of one another, a group selected from

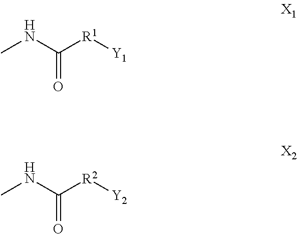

in which
[symbol] Y₁ and Y₂ represent, independently of one another,
   —H,
   —OH,
   —OH optionally coupled to a glycoside compound, which can be an α- or β-furanose or an α- or β-pyranose,
   —ORₐ,
   —OCOCH₃,
   —OSi(Rₐ)₃,
   —OSitBdPh of formula

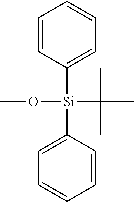 [SMILES: CO[Si](c1ccccc1)(c1ccccc1)C(C)(C)C]

—OSitBdM of formula

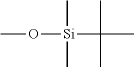

—COOH,
—COOR_{b},
—NH₂,
—NR_{c}R_{d},
—NHCORₑ,
—NHCOOR_{f},
the —OTHP group of formula

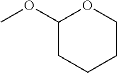

a group derived from ethylene glycol of formula

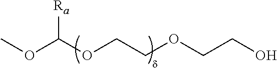

in which δ varies from 1 to 12,
a group derived from propylene glycol of formula

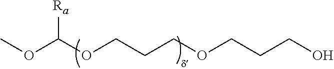

in which δ varies from 1 to 5,
an —O—CH(R_{z})—O-Q group, in which R_{z}, represents an alkyl or aralkyl group comprising from 1 to 30 carbon atoms, which can, but does not necessarily, contain one or more ether functions and optionally a terminal hydroxyl,
   Rₐ, R_{b}, R_{c}, R_{d} represent linear or branched alkyl groups comprising from 1 to 4 carbon atoms, optionally substituted by one or more halogen atoms, or carbon chains interrupted by oxygen or sulphur atoms, benzyl groups optionally substituted by a halogen atom, a hydroxy group, an alkoxy group comprising from 1 to 8 carbon atoms,
   Rₑ represents a linear or branched alkyl group containing from 1 to 4 carbon atoms, a phthalimido group (in this case the NH is replaced by N), a benzyl group optionally substituted by a halogen atom, a hydroxy group, an alkoxy group and in particular substituted by the methoxy group in the para position,
   R_{f} represents a linear or branched alkyl group comprising from 1 to 4 carbon atoms, optionally substituted by one or more halogen atoms, or a carbon chain interrupted by oxygen or sulphur atoms, a phenyl group, a benzyl group optionally substituted by a halogen atom, a hydroxy group, an alkoxy group and in particular substituted by the methoxy group in the para position,
the phosphonate group of formula

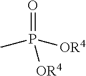

in which
R⁴ represents a linear or branched alkyl group, comprising from 1 to 6 carbon atoms, in particular methyl, ethyl, isopropyl, tert-butyl, (OR⁴)₂ optionally forming a ring between the two oxygen atoms, the (OR⁴)₂ groups in particular originating from diols such as ethane-1,2-diol, propane-1,3-diol, 2,2-dimethylpropane-1,3-diol, 2,3-dimethylbutane-2,3-diol (pinacol), 2-methylbutane-2,3-diol, 1,2-diphenylethane-1,2-diol, 2-methylpentane-2,4-diol, 1,2-dihydroxybenzene (catechol), 2,2′-azanediyldiethanol, 2,2′-(butylazanediyl)diethanol, 2,3-dihydroxysuccinic acid (tartaric acid) and its esters, or (OR⁴)₂ originates in particular from diacids such as 2,2′-(methylazanediyl)diacetic acid (mida),
[symbol] R¹ and R² represent, independently of one another, linear or branched chains having from 1 to 30 carbon atoms, R¹ and R² being saturated or unsaturated, unsubstituted or substituted by a halogen atom,
   and in the case of an unsaturation, the C═C double bond optionally being substituted by a fluorine, chlorine, or bromine atom or by a —CF₃ group,
   and in the case when R¹ and R² only comprise a single carbon, they are selected from the groups of Formula “—CHV—”, in which V represents —H, —F, —Cl or —Br, Y₁ and Y₂ then being equal to the phosphonate group —P(O)(OR⁴)₂, R⁴ having the meaning given above.
      The compounds of general formula I represented above both contain chains designated X₁ and X₂. These chains all comprise an amide function —NH—CO—.
      X₁ and X₂ possess as terminal portion Y₁ and Y₂, which can be hydrogen if this chain is not functionalized in the terminal position. If the terminal carbon is functionalized, Y₁ and Y₂ can be carboxylic acid functions or the ester form thereof. Y₁ and Y₂ can also be alcohol functions, protected or not. In the case of a protected alcohol, Y₁ and Y₂ can be derivatives of glycoside compounds, i.e. of sugars, with the bond between the terminal —OH and the anomeric oxygen. These sugars are the α- or β-furanoses and the α- or β-pyranoses.
      An alcohol can also be protected by conversion to an ether function using, for example, the tetrahydropyran derivative represented above, optionally in its open form.
      Y₁ and Y₂ can also be primary, secondary or tertiary amine functions, optionally protected in the form of amide —NHCORₑ, or of carbamate —NHCOOR_{f},
      Y₁ and Y₂ can also be phosphonate groups —P(O)(OR⁴)₂, R⁴ having the meaning given above.
      In the formulae representing the chains designated X₁ and X₂, the R¹ and R² radicals represent the carbon chain comprised between the amide function —NH—CO— attached to the ring by nitrogen, and the terminal group designated Y₁ or Y₂. The R¹ and R² radicals then comprise from 1 to 30 carbon atoms. R¹ and R² can be saturated or unsaturated, in this case comprising one, two or three carbon/carbon double bonds. One or more of the carbons of one or more C═C bond(s) can carry a fluorine, chlorine, or bromine atom or a —CF₃ group.
      The indices m and n make it possible to vary the size of the ring. “m” can take the values 1, 2 or 3. “n” can take the values 0 or 1. The compounds forming the subject of the invention are derivatives of cyclopropanes, of cyclobutanes or of cyclopentanes functionalized by two amide chains, each bound to a carbon atom of the ring.
      When n=0 and m=1, these compounds are derivatives of cyclopropane.
      When the sum n+m=2, these compounds are derivatives of cyclobutane.
      When the sum n+m=3, these compounds are derivatives of cyclopentane.
      The compounds n+m≧4 are not covered by the present invention.
      As is defined above, the R¹ and R² portions can be identical or different. The same applies to the terminal portions Y₁ and Y₂. As a result, the novel compounds belong to two families. We shall in fact draw a distinction between:
the compounds in which the amide side chains are both attached to the ring by the nitrogen atom, both having the same R¹ and Y₁ portions. These compounds are symmetrical.
the compounds in which the amide side chains are both attached to the ring by the nitrogen atom, but differ by the respective natures of R¹ and R² and/or of Y₁ and Y₂. These compounds are described as asymmetrical.
   Moreover, the compounds represented by general formula I display cis or trans stereochemistry. They are “cis” if the two chains are located on the same side of the ring and “trans” in the opposite case.

A subject of the invention is the compounds in which X₁ and X₂ are identical or different and correspond to Formula I_{A} or I_{B}

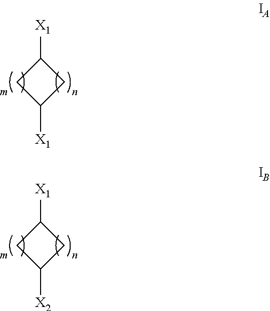

in which
X₁, X₂, m and n have the meanings given above.
The compounds I_{A} are symmetrical as the side chains are strictly identical.
The compounds I_{B} are asymmetrical as the side chains are different.
   For all the compounds I_{A} and I_{B}, it is possible to have the cis or trans configurations.

A subject of the invention is the compounds of Formula II

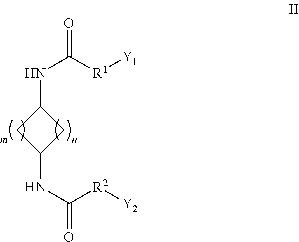

in which
R¹, R², Y₁, Y₂, m and n have the meanings given above,
R¹, R² are identical or different,
Y₁ and Y₂ are identical or different.
The compounds II have branchings on the ring at the nitrogen atom, for the 2 side chains. The symmetrical and asymmetrical compounds are included here.
For all the compounds II, it is possible to have the cis or trans configurations.

Advantageously, the compounds of the invention have Formula II_{cis}

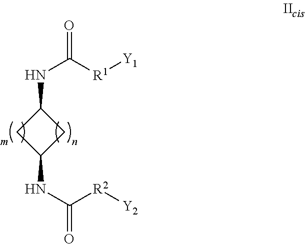

in which
R¹, R², Y₁, Y₂, m and n have the meanings given above,
R¹, R² are identical or different,
Y₁ and Y₂ are identical or different,
m=1, 2, 3,
n=0, 1,
provided that m+n is different from 4.
The compounds II_{cis} are therefore exclusively of cis stereochemistry. They are constituted by a carbon ring with 3, 4 or 5 atoms, to which the 2 amide chains are attached.

Advantageously, the compounds of the invention have Formula II_{A cis}

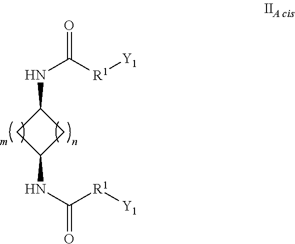

in which
R¹, Y₁, m and n have the meanings given above,
m=1, 2, 3,
n=0, 1,
provided that m+n is different from 4.
The compounds II_{A cis} are therefore exclusively of cis stereochemistry. They are constituted by a carbon ring with 3, 4 or 5 atoms, to which the 2 amide chains are attached, these two chains being strictly identical; these compounds are symmetrical.

Advantageously, the compounds of the invention have Formula II_{B cis}

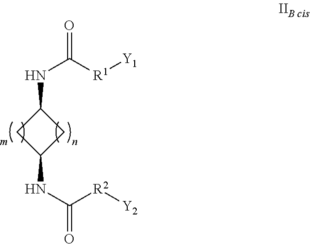

in which
R¹, R², Y₁, Y₂, m and n have the meanings given above,
provided that if R¹ and R² are identical, then Y₁ and Y₂ are different,
provided that if R¹ and R² are different, then Y₁ and Y₂ are identical or different,
m=1, 2, 3,
n=0, 1,
provided that m+n is different from 4.
The compounds II_{B cis} are therefore exclusively of cis stereochemistry. They are constituted by a carbon ring with 3, 4 or 5 atoms, to which the 2 amide chains are attached, these two chains being different; these compounds are asymmetrical.

Also advantageously, the compounds of the invention have Formula IIₜᵣₐₙₛ

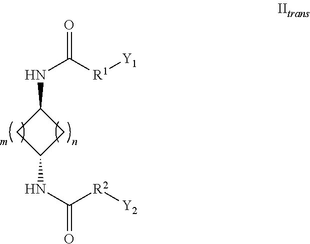

in which
R¹, R², Y₁, Y₂, m and n have the meanings given above,
R¹, R² are identical or different,
Y₁ and Y₂ are identical or different,
m=1, 2, 3,
n=0, 1,
provided that m+n is different from 4.
The compounds IIₜᵣₐₙₛ are therefore exclusively of trans stereochemistry. They are constituted by a carbon ring with 3, 4 or 5 atoms, to which the 2 amide chains are attached.

Advantageously, the compounds of the invention have Formula II_{A trans}

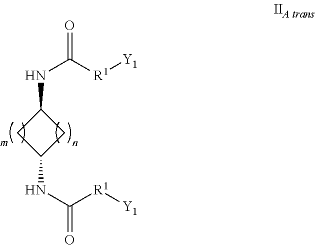

in which
R¹, Y₁, m and n have the meanings given above,
m=1, 2, 3,
n=0, 1,
provided that m+n is different from 4.
The compounds II_{A trans} are therefore exclusively of trans stereochemistry. They are constituted by a carbon ring with 3, 4 or 5 atoms, to which the 2 amide chains are attached, these two chains being strictly identical; these compounds are symmetrical.

Advantageously, the compounds of the invention have Formula II_{B trans}

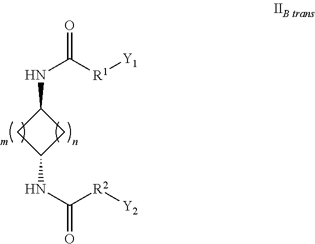

in which
R¹, R², Y₁, Y₂, m and n have the meanings given above,
provided that if R¹ and R² are identical, then Y₁ and Y₂ are different,
provided that if R¹ and R² are different, then Y₁ and Y₂ are identical or different,
m=1, 2, 3,
n=0, 1,
provided that m+n is different from 4.
The compounds II_{B trans} are therefore exclusively of trans stereochemistry. They are constituted by a carbon ring with 3, 4 or 5 atoms, to which the 2 amide chains are attached, these two chains being different; these compounds are asymmetrical.

A subject of the invention is the compounds of Formula I in which n is equal to 0 and m is equal to 1, and corresponding to Formulae V_{A} or V_{B}

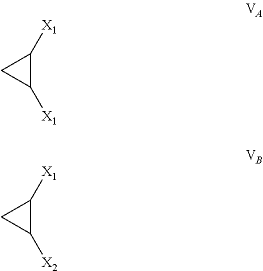

in which
X₁, X₂, m and n have the meanings given above.
In this case, m=1 and n=0. The compounds V_{A} and V_{B} are therefore derivatives of a ring with three carbon atoms, rings optionally formed from an olefinic starting compound. They are therefore derivatives of cyclopropane. The two amide chains are each attached to a carbon atom of the ring.
The compounds V_{A} are symmetrical; the compounds V_{B} are asymmetrical.
For all the compounds V_{A} and V_{B}, it is possible to have the cis or trans configurations.

A subject of the invention is the compounds of Formula I in which n+m is equal to 2, and corresponding to general formula XXII

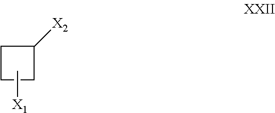

in which
X₁, X₂, n and m have the meanings given above.
In this case, the sum m+n=2. The compounds XXII are therefore derivatives of a ring with four carbon atoms. The compounds XXII are derivatives of cyclobutane. The two amide chains are each attached to a carbon atom of the ring. They can be carried by carbons that are adjacent or separated by another carbon atom of the ring.
The symmetrical and asymmetrical compounds are included.
For all the compounds XXII, it is possible to have the cis or trans configurations.

A subject of the invention is also the compounds of Formula I in which n is equal to 0 and m is equal to 2, and corresponding to Formulae XXII_{A} or XXII_{B}

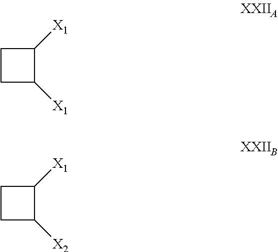

in which
X₁, X₂, m and n have the meanings given above.
In this case, the sum m+n=2 with n=0 and m=2. The compounds XXII_{A} and XXII_{B} are therefore derivatives of a ring with four carbon atoms. The compounds XXII are derivatives of cyclobutane. The two amide chains are each attached to a carbon atom of the ring; they are carried by adjacent carbon atoms.
The symmetrical and asymmetrical compounds are included.
For all the compounds XXII_{A}, XXII_{B}, it is possible to have the cis or trans configurations.

A subject of the invention is also the compounds of Formula I in which n is equal to 1 and m is equal to 1 and corresponding to Formulae XXII_{F} or XXII_{G}

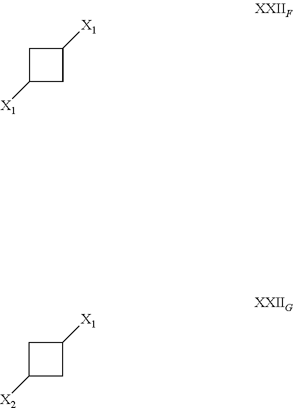

in which
X₁, X₂, m and n have the meanings designated above.
In this case, the sum m+n=2 with n=1 and m=1. The compounds XXII_{F} and XXII_{G} are therefore derivatives of a ring with four carbon atoms. The compounds XXII are derivatives of cyclobutane. The two amide chains are each attached to a carbon atom of the ring; they are carried by carbon atoms separated by another carbon atom, on the ring.
The symmetrical and asymmetrical compounds are included.
For all the compounds XXII_{F} and XXII_{G}, it is possible to have the cis or trans configurations.

A subject of the invention is also the compounds of Formula I in which n+m is equal to 3 and correspond to general formula VI

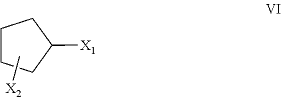

in which
X₁, X₂, n and m have the meanings given above.
In this case, the sum m+n=3. The compounds VI are therefore derivatives of a ring with five carbon atoms. The compounds VI are derivatives of cyclopentane. The two amide chains are each attached to a carbon atom of the ring. They can be carried by carbons that are adjacent or separated by another carbon atom of the ring.
The symmetrical and asymmetrical compounds are included.
For all the compounds VI, it is possible to have the cis or trans configurations.

The invention also relates to the compounds of Formula I in which n is equal to 0 and m is equal to 3 and corresponding to Formulae VI_{A} and VI_{B} shown below:

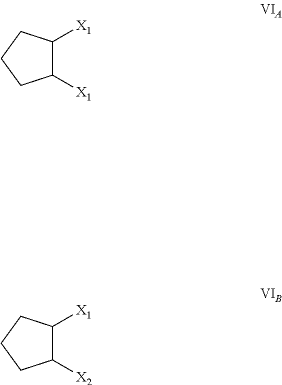

in which
X₁, X₂, m and n have the meanings given above.
In this case, the sum m+n=3 with n=0 and m=3. The compounds VI_{A} and VI_{B} are therefore derivatives of a ring with five carbon atoms. The compounds VI are derivatives of cyclopentane. The two amide chains are each attached to a carbon atom of the ring; they are carried by adjacent carbon atoms.
The symmetrical and asymmetrical compounds are included.
For all the compounds VI_{A} and VI_{B}, it is possible to have the cis or trans configurations.

A subject of the invention is also the compounds of Formula I in which n is equal to 1 and m is equal to 2 and corresponding to Formulae VI_{F} and VI_{G} shown below

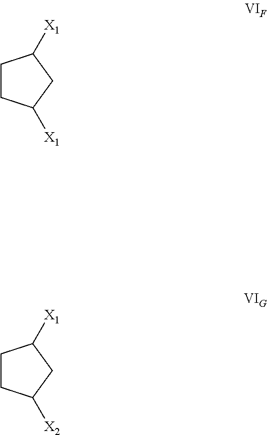

in which
X₁, X₂, m and n have the meanings given above.
In this case, the sum m+n=3 with n=1 and m=2. The compounds VI_{F} and VI_{G} are therefore derivatives of a ring with five carbon atoms. The compounds VI are derivatives of cyclopentane. The two amide chains are each attached to a carbon atom of the ring; they are carried by carbon atoms separated by another carbon atom, on the ring.
The symmetrical and asymmetrical compounds are included.
For all the compounds VI_{F} and VI_{G}, it is possible to have the cis or trans configurations.

Advantageously, the compounds of the invention have Formula VI_{F cis}

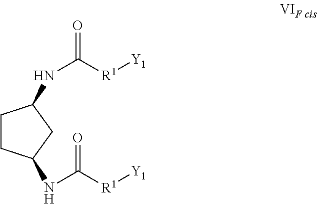

in which
R¹ and Y₁ have the meanings designated above.
The compounds VI_{F cis} are therefore exclusively of cis stereochemistry. They are constituted by a carbon ring with 5 atoms, the 2 amide chains being attached to the ring by carbons that are not adjacent. These two chains are identical; these compounds are symmetrical.

Advantageously, the compounds of the invention have Formula VI_{G cis}

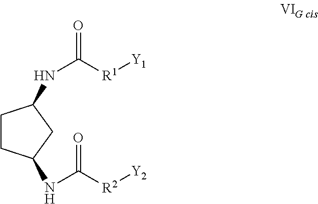

in which
R¹, R², Y₁, Y₂ have the meanings given above,
provided that if R¹ and R² are identical, then Y₁ and Y₂ are different,
provided that if R¹ and R² are different, then Y₁ and Y₂ are identical or different,
m=1, 2, 3,
n=0, 1,
provided that m+n is different from 4.
The compounds VI_{G cis} are therefore exclusively of cis stereochemistry. They are constituted by a carbon ring with 5 atoms, the 2 amide chains being attached to the ring by carbons that are not adjacent. These two chains are different; these compounds are asymmetrical.

Advantageously, the compounds of the invention have Formula VI_{F trans}

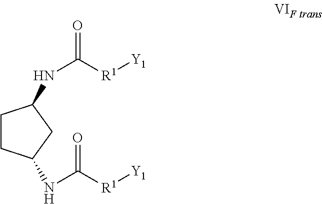

in which
R¹ and Y₁ have the meanings designated above.
The compounds VI_{F trans} are therefore exclusively of trans stereochemistry. They are constituted by a carbon ring with 5 atoms, the 2 amide chains being attached to the ring by carbons that are not adjacent. These two chains are identical; these compounds are symmetrical.

Advantageously, the compounds of the invention have Formula VI_{G trans}

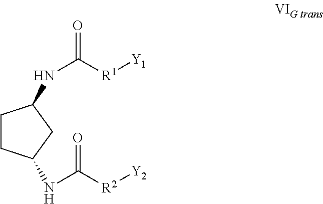

in which
R¹, R², Y₁, Y₂ have the meanings given above,
provided that if R¹ and R² are identical, then Y₁ and Y₂ are different,
provided that if R¹ and R² are different, then Y₁ and Y₂ are identical or different,
m=1, 2, 3,
n=0, 1,
provided that m+n is different from 4.
The compounds VI_{G trans} are therefore exclusively of trans stereochemistry. They are constituted by a carbon ring with 5 atoms, the 2 amide chains being attached to the ring by carbons that are not adjacent. These two chains are different; these compounds are asymmetrical.

A subject of the invention is the compounds in which the X₁ and X₂ groups are cis to one another, X₁ and X₂ having the meanings given above.

These compounds are “cis” isomers as the two chains carried by the ring are located on the same side of the ring.

A subject of the invention is the compounds in which the X₁ and X₂ groups are trans to one another, X₁ and X₂ having the meanings given above.

These compounds are “trans” isomers as the two chains carried by the ring are located on the same side of the ring.

A subject of the invention is the compounds represented by general formula I in which X₁ and X₂ are represented as below:

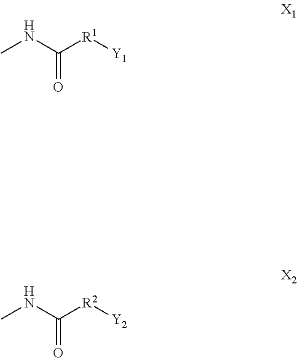

R¹ and R² representing, independently of one another, linear or branched chains, having from 1 to 30 carbon atoms,

the R¹—Y₁ and R²—Y₂ groups representing, independently of one another, one of the groups of the following formulae, the amine radical can optionally be substituted, the terminal hydroxyl radical can optionally be coupled to a glycoside residue selected from the α- or β-furanoses and the α- or β-pyranoses, or coupled to a linear aliphatic chain comprising one or more oxygen atoms, of formulae represented below,

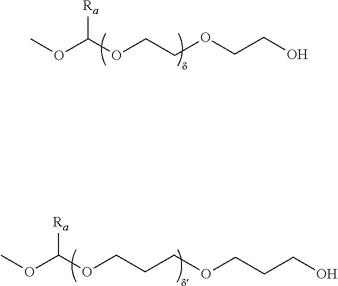

in which
δ varies from 1 to 12, δ′ varies from 1 to 5,
or a radical that can optionally be protected,
Rₐ representing a linear or branched alkyl group comprising from 1 to 4 carbon atoms, optionally substituted by one or more halogen atoms,

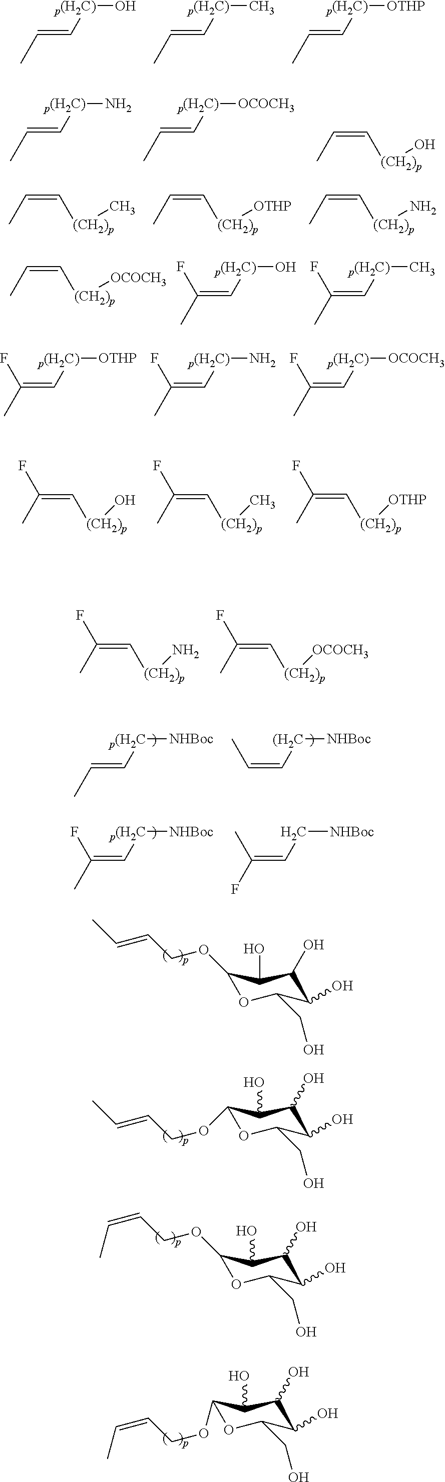

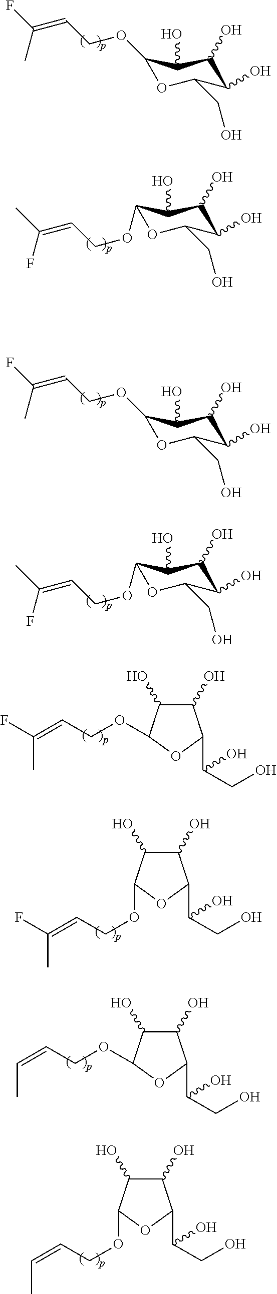

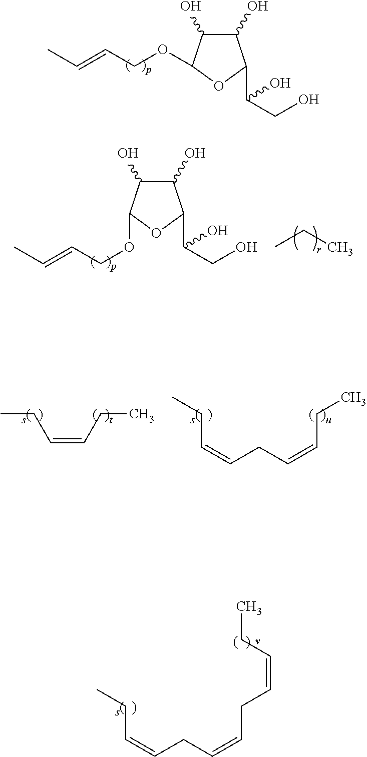

in which

p varies from 1 to 28,

r varies from 1 to 29,

s+t varies from 2 to 27,

s+u varies from 2 to 24,

s+v varies from 2 to 21.

The different natures of X₁ and X₂ have been represented as above.
The two side chains each comprise an amide function. The length of the chains varies, these two chains being selected independently of one another.
The R¹ and R² portions are saturated or unsaturated, then containing from one to three carbon/carbon double bonds optionally bearing a halogen atom or a —CF₃ group.
The terminal portions Y₁ and Y₂ are hydrogens, protected or unprotected alcohol functions, protected or unprotected amines, in particular in the form —NHBoc and derivatives thereof, carboxylic acids or esters, as has been described above.
The invention relates to the compounds corresponding to one of the following formulae. Compounds according to claim 1, of general formula I, shown below:

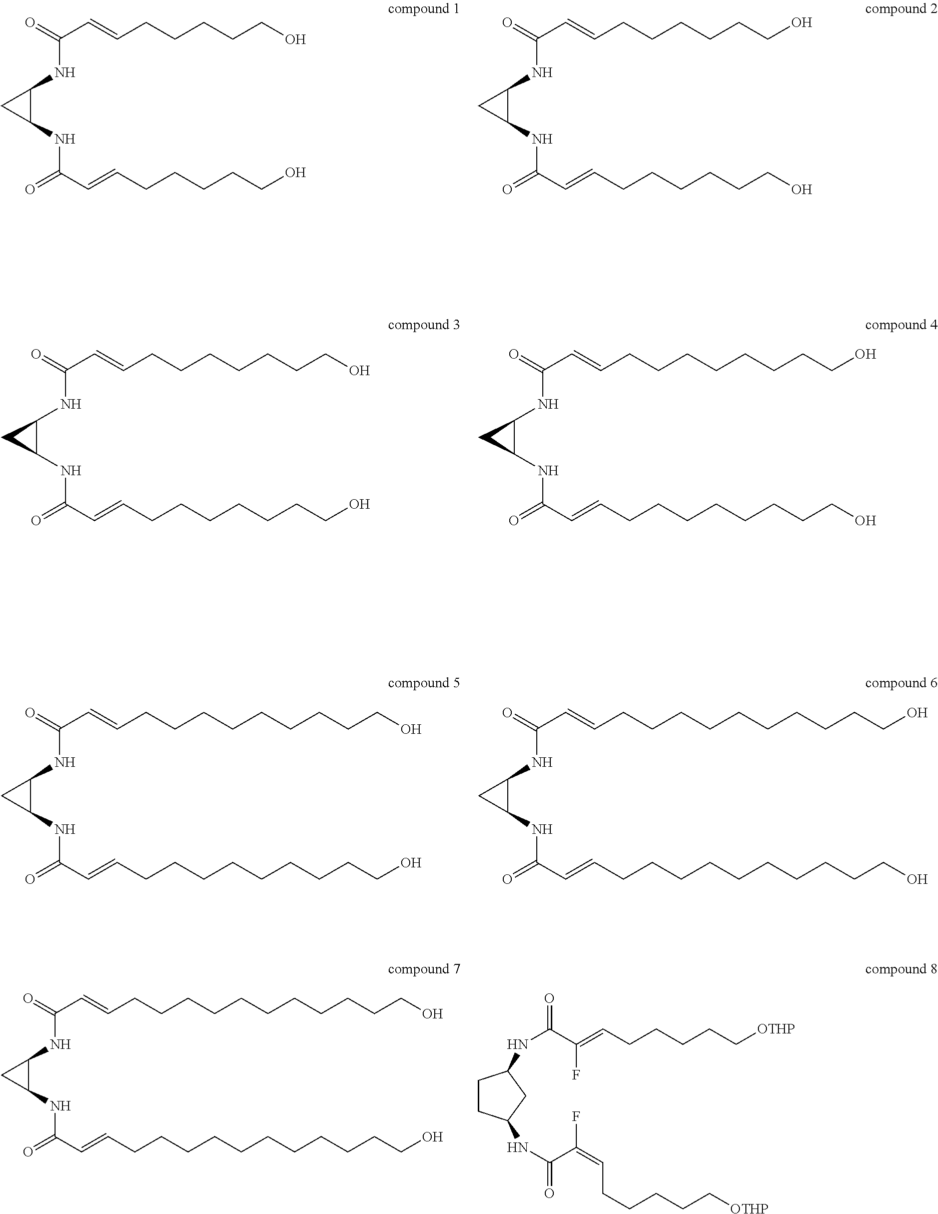

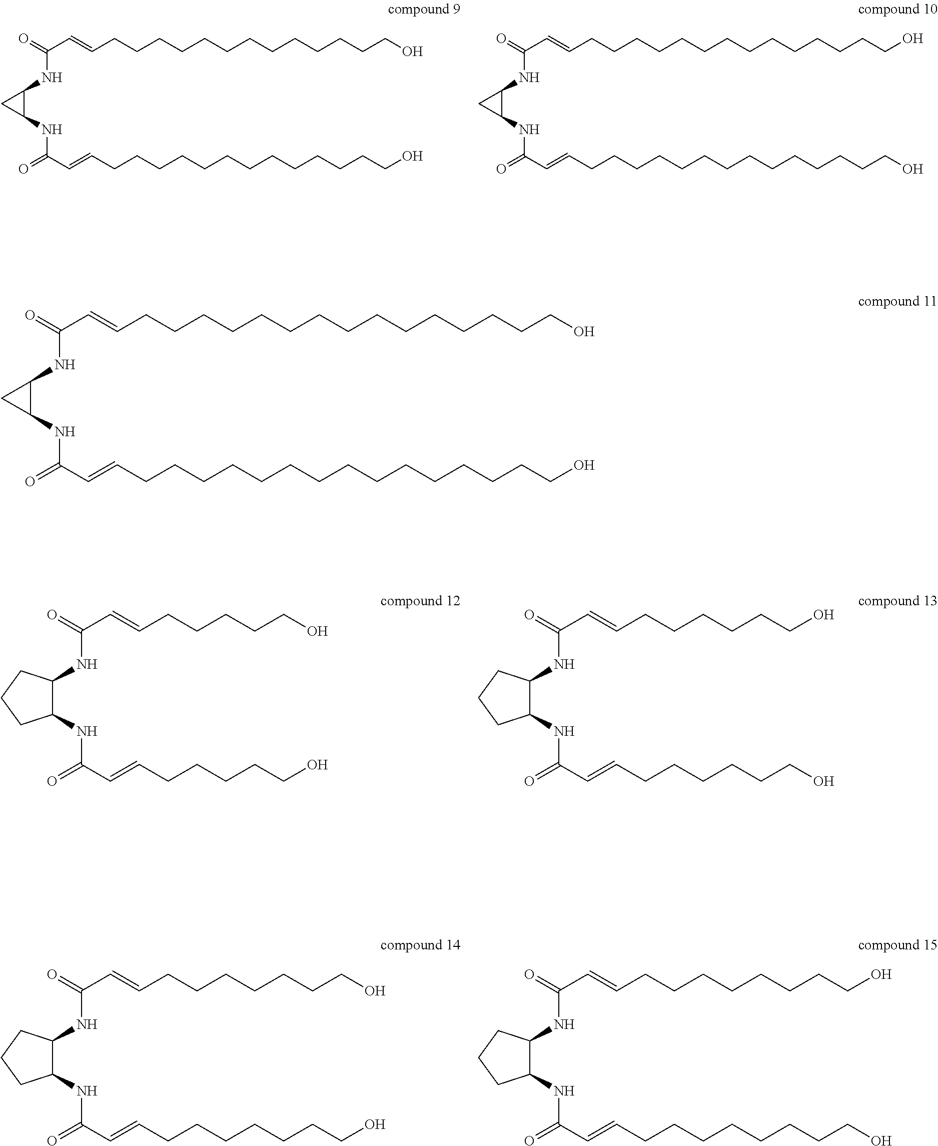

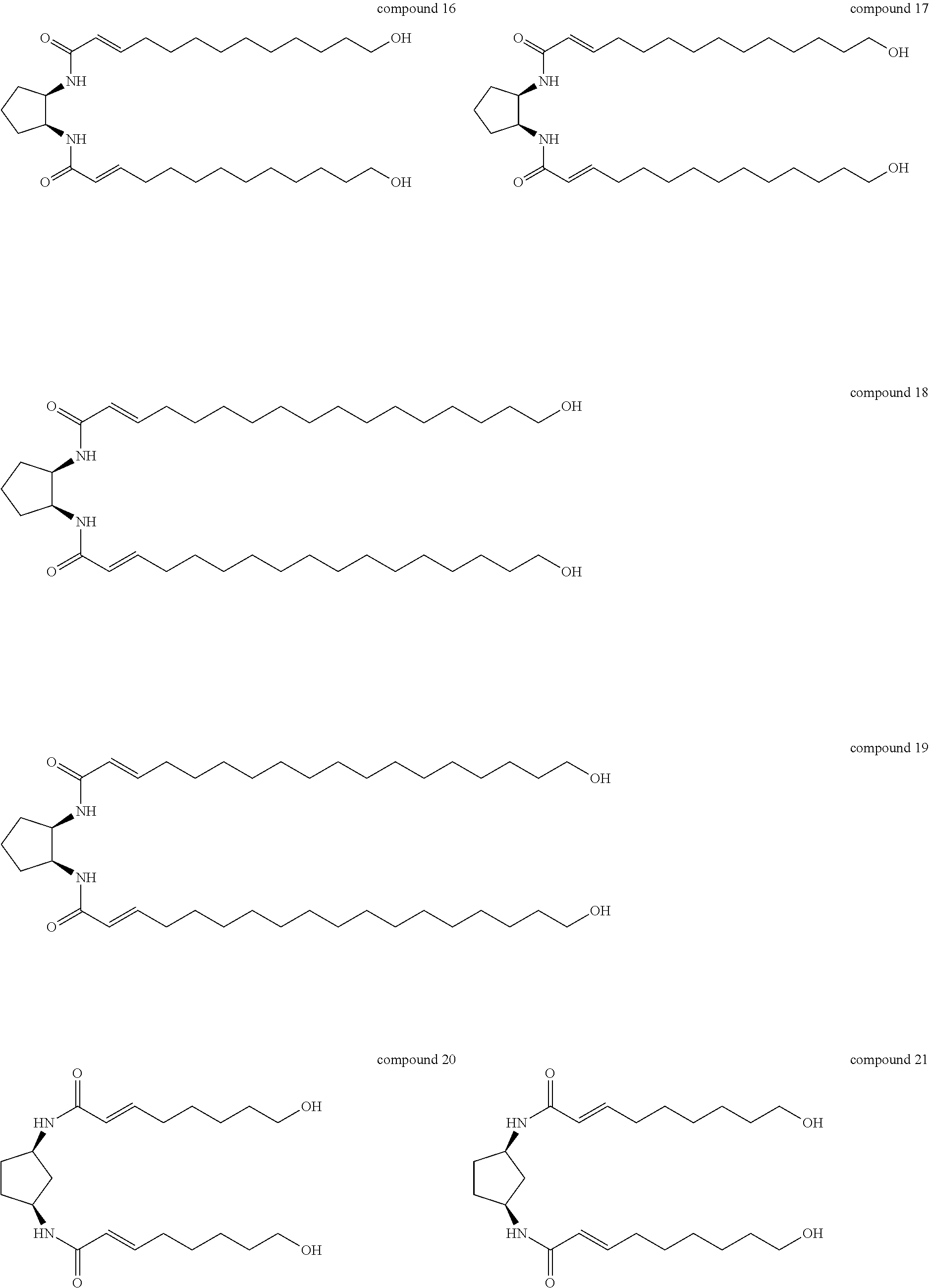

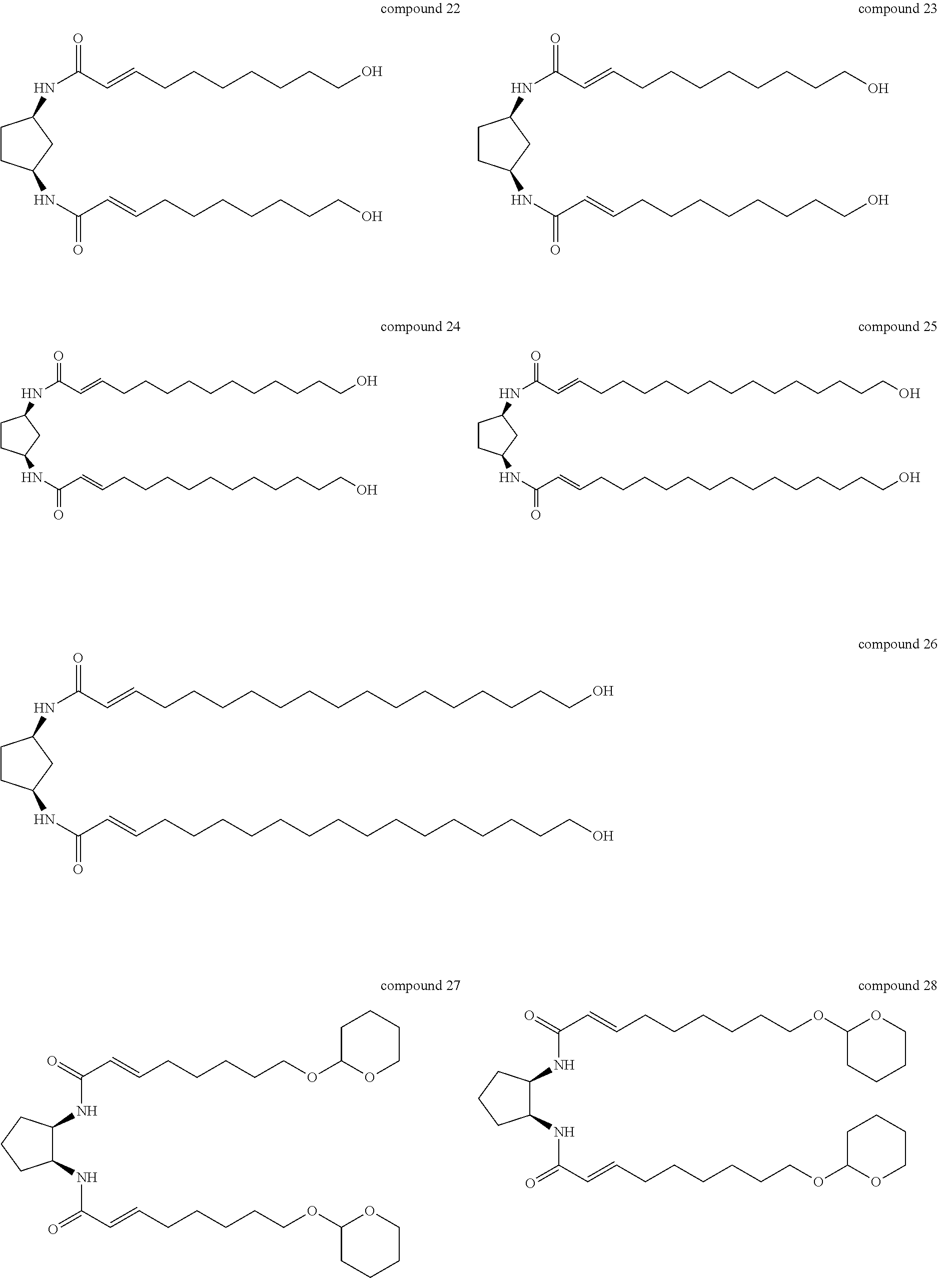

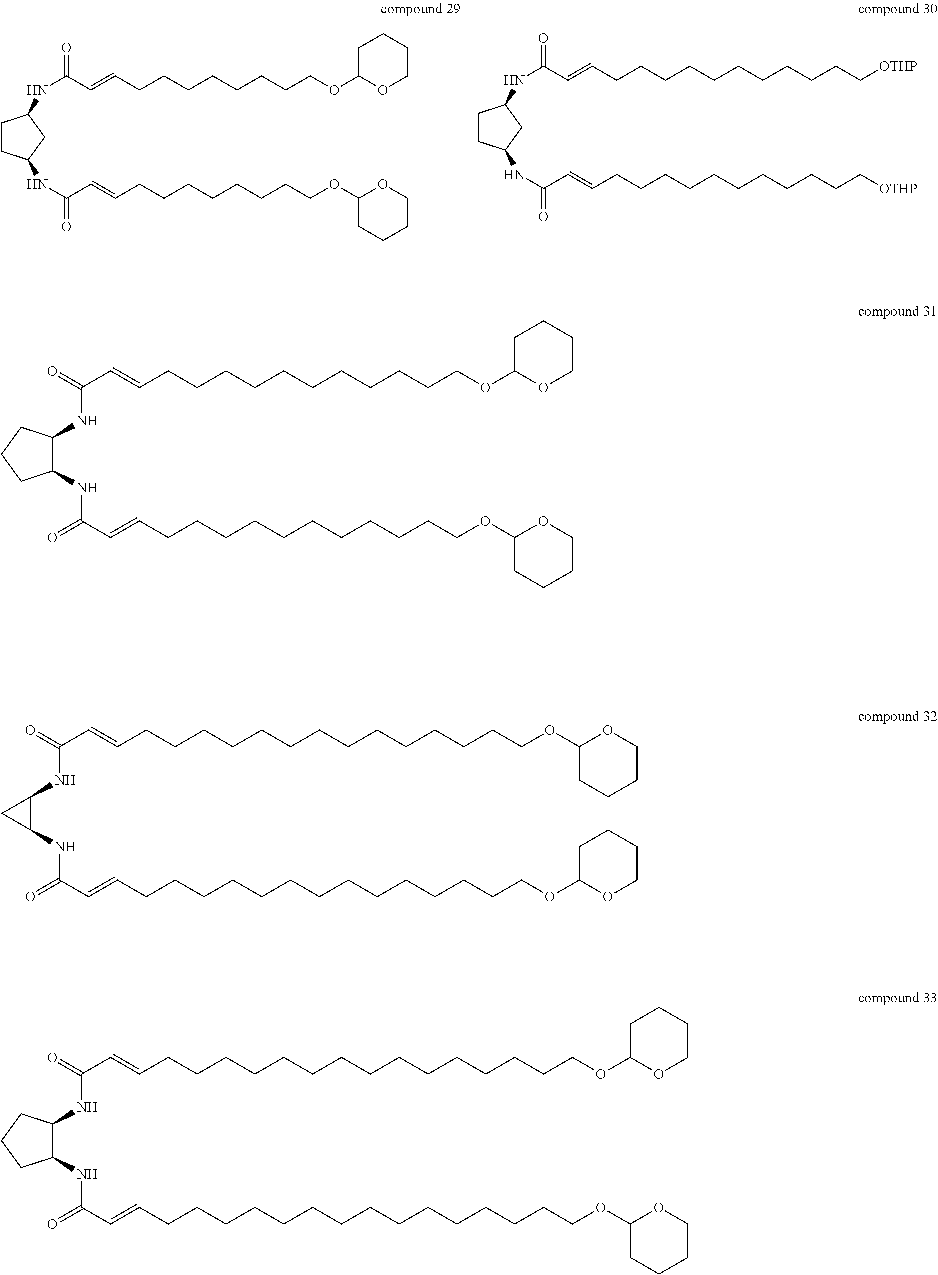

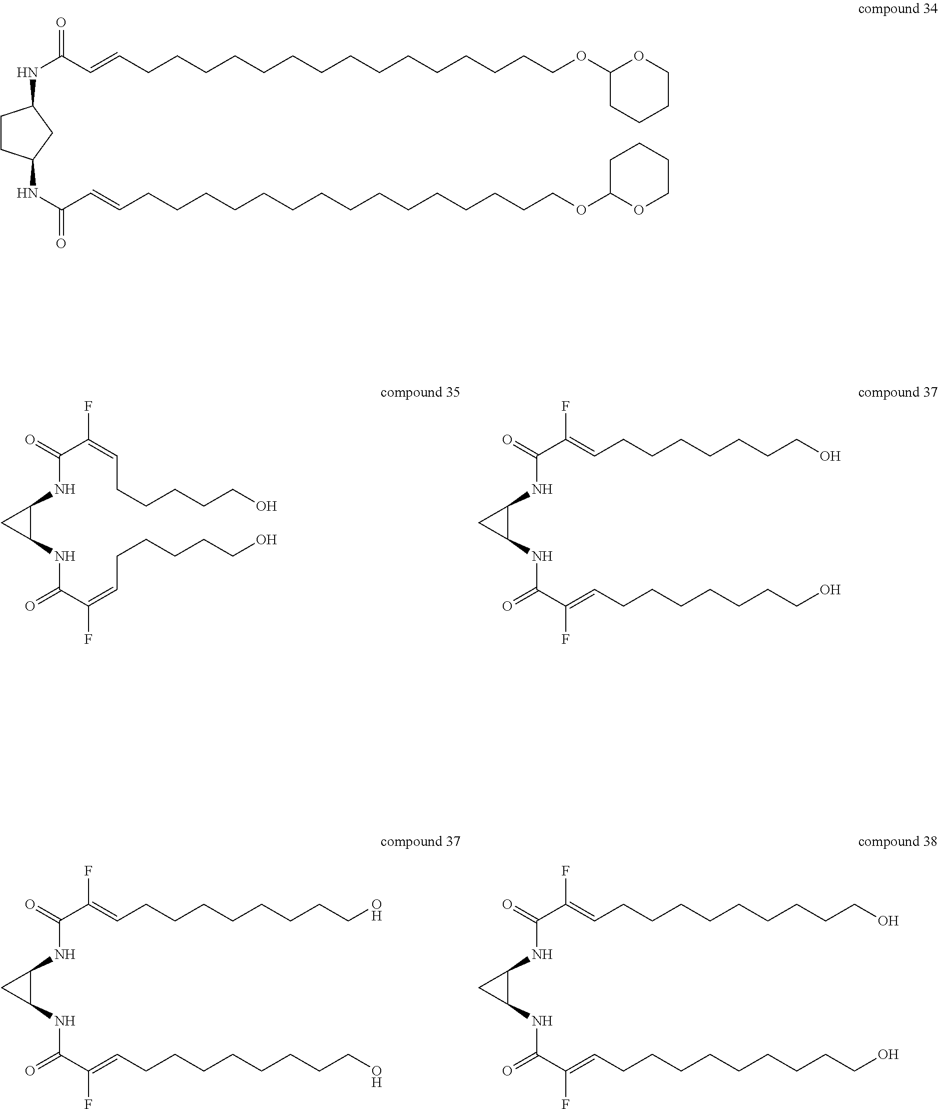

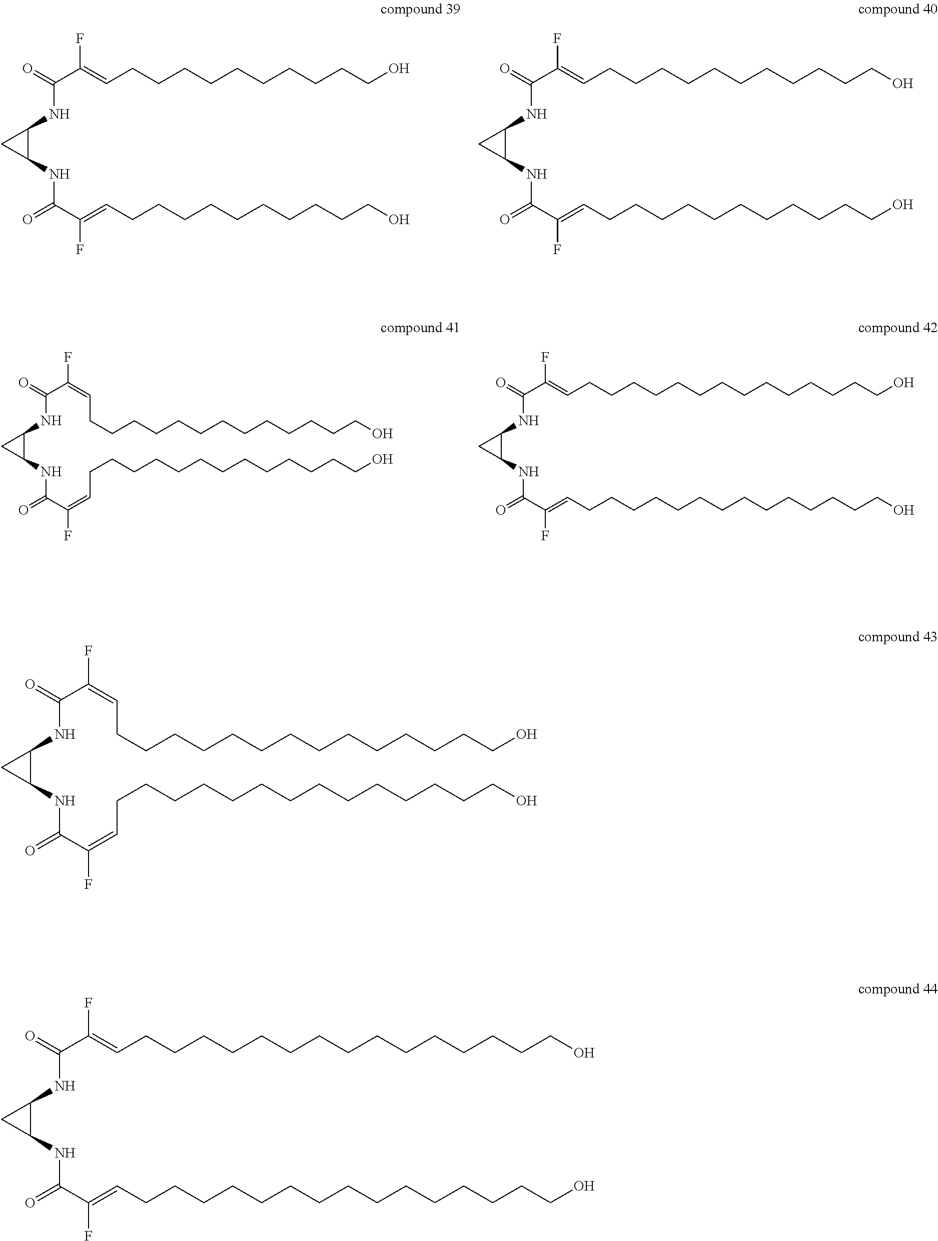

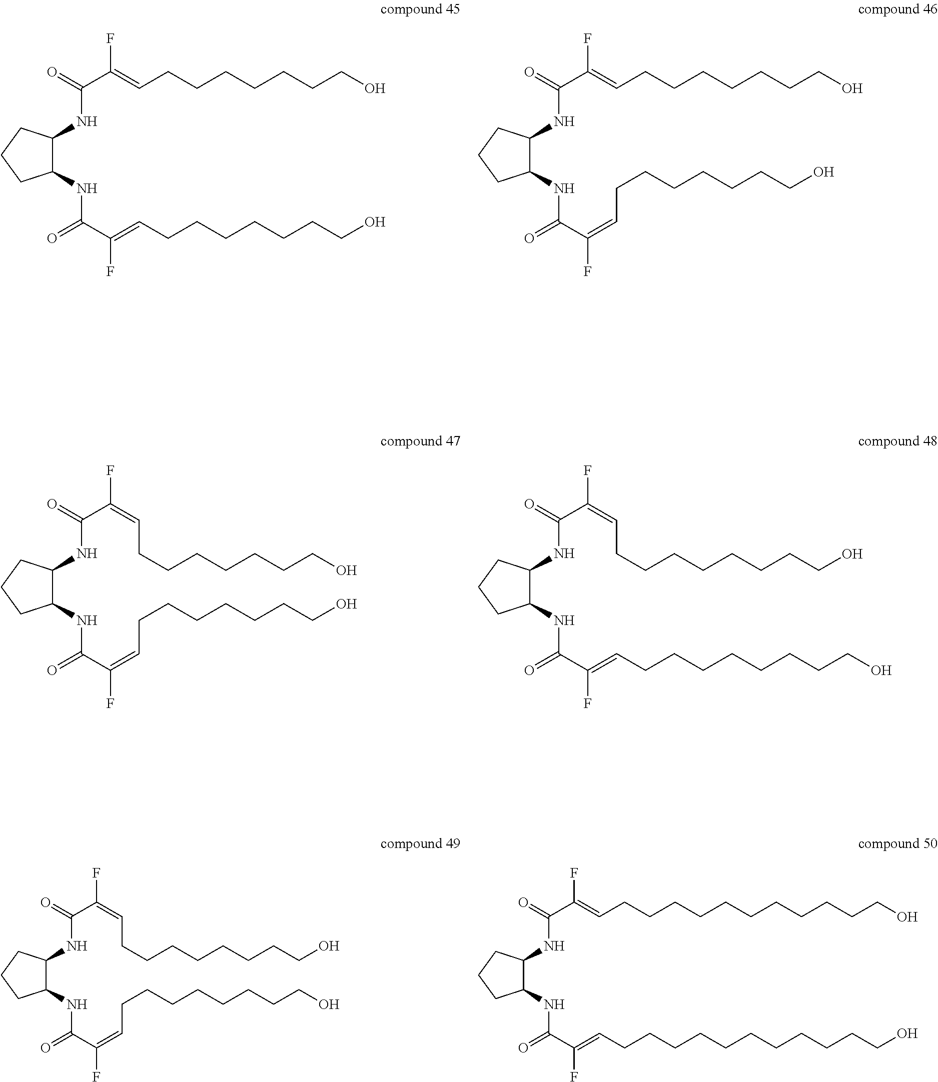

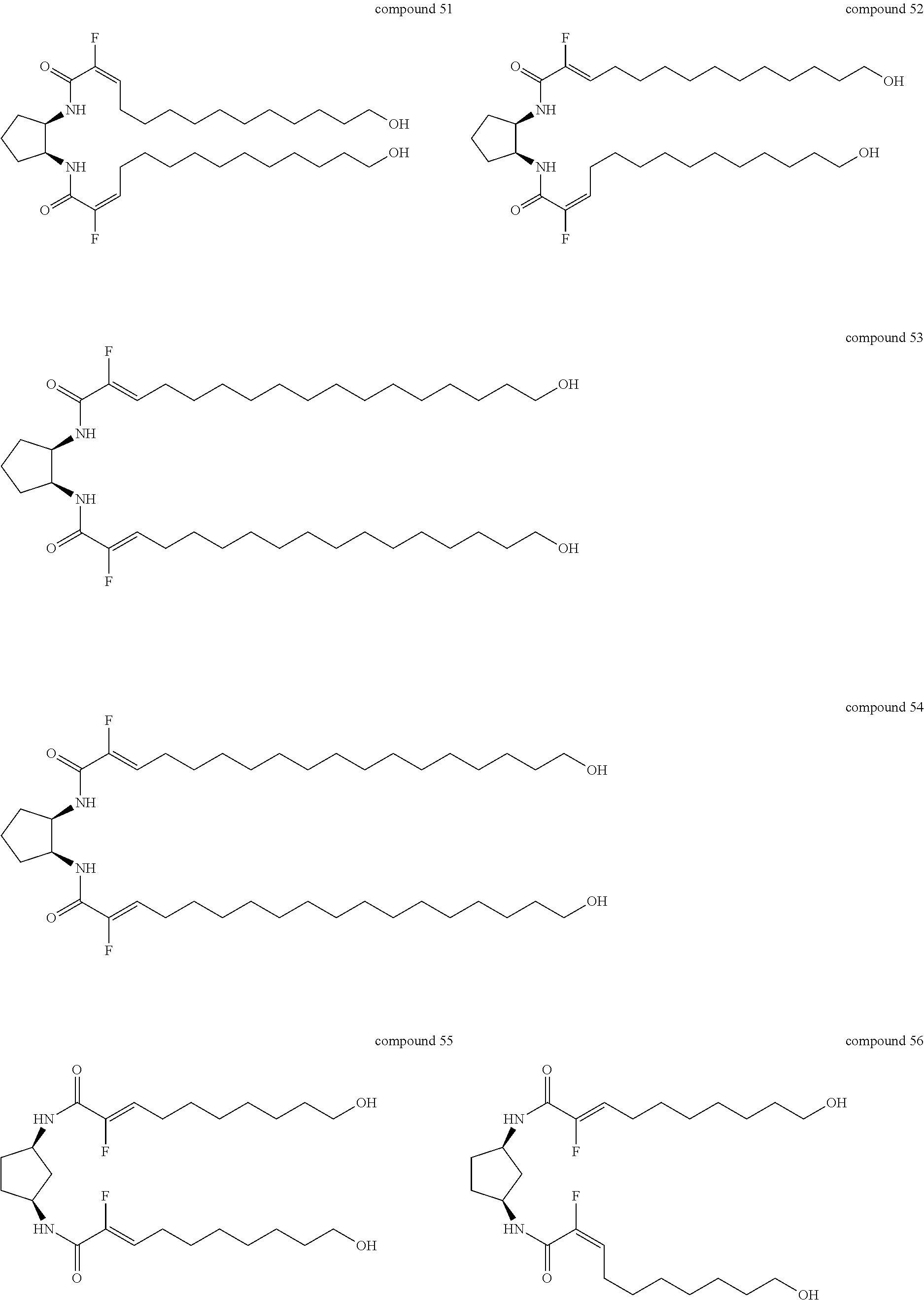

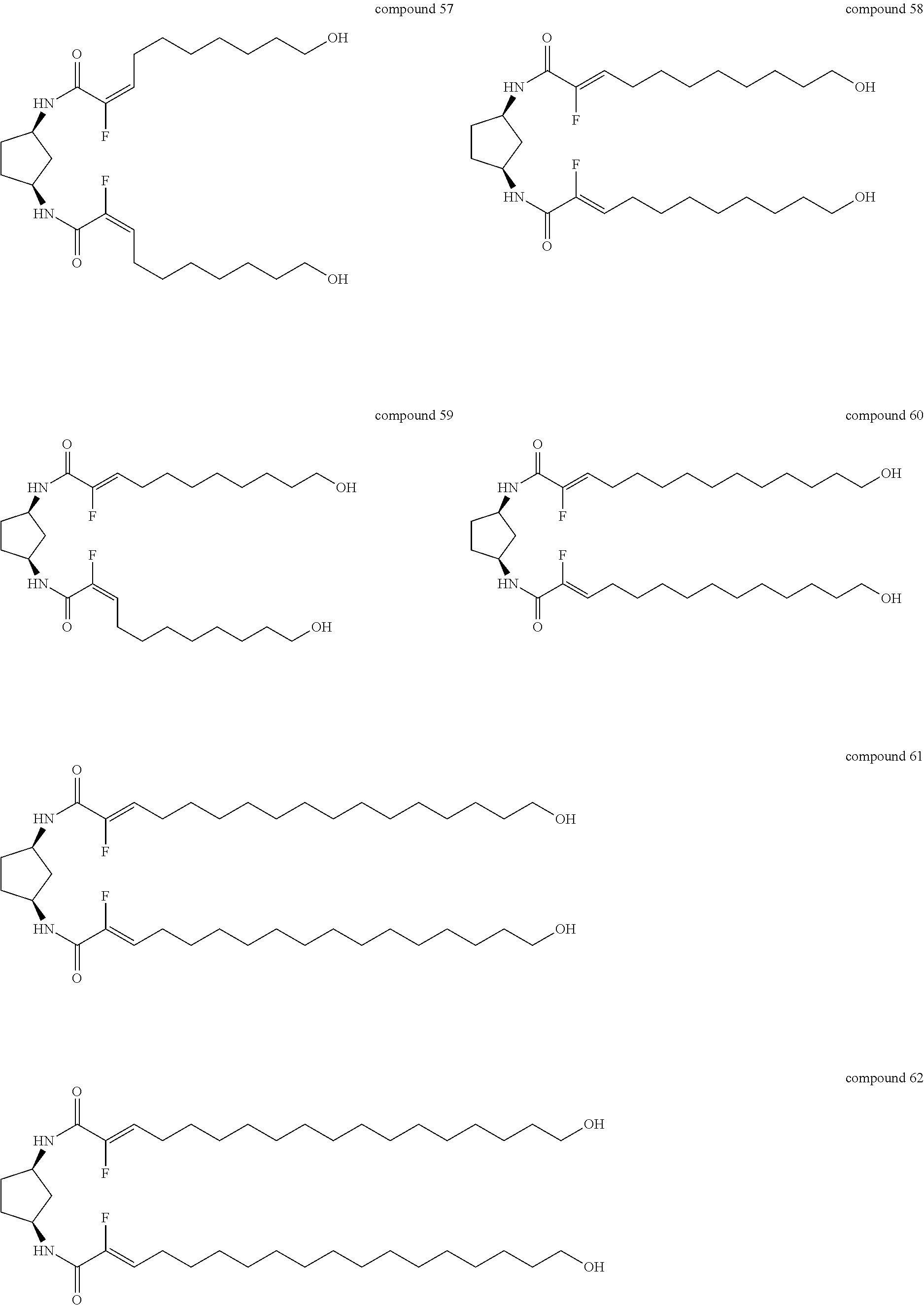

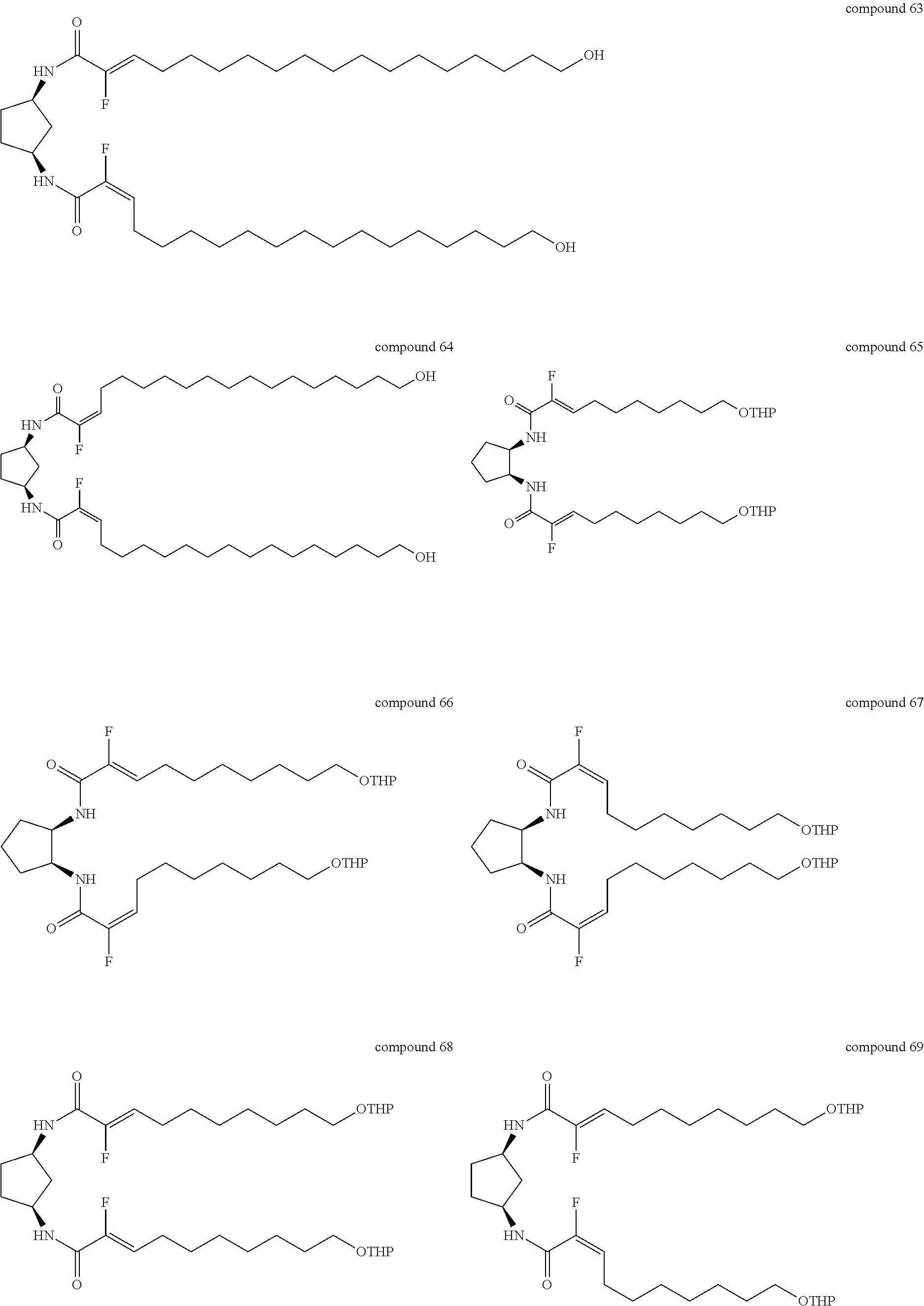

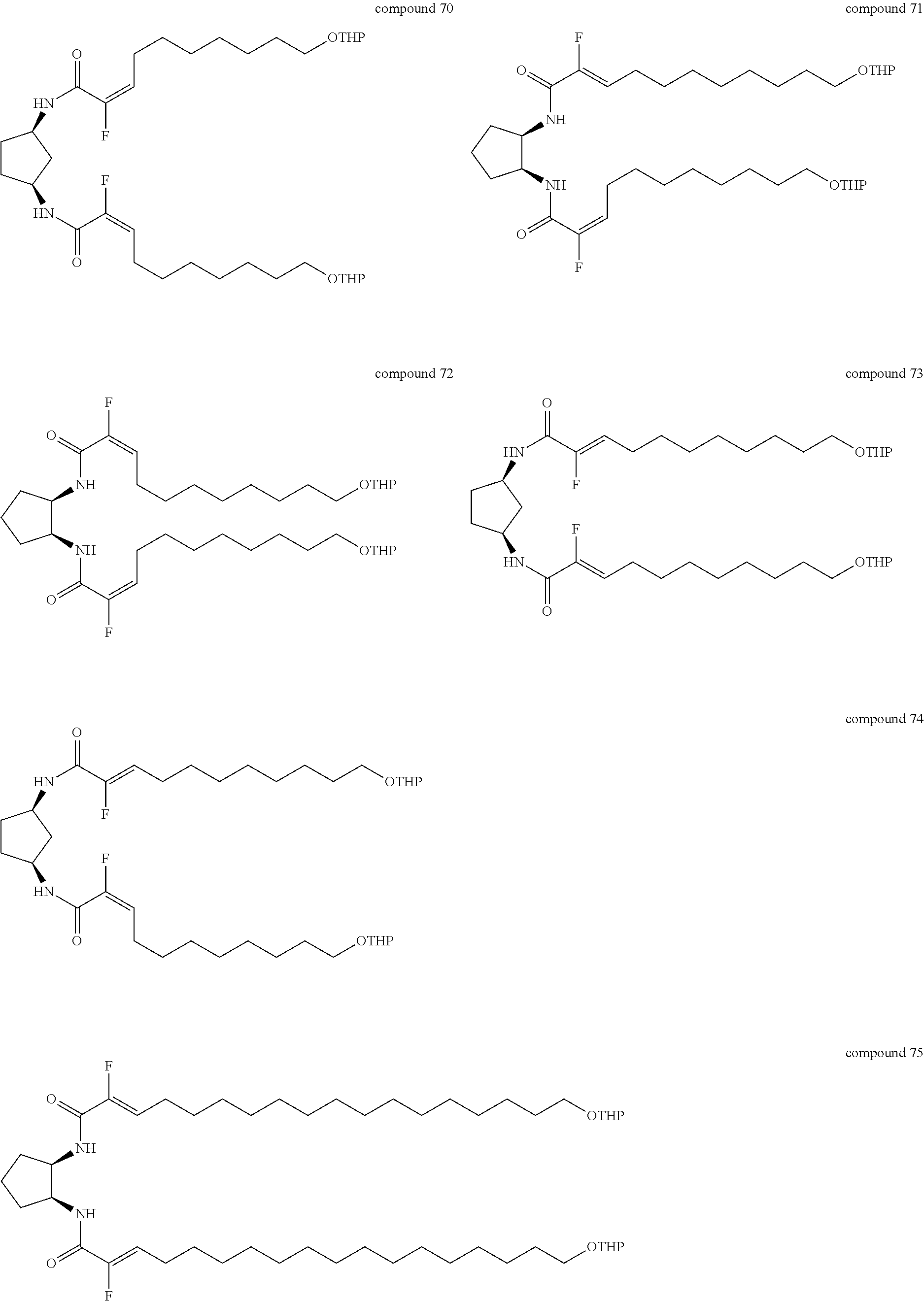

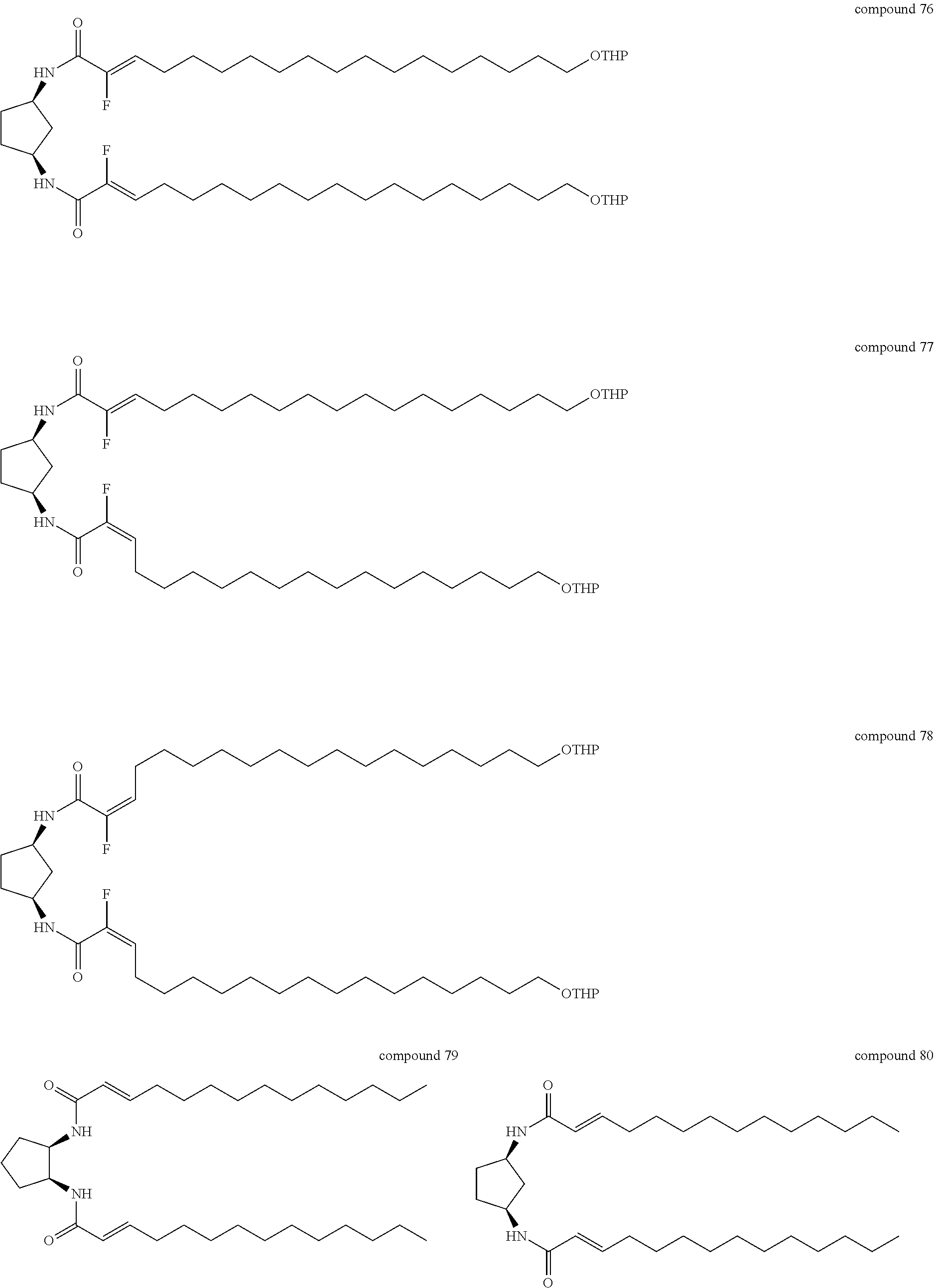

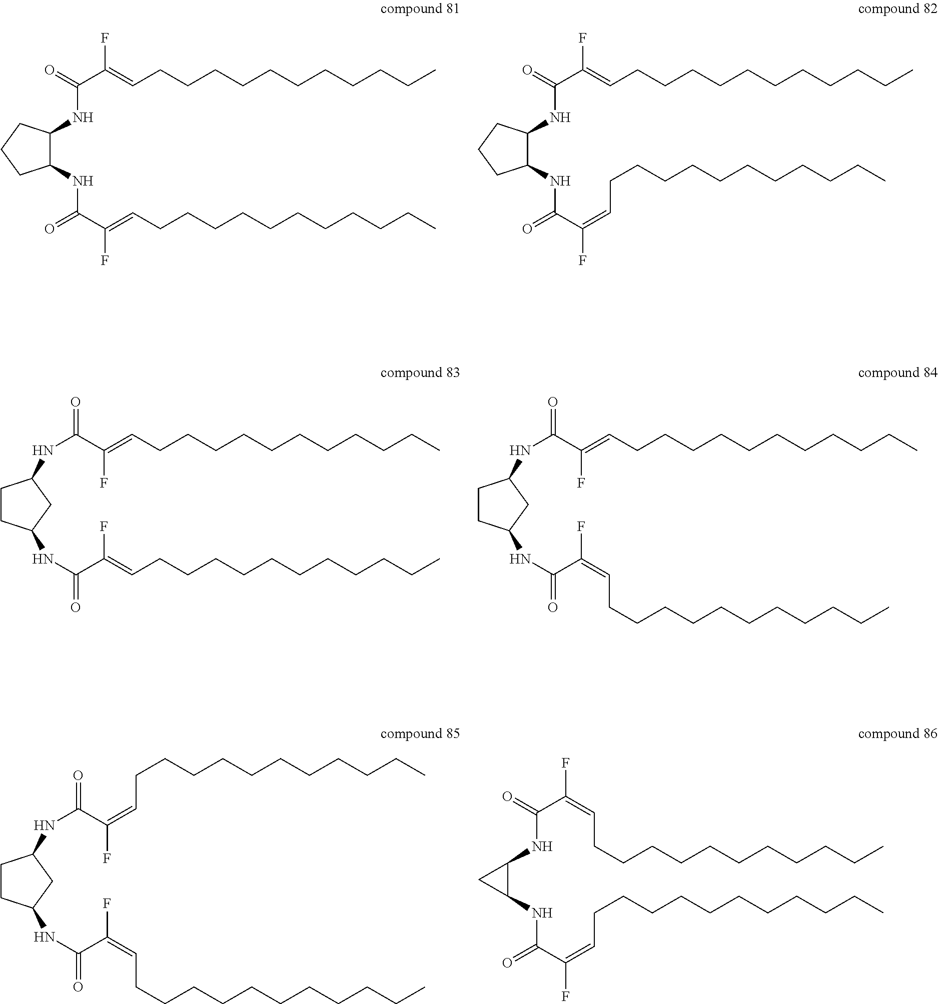

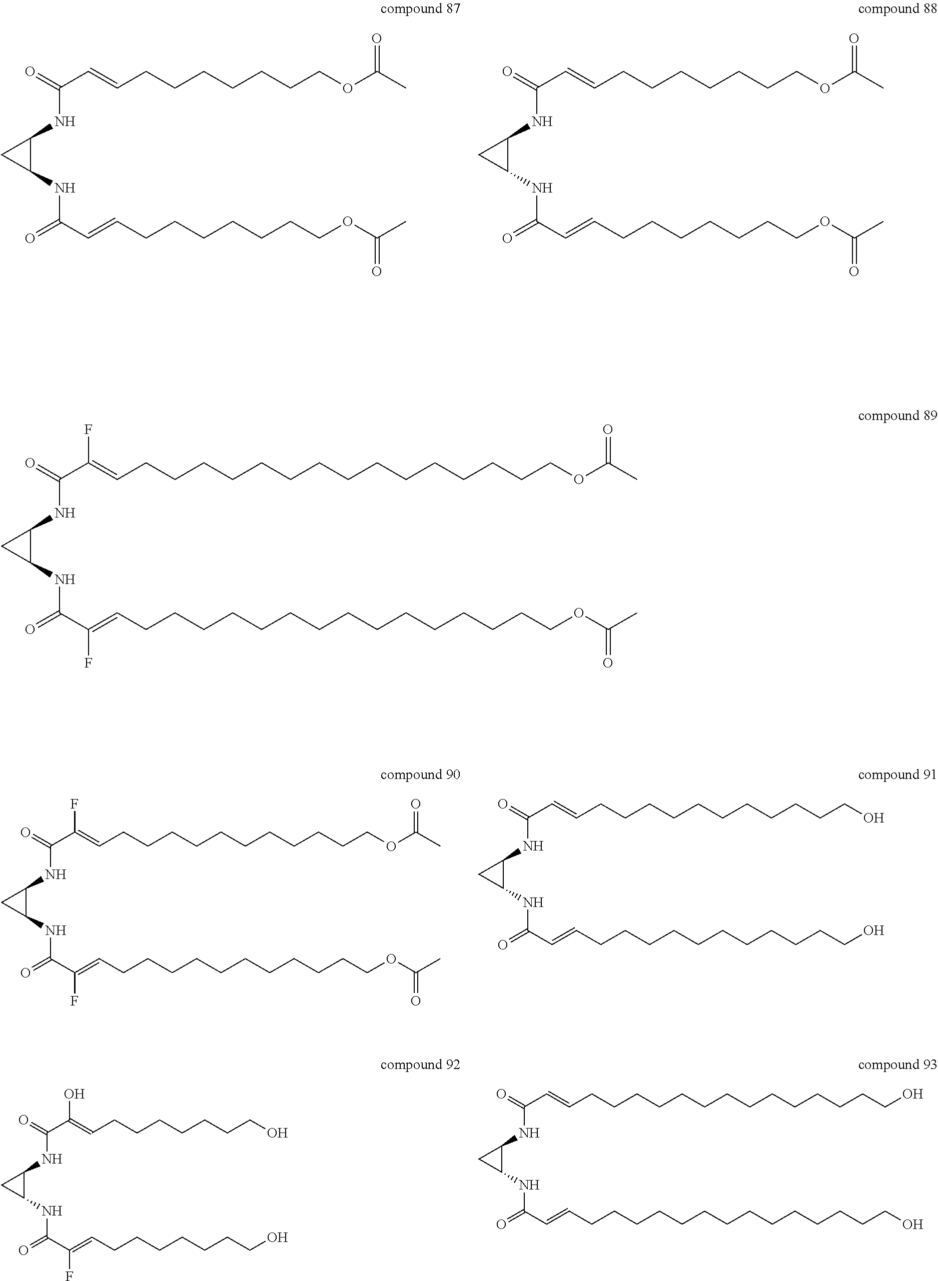

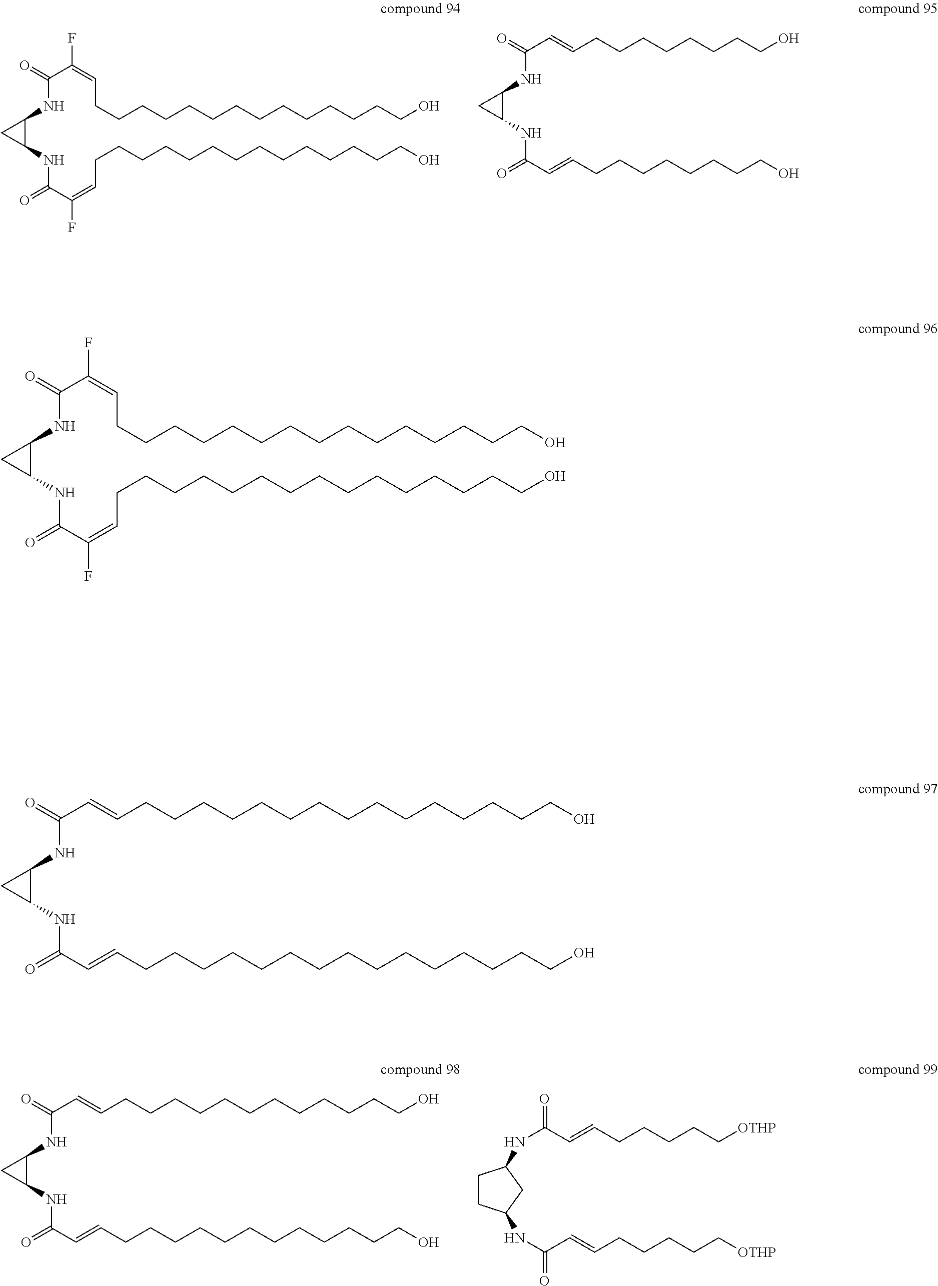

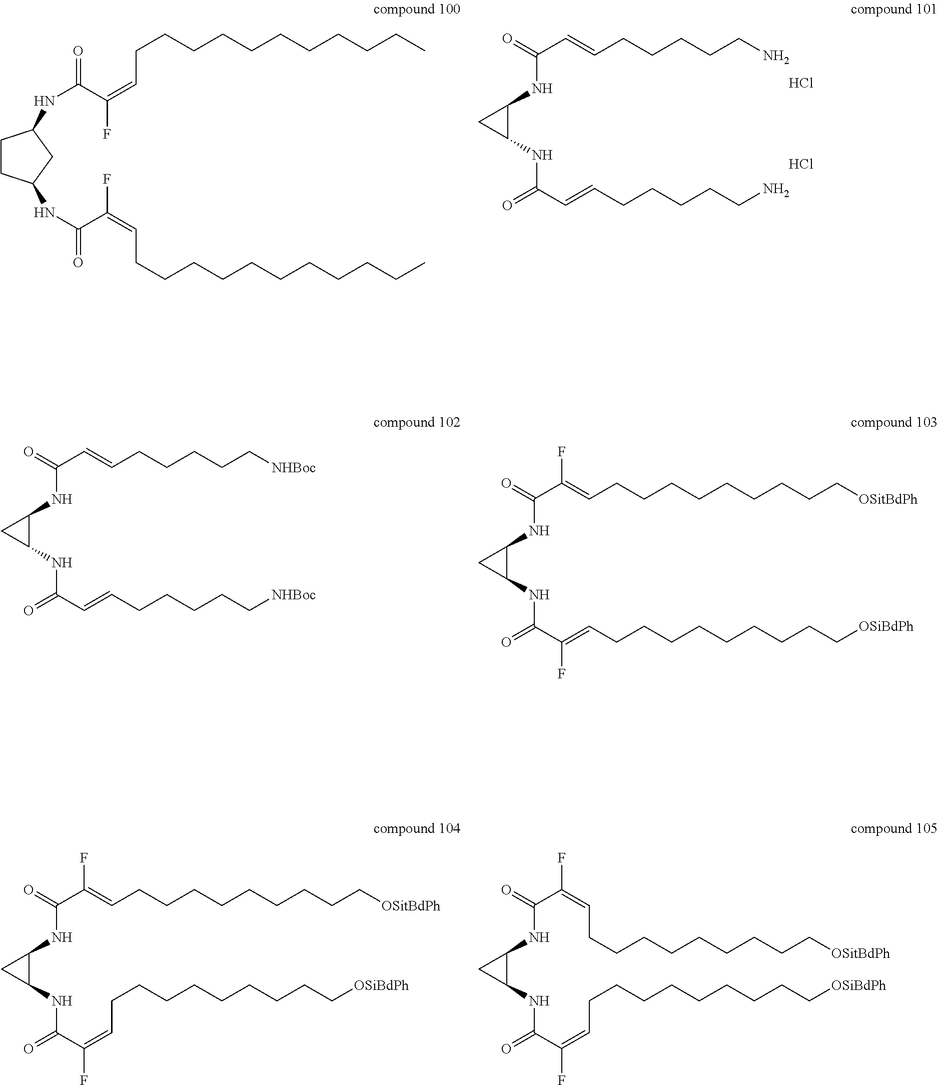

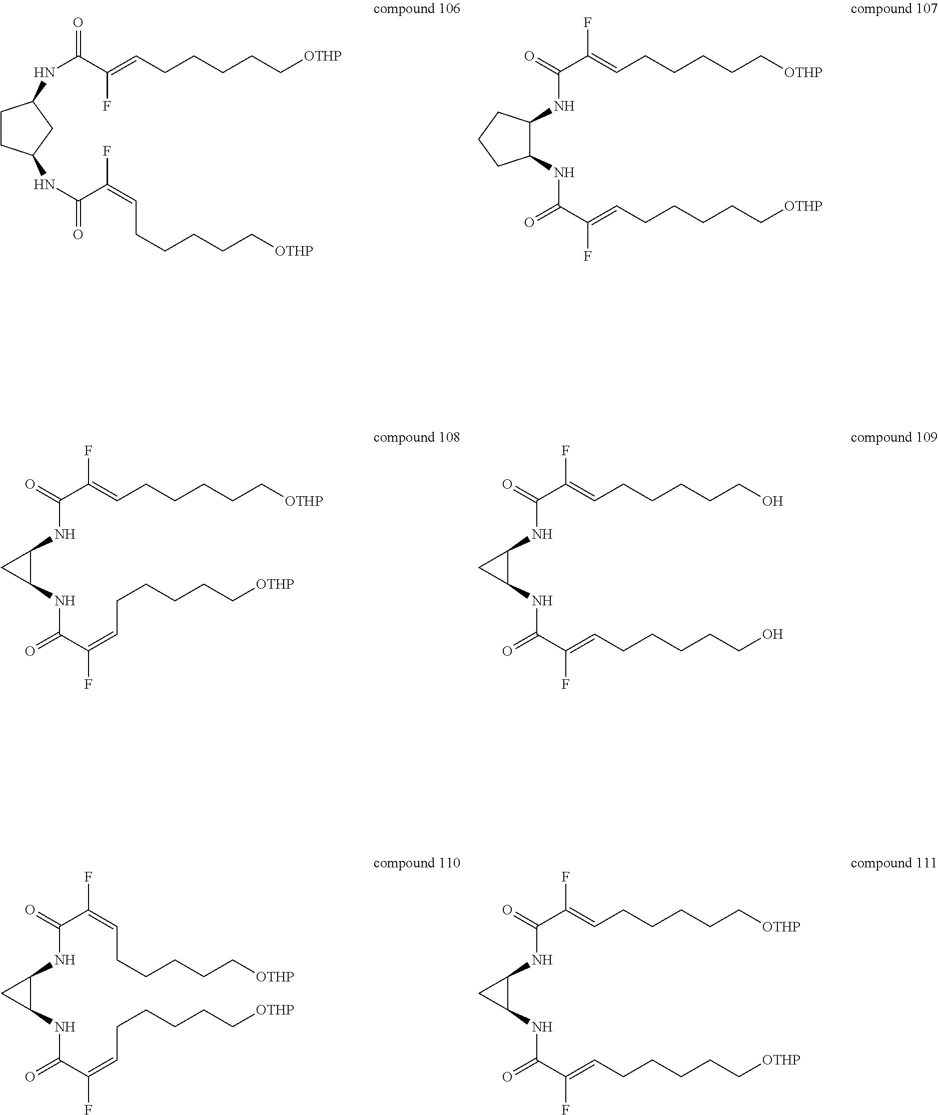

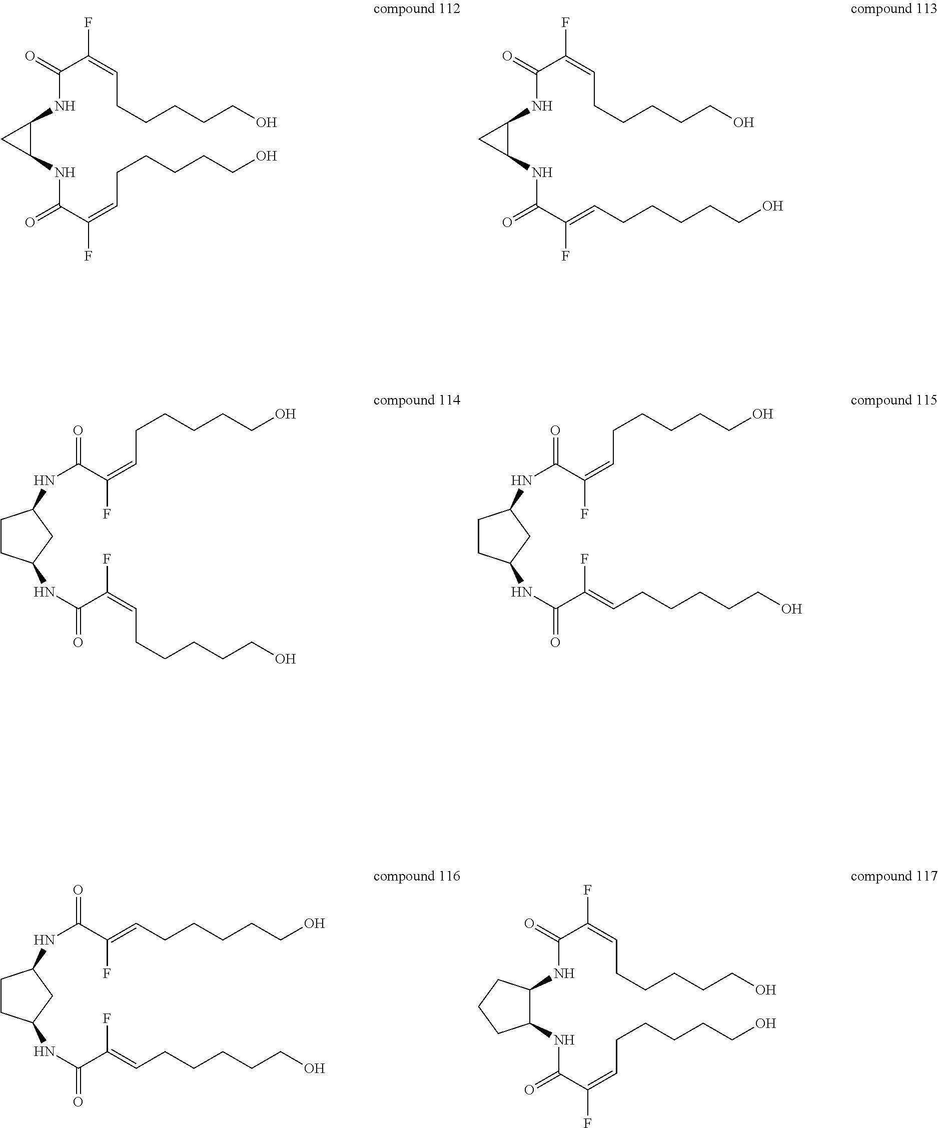

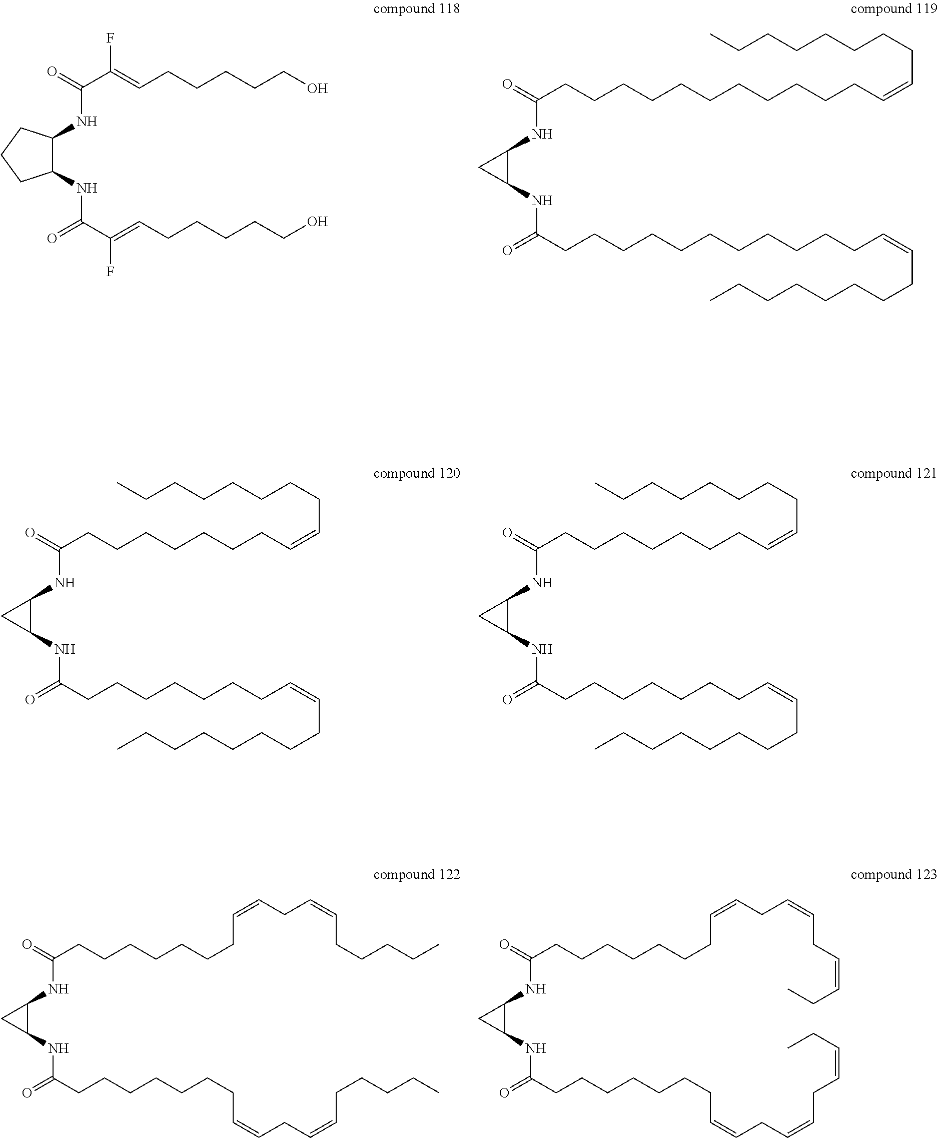

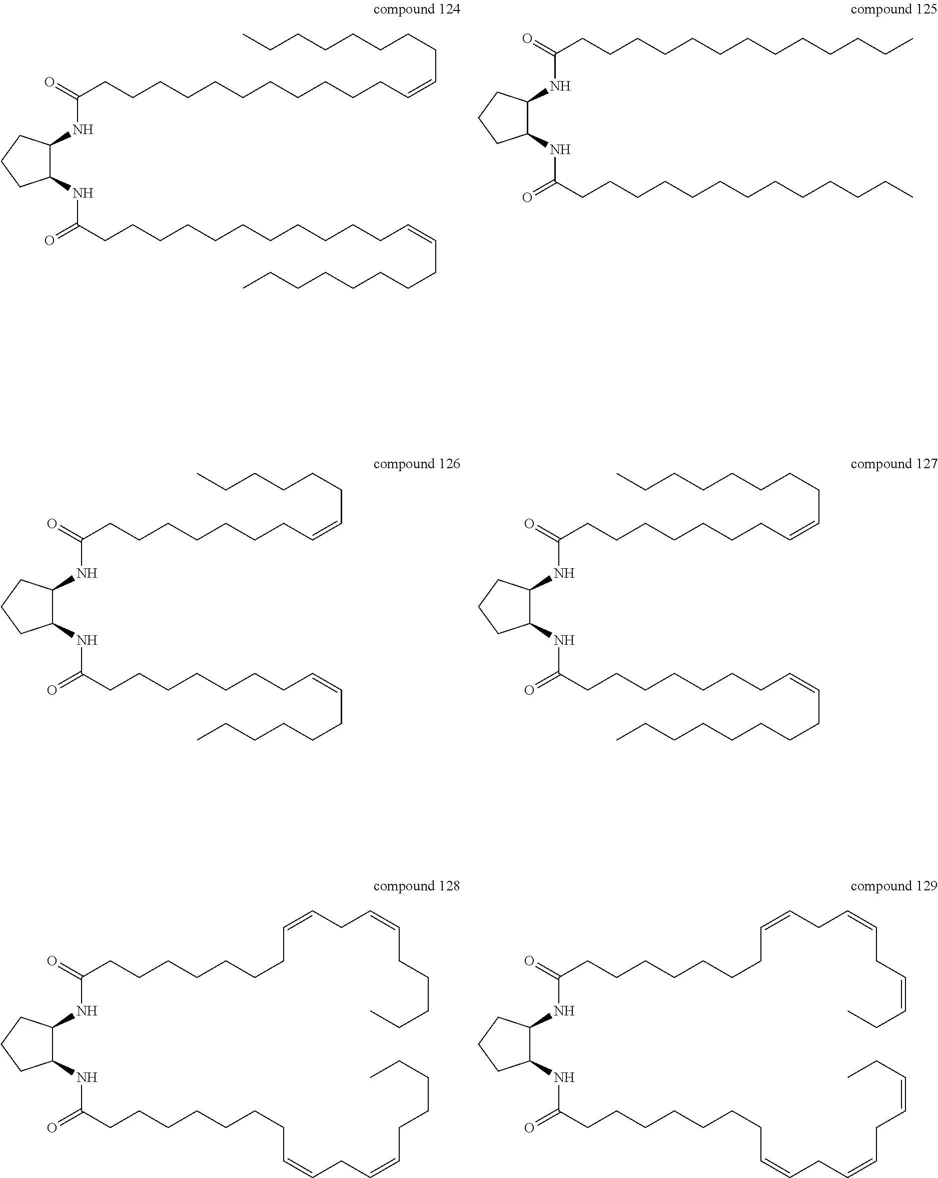

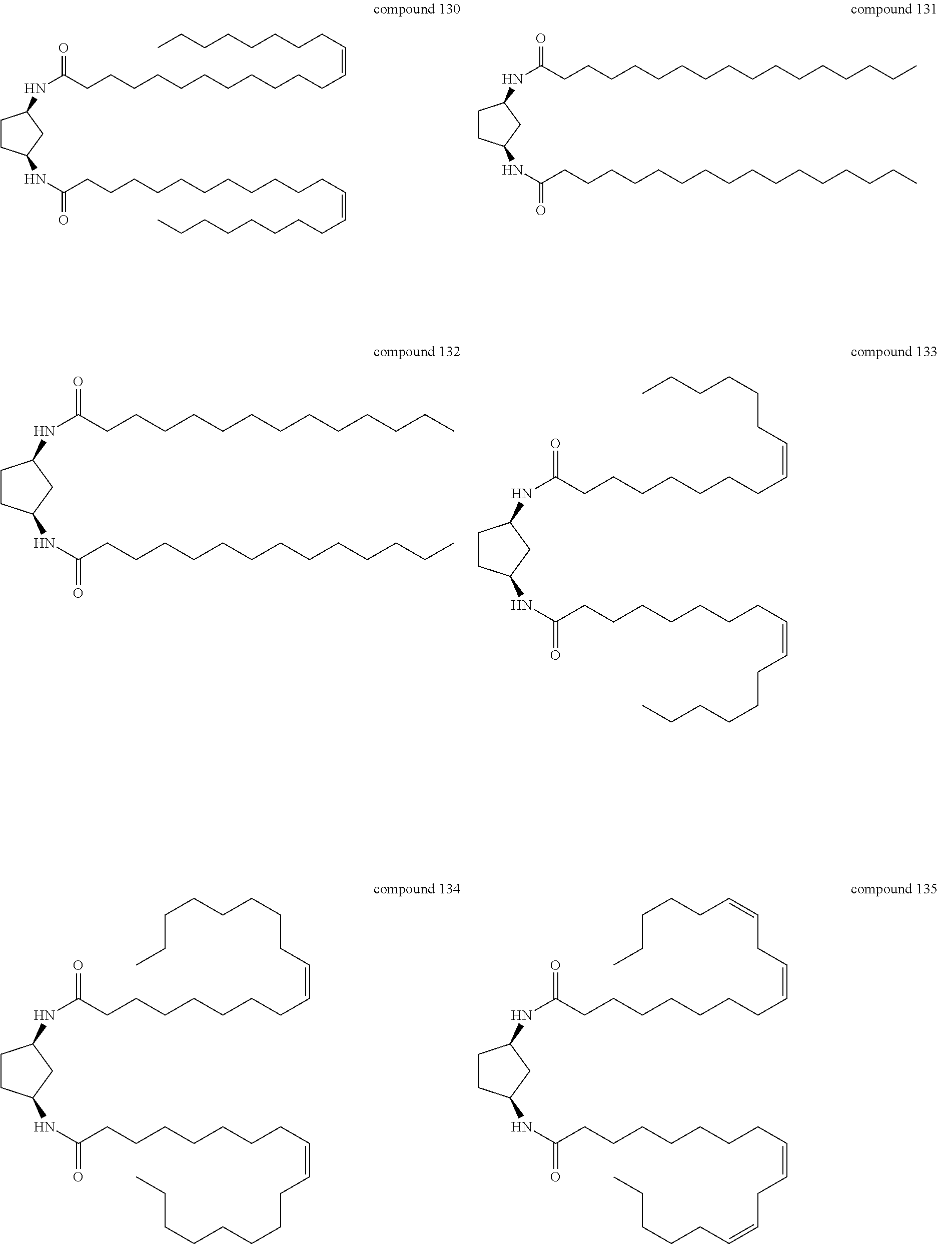

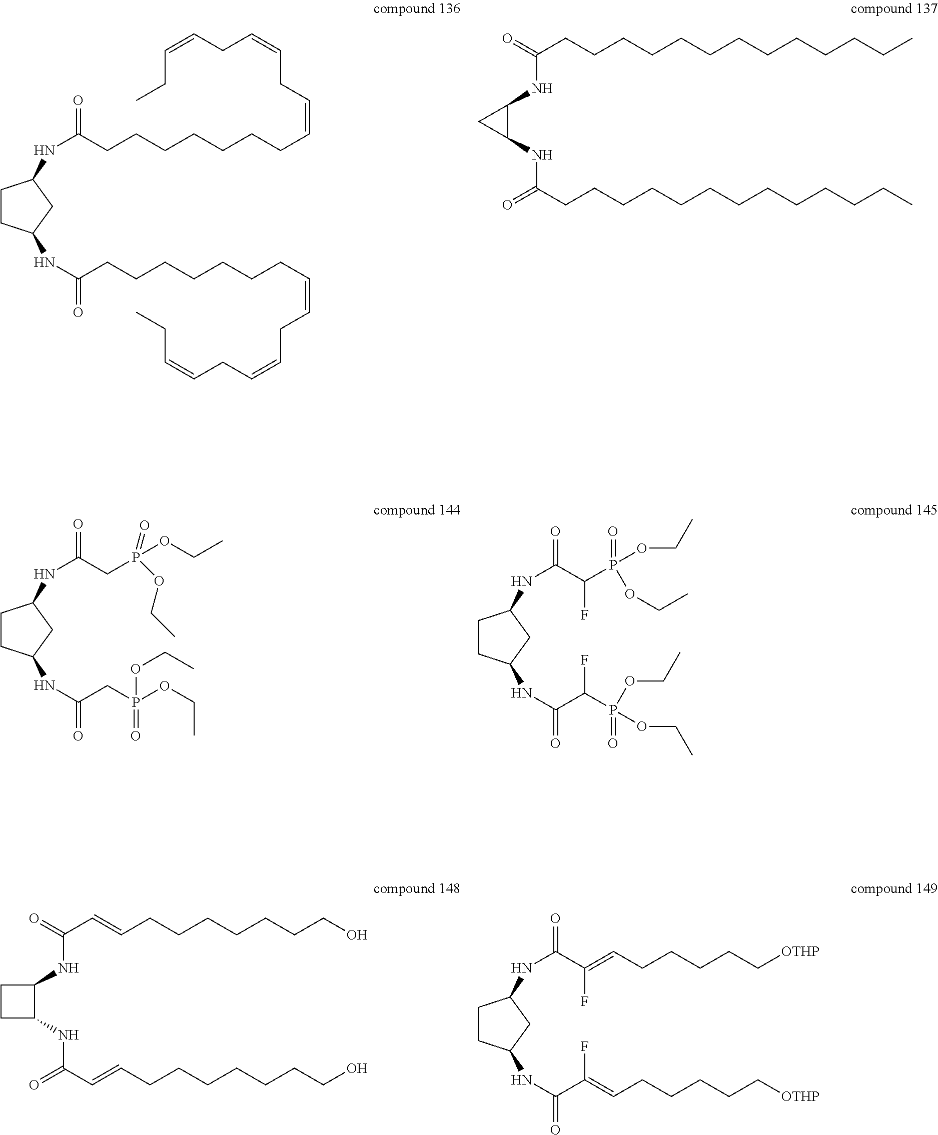

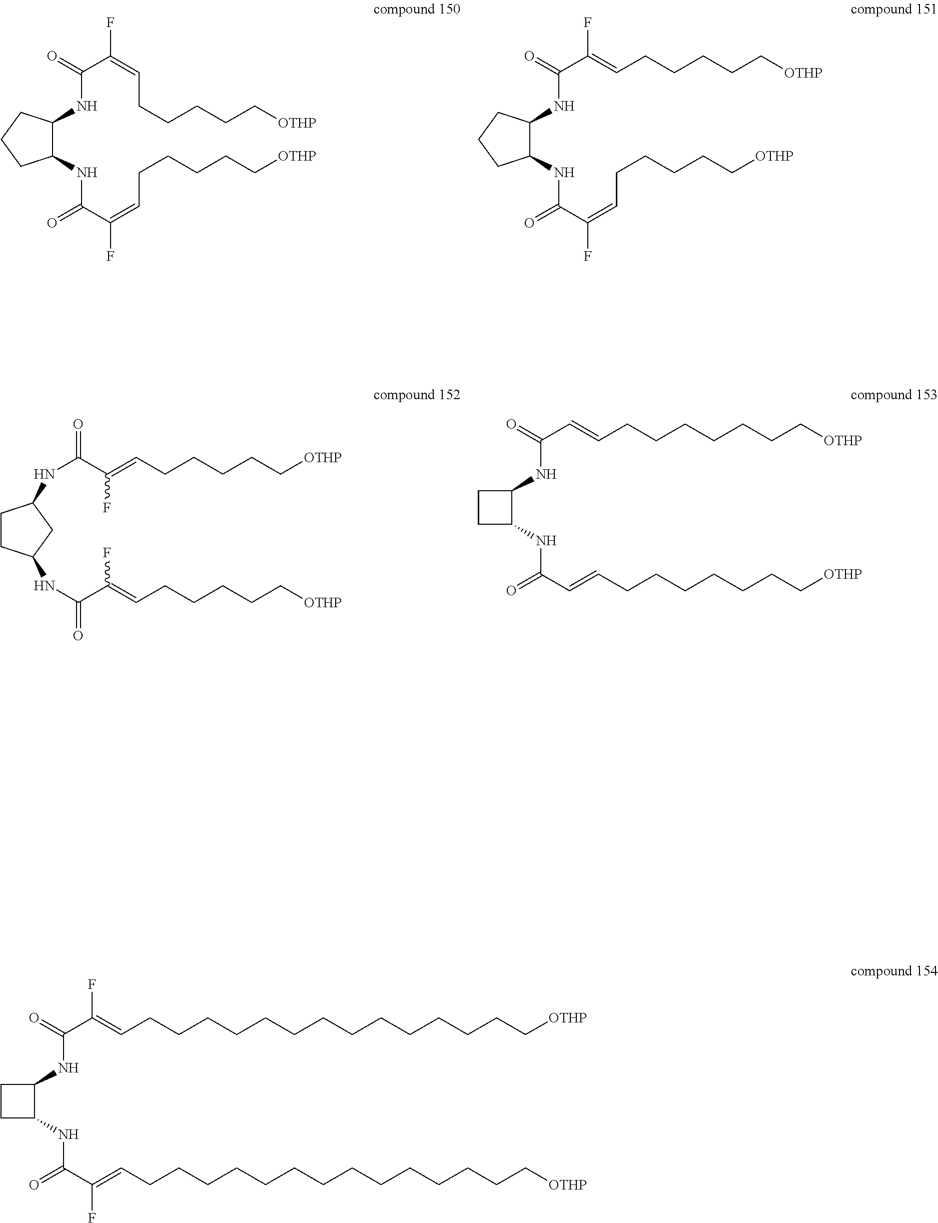

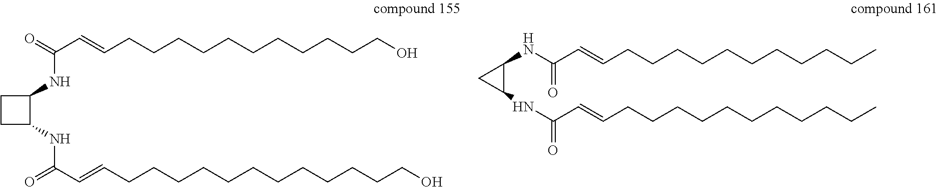

The invention further relates to a process for the preparation of compounds of Formula I, cis and trans, represented by the following formula:

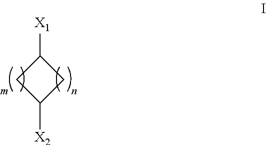

in which

m=1, 2, 3 and n=0, 1,

provided that m+n is different from 4,

X₁ and X₂ can be trans or cis relative to one another and represent, independently of one another, a group selected from

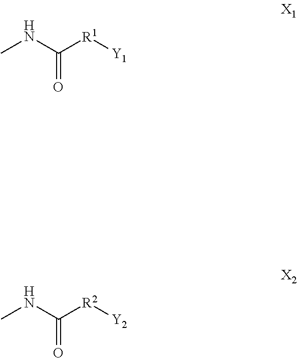

in which
[symbol] Y₁ represents
   —H,
   —OH,
   —OH optionally coupled to a glycoside compound, which can be an α- or β-furanose or an α- or β-pyranose,
   —ORₐ,
   —OCOCH₃,
   —OSi(Rₐ)₃,
   —OSitBdPh of formula

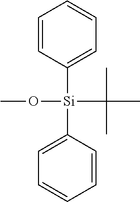 [SMILES: CO[Si](c1ccccc1)(c1ccccc1)C(C)(C)C]

—OSitBdM of formula

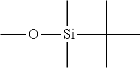

—COOH,
—COOR_{b},
—NH₂,
—NR_{c}R_{d},
—NHCORₑ,
—NHCOOR_{f},
the —OTHP group of formula

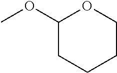

a group derived from ethylene glycol of formula

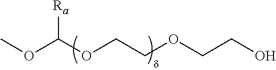

in which δ varies from 1 to 12,
a group derived from propylene glycol of formula,

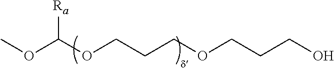

in which δ′ varies from 1 to 5,
an —O—CH(R_{z})—O-Q group, in which R_{z}, represents an alkyl or aralkyl group comprising from 1 to 30 carbon atoms, which can, but does not necessarily, contain one or more ether functions and optionally a terminal hydroxyl,
   Rₐ, R_{b}, R_{c}, R_{d} represent linear or branched alkyl groups comprising from 1 to 4 carbon atoms, optionally substituted by one or more halogen atoms, or carbon chains interrupted by oxygen or sulphur atoms, benzyl groups optionally substituted by a halogen atom, a hydroxy group, an alkoxy group comprising from 1 to 8 carbon atoms,
   Rₑ represents a linear or branched alkyl group containing from 1 to 4 carbon atoms, a phthalimido group (in this case the NH is replaced by N), a benzyl group optionally substituted by a halogen atom, a hydroxy group, an alkoxy group and in particular substituted by the methoxy group in the para position,
   R_{f} represents a linear or branched alkyl group comprising from 1 to 4 carbon atoms, optionally substituted by one or more halogen atoms, or a carbon chain interrupted by oxygen or sulphur atoms, a phenyl group, a benzyl group optionally substituted by a halogen atom, a hydroxy group, an alkoxy group and in particular substituted by the methoxy group in the para position,
the phosphonate group of formula

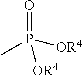

in which
R⁴ represents a linear or branched alkyl group, comprising from 1 to 6 carbon atoms, in particular methyl, ethyl, isopropyl, tert-butyl, (OR⁴)₂ optionally forming a ring between the two oxygen atoms, the (OR⁴)₂ groups in particular originating from diols such as ethane-1,2-diol, propane-1,3-diol, 2,2-dimethylpropane-1,3-diol, 2,3-dimethylbutane-2,3-diol (pinacol), 2-methylbutane-2,3-diol, 1,2-diphenylethane-1,2-diol, 2-methylpentane-2,4-diol, 1,2-dihydroxybenzene (catechol), 2,2′-azanediyldiethanol, 2,2′-(butylazanediyl)diethanol, 2,3-dihydroxysuccinic acid (tartaric acid) and its esters, or (OR⁴)₂ originates in particular from diacids such as 2,2′-(methylazanediyl)diacetic acid (mida),
[symbol] R¹ represents a linear or branched chain having from 1 to 30 carbon atoms, R¹ being saturated or unsaturated, unsubstituted or substituted by a halogen atom,
   and in the case of an unsaturation, the C═C double bond optionally being substituted by a fluorine, chlorine, or bromine atom or by a —CF₃ group,
   and in the case when R¹ only comprises a single carbon, it is selected from the groups of Formula “—CHV—”, in which V represents —H, —F, —Cl or —Br, Y₁ then being equal to the phosphonate group —P(O)(OR⁴)₂, R⁴ having the meaning given above,
      said process comprising a reaction of amide formation between a compound of Formula VII

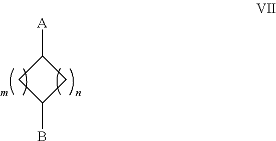

in which
m=1, 2, 3 and n=0, 1, provided that m+n is different from 4,
A=—NH₂, —NH—CO—R¹—Y₁
B=—NH₂, —NH—CO—R¹—Y₁
   provided that if A=—NH—CO—R¹—Y₁ then B=—NH₂,
   and a compound of general formula VIII

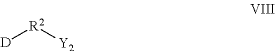

in which

Y₂ has the same meaning as Y₁,

R² has the same meaning as R¹,

Y₁ and Y₂ being able to be equal or different,
R¹ and R² being able to be equal or different,

D=—CO—R⁵

R⁵ representing
a hydroxy group —OH,
an alkoxy group —OR⁶, R⁶ representing a linear or branched alkyl chain comprising from 1 to 8 carbon atoms,
a chlorine atom —Cl,
an acyloxy group —O—CO—R⁷, R⁷ representing a linear or branched alkyl chain comprising from 1 to 8 carbon atoms, or optionally being equal to —R²—Y₂, the meanings of R² and of Y₂ being those defined above,
a group derived from benzotriazole —OR⁸, of formula

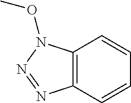

in particular derived from
HATU (2-(1H-7-azabenzotriazol-1-yl)-1,1,3,3-tetramethyluronium hexafluorophosphate methanaminium),
HBTU (2-(1H-benzotriazol-1-yl)-1,1,3,3-tetramethyluronium hexafluorophosphate,
HOBt (1-hydroxybenzotriazole),
BOP (benzotriazol-1-yl-oxy-tris-(dimethylamino)-phosphonium hexafluorophosphate),
PyBOP (benzotriazol-1-yl-oxy-tris-(dimethylamino)-phosphonium hexafluorophosphate),
   a group derived from a carbodiimide, of formula

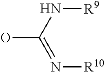

in which
R⁹ and R¹⁰, different or equal, represent an alkyl group comprising from 1 to 10 carbon atoms, linear, branched or cyclic, optionally substituted by an amino group, in particular cyclohexyl, isopropyl, ethyl, dimethylpropylamino,
said carbodiimide in particular being selected from the following compounds
DCC(N,N′-dicyclohexylcarbodiimide),
EDCI (1-ethyl-3-(3-dimethylaminopropyl)carbodiimide),
DIC (N,N′-diisopropylcarbodiimide),
   said amide formation reaction making it possible to obtain the compounds of Formula I represented above.
   If R⁵ is a hydroxy group —OH, then compound VIII is a carboxylic acid.
   If R⁵ is an alkoxy group —OR⁶, then compound VIII is an ester.
   If R⁵ is a chlorine atom —Cl, then compound VIII is an acid chloride.
   If R⁵ is an acyloxy group —O—CO—R⁷, then compound VIII is an acid anhydride, symmetrical if R⁷ is equal to —R²—Y₂, otherwise mixed.
   If R⁵ is a group derived from benzotriazole —OR⁸, then compound VIII is an activated ester.
   If R⁵ is a group derived from carbodiimide, then compound VIII is an O-acylisourea.
[symbol] “If A=B=—NH₂ then D=—CO—R⁵”: compound VII bears two —NH₂ groups, then two equivalents of the carboxylic acid of general formula VIII will be used. Two couplings therefore take place during the same reaction stage, making it possible to obtain the side chains bearing the amide function. The molecule obtained is symmetrical, the chains having the same parts R¹ and Y₁, the meanings of R¹ and Y₁ being stated above.
[symbol] “If A≠B with A=—NH₂ and B=—NH—CO—R¹—Y₁, then D=—CO—R⁵”: compound VII already has a side chain obtained by a preceding amide formation. In the course of the last reaction stage of the process, the second amide formation is then carried out. The compound obtained then has two amide chains attached to the ring, differing by the respective natures of R¹ and R² and of Y₁ and Y₂. The meanings of R¹, R², Y₁ and Y₂ were defined above. These compounds are described as asymmetrical as the side chains are different. In this process, two amide formations are therefore carried out but in two different stages so as to be able to obtain side chains of different types.

The invention relates to a process for the preparation of the compounds of Formula I_{A} and I_{B}, cis and trans, represented by the formulae shown below:

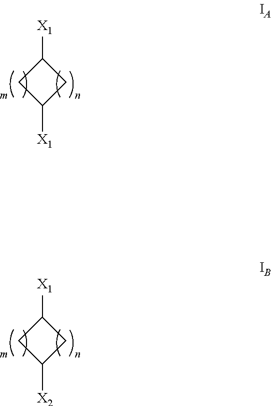

in which
X₁ and X₂ have the meanings given above,
which comprises an amide formation between a compound of Formula VII shown below:

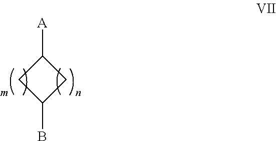

in which

m=1, 2, 3 and n=0, 1, provided that m+n is different from 4,

A and B are such that:
[symbol] A=B=—NH₂,
[symbol] or A=—NH₂ and B=—NH—CO—R¹—Y₁,
   and a compound of Formula VIII_{A}

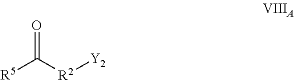

in which
R², R⁵ and Y₂ have the meanings given above,
Y₁ and Y₂ being able to be equal or different,
R¹ and R² being able to be equal or different,
said process making it possible to obtain the compounds of Formula I_{A} and I_{B} represented above.
The symmetrical and asymmetrical compounds defined above are included. If the target product bears two X₁ groups, it is symmetrical. If the target product bears an X₁ group and an X₂ group with X₁ and X₂ different, it is asymmetrical. The formulae of X₁ and of X₂ are shown below:

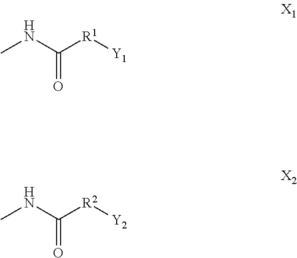

[symbol] If “A=B=—NH₂”, then two amide formation reactions take place in the same reaction stage with two equivalents of the compound of Formula VIII_{A}. The compound is symmetrical.
[symbol] If “A=—NH₂ and B=—NH—CO—R¹—Y₁”, then the second amide formation takes place with one equivalent of the compound of Formula VIII_{A}. The compound is asymmetrical.

The invention relates in particular to a process for the preparation of the symmetrical compounds of Formula II_{A}, cis and trans, shown below:

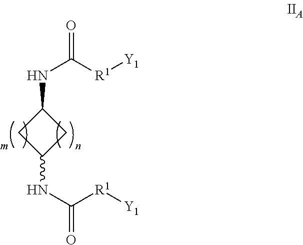

in which

m=1, 2, 3 and n=0.1, provided that m+n is different from 4,

R¹, Y₁ have the meanings given above,

comprising coupling between a cis or trans diamine of Formula VII_{A} shown below:

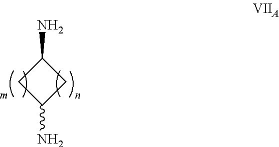

in which
m and n have the meanings given above,
and a compound of Formula VIII_{A}

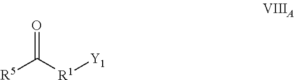

R¹, R⁵ and Y₁ having the meanings given above,
said process making it possible to obtain the compounds of Formula II_{A} represented above.
The amide formation is carried out in a standard manner, in particular
by reaction between a carboxylic acid and an amine (R⁵=—OH),
by reaction between an activated form of the acid and an amine, and the activated form can be an acid chloride (R⁵=—Cl), an anhydride (R⁵=—O—CO—R⁷),
by reaction between an activated form of the ester, said activation being obtained from a benzotriazole derivative or from a carbodiimide derivative.
   This coupling is carried out in cis or trans series and is represented by the following chemical equation:

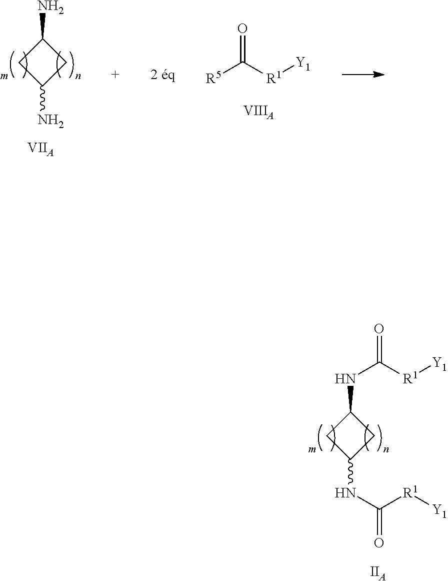

Therefore one stage is sufficient for preparing the compounds II_{A}.

Advantageously, the invention relates to a process for the preparation of the symmetrical compounds of Formula II_{A cis} shown below

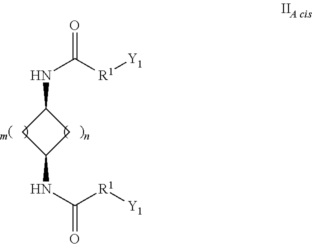

in which

m=1, 2, 3 and n=0.1, provided that m+n is different from 4,

R¹ and Y₁ have the meanings given above,

said process comprising coupling between a diamine of Formula VII_{A cis} shown below:

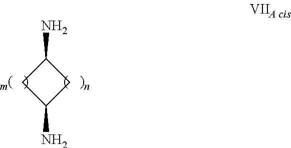

in which
m and n have the meanings given above,
and a compound of Formula VIII_{A}

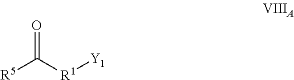

R⁵ having the meanings given above and in particular being equal to —OH,
R¹ and Y₁ having the meanings given above,
said process making it possible to obtain the compounds of Formula II_{A cis} represented above.

Particularly advantageously, the invention relates to a process for the preparation of the symmetrical compounds of Formula VI_{F cis} represented below

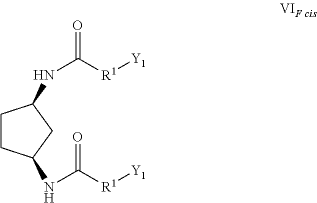

in which
R¹ and Y₁ have the meanings given above,
said process comprising coupling between cis-1,3-diaminocyclopentane of the formula shown below

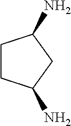

and a compound of Formula VIII_{A}

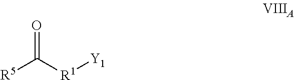

R⁵ having the meanings given above and in particular being equal to —OH,
R¹ and Y₁ having the meanings given above,
said process making it possible to obtain the compounds of Formula VI_{F cis} represented above.

The invention relates in particular to a process for the preparation of compound 30 of the formula shown below

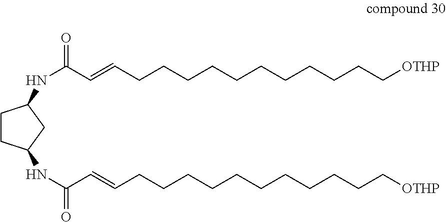

in which —OTHP is the group of formula,

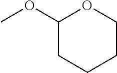

said process comprising coupling between cis-1,3-diaminocyclopentane of the formula shown below

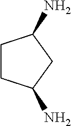

and the acid of the formula shown below

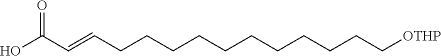

in which —OTHP has the meaning designated above,
said process making it possible to obtain compound 30 of the formula shown above.

The invention also relates in particular to a process for the preparation of compound 152 of the formula shown below

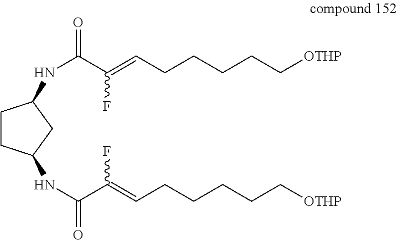

in which —OTHP has the meaning designated above,
said process comprising coupling between cis-1,3-diaminocyclopentane of the formula shown below:

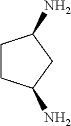

and the acid of the formula shown below

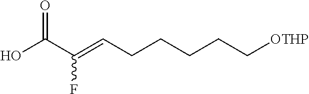

in which —OTHP has the meaning designated above,
said process making it possible to obtain compound 152 of the formula shown above.

The invention also relates to a process for the preparation of the cis and trans asymmetrical compounds of Formula II_{B} shown below:

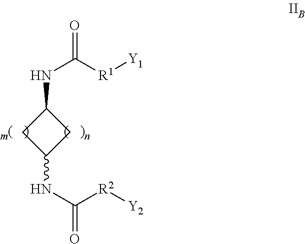

in which
m=1, 2, 3 and n=0, 1, provided that m+n is different from 4,
R¹, R², Y₁ and Y₂ have the meanings given above,

provided that R¹ and R² are different from one another,
Y₁ and Y₂ are identical or different,
   which comprises a reaction between an aminoamide of Formula VII_{I} represented by the formula shown below:

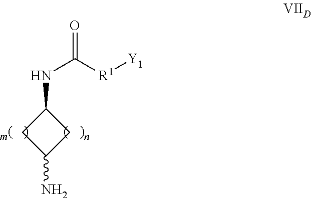

in which
R¹, Y₁, m and n have the meanings given above,
and a compound of Formula VIII_{A}

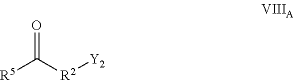

in which
R², R⁵ and Y₂ have the meanings given above,
said process making it possible to obtain the compounds of Formula II_{B} represented above.
Preparation of the asymmetrical molecules requires four reaction stages. The last stage is shown in the following diagram: it is the second amide formation, carried out in cis or trans series:

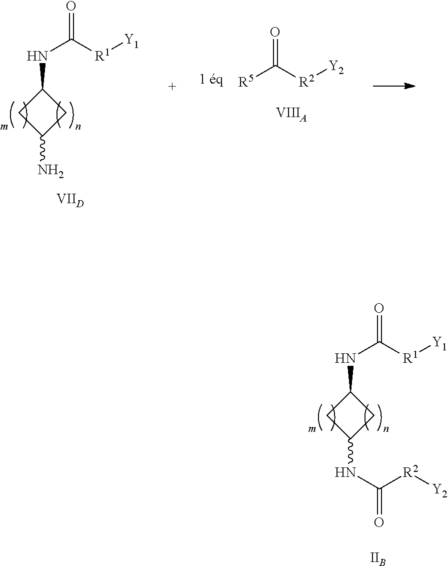

The compound obtained then has two amide chains attached to the ring by the nitrogen atom, but different in the respective natures of R¹ and R² and/or of Y₁ and Y₂. “R¹—Y₁” is supplied during the first amide formation whereas “R²—Y₂” is supplied during the second amide formation. By proceeding in this way, it is possible to prepare asymmetrical compounds.

The invention relates in particular to a process for the preparation of compound VII_{D} represented by the formula shown below:

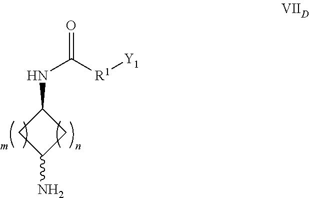

said compound VII_{D} being obtained by deprotection of the amine function of compound IX represented below

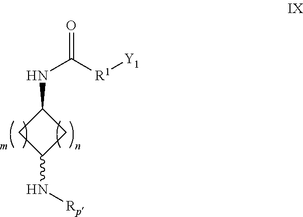

in which
Rp′ is a protective group of the amines selected from:
   —CORₑ, in which Rₑ represents a linear or branched alkyl group containing from 1 to 4 carbon atoms, a phthalimido group (in this case the NH is replaced by N), a benzyl group optionally substituted by a halogen atom, a hydroxy group, an alkoxy group and in particular substituted by the methoxy group in the para position,
   —COOR_{f}, in which R_{f} represents a linear or branched alkyl group comprising from 1 to 4 carbon atoms, optionally substituted by one or more halogen atoms, more particularly methyl, ethyl, propyl, tert-butyl, or a carbon chain interrupted by oxygen or sulphur atoms, a phenyl group, a benzyl group or derivatives thereof, optionally substituted by a halogen atom, a hydroxy group, an alkoxy group and in particular substituted by the methoxy group in the para position,
   the benzyl group or derivatives thereof,
m=1, 2, 3 and n=0, 1, provided that m+n is different from 4,
R¹, Y₁ have the meanings given above,
   said process making it possible to obtain the compounds of Formula VII_{D} represented above.
   This stage is the last one for obtaining II_{B}. In order to carry out the two separate amide formations that lead to the asymmetrical compounds II_{B}, an amine function of the cyclic compound was protected beforehand in the form “—NH-Rp′”. The following equation makes it possible to represent the deprotection stage, making the second amine function usable once again for a coupling of the peptide type:

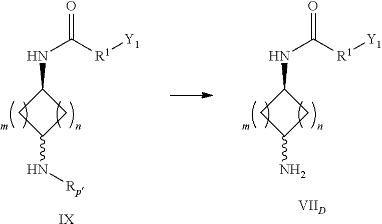

The invention relates in particular to a process for the preparation of compound IX represented by the formula shown below:

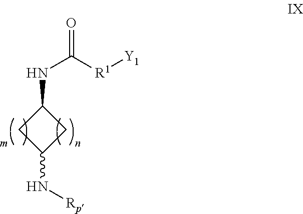

said compound IX being obtained by monoacylation between the diamine X, one amine function of which is blocked by a protective group

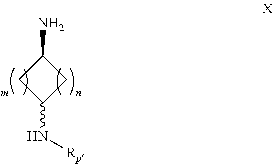

in which
Rp′ is a protective group of the amines selected from:
   —CORₑ, in which Rₑ represents a linear or branched alkyl group containing from 1 to 4 carbon atoms, a phthalimido group (in this case the NH is replaced by N), a benzyl group optionally substituted by a halogen atom, a hydroxy group, an alkoxy group and in particular substituted by the methoxy group in the para position,
   —COOR_{f}, in which R_{f} represents a linear or branched alkyl group comprising from 1 to 4 carbon atoms, optionally substituted by one or more halogen atoms, more particularly methyl, ethyl, propyl, tert-butyl, or a carbon chain interrupted by oxygen or sulphur atoms, a phenyl group, a benzyl group or derivatives thereof, optionally substituted by a halogen atom, a hydroxy group, an alkoxy group and in particular substituted by the methoxy group in the para position,
   the benzyl group or derivatives thereof,
m=1, 2, 3 and n=0, 1, provided that m+n is different from 4,
R¹, Y₁ have the meanings given above,
   and a compound of Formula VIII_{A} represented by the formula shown below:

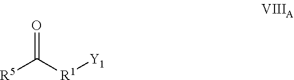

in which
R¹, R⁵ and Y₁ have the meanings given above,
said process making it possible to obtain the compounds of Formula IX represented above.
This reaction is the first one in the process for the preparation of the asymmetrical compounds. Since an amine function is blocked, the first coupling makes it possible to obtain compound IX. The chemical equation of this coupling is shown below:

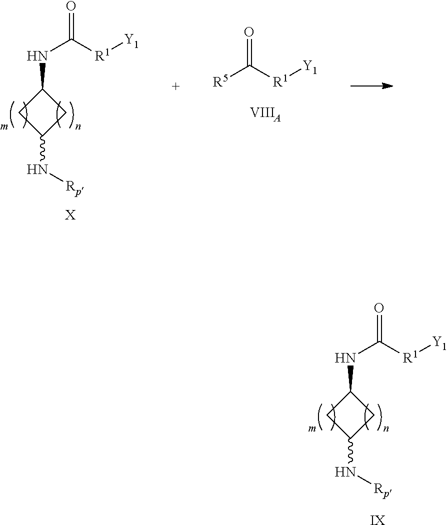

The first side chain is thus attached to the ring.

The invention relates in particular to a process for the preparation of the compound X represented by the formula shown below:

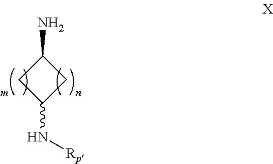

said compound X being obtained by protection of the diamine of Formula VII_{A} shown below:

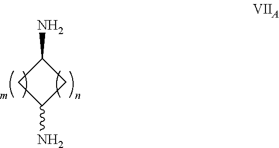

in which
m=1, 2, 3 and n=0, 1, provided that m+n is different from 4,
said process making it possible to obtain the compounds of Formula X represented above.
A single amine function of compound VII_{A} is protected so as to be able to carry out the first amide formation. Protection is carried out by conversion to an amide or carbamate function. It is represented by the following equation:

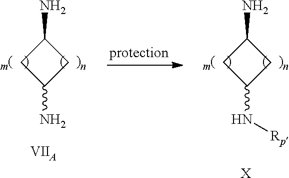

The invention relates in particular to a process for the preparation of the cis and trans compounds of Formula II_{B} shown below:

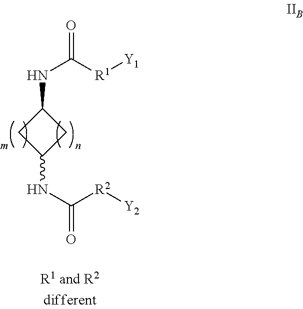

in which
m=1, 2, 3 and n=0, 1, provided that m+n is 4,
R¹, R², Y₁ and Y₂ have the meanings given above,

provided that R¹ and R² are different from one another,
Y₁ and Y₂ being identical or different,
[symbol] process comprising a 1st stage that consists of protecting one of the amino groups of the diamine of Formula VII_{A} shown below:

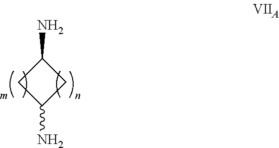

m and n having the meanings given above,
to obtain compound X of the following formula:

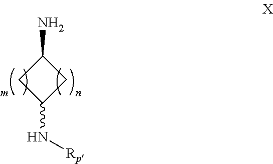

in which
m and n have the meanings given above,
Rp′ is a protective group of the amines selected from:
   —CORₑ, in which Rₑ represents a linear or branched alkyl group containing from 1 to 4 carbon atoms, a phthalimido group (in this case the NH is replaced by N), a benzyl group optionally substituted by a halogen atom, a hydroxy group, an alkoxy group and in particular substituted by the methoxy group in the para position,
   —COOR_{f}, in which R_{f} represents a linear or branched alkyl group comprising from 1 to 4 carbon atoms, optionally substituted by one or more halogen atoms, more particularly methyl, ethyl, propyl, tert-butyl, or a carbon chain interrupted by oxygen or sulphur atoms, a phenyl group, a benzyl group or derivatives thereof, optionally substituted by a halogen atom, a hydroxy group, an alkoxy group and in particular substituted by the methoxy group in the para position,
   the benzyl group or derivatives thereof,
[symbol] process comprising a second stage that consists of carrying out an amide formation between compound X represented above and a compound of Formula VIII_{A} represented by the formula shown below:

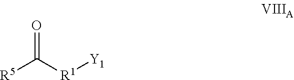

in which
R¹, R⁵ and Y₁ have the meanings given above,
to obtain the monomeric compound IX having the following formula:

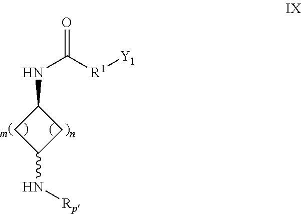

in which
m, n, R¹, Y₁ and R_{p′} have the meanings given above,
[symbol] process comprising a third stage that consists of carrying out a deprotection of the amino group of compound IX to obtain the compound of Formula VII_{D} shown below:

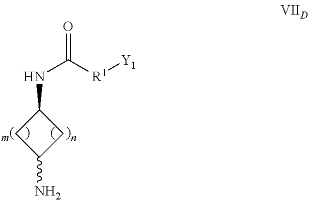

in which
m, n, R¹, Y₁ have the meanings given above,
[symbol] process comprising a fourth stage that consists of carrying out an amide formation between compound VII_{D} above and compound VIII_{A} of the following formula:

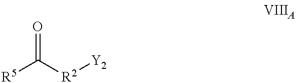

provided that R¹ and R² are different from one another,
to obtain the target compound II_{B}:

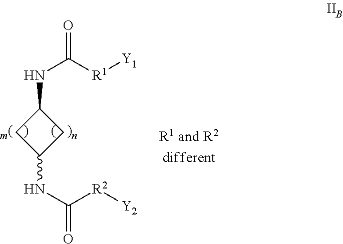

m, n, R¹, R², Y₁, Y₂ having the meanings given above.
The process for the preparation of the family of asymmetrical compounds therefore involves four stages, carried out in cis or trans series.
The first stage consists of protecting an amine function of compound VII_{A}.
The second stage consists of carrying out the first amide formation by reaction with one equivalent of compound VIII_{A}.
The third stage consists of deprotecting the second amine function, making it available for the fourth and last stage.
The fourth stage consists of carrying out the second amide formation.
   This reaction sequence is shown below:

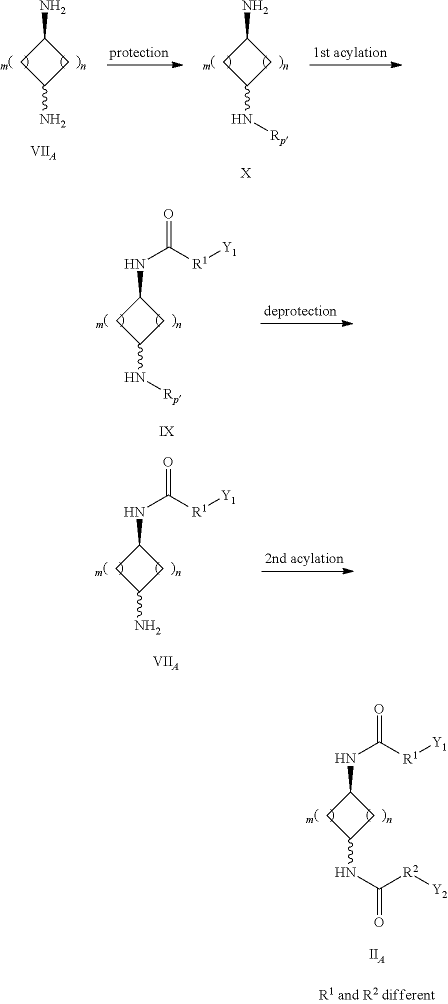

The side chains are different as they originate from carboxylic acids or differently substituted derivatives, R⁵—CO—R¹—Y₁ and R⁵—CO—R²—Y₂.

The invention also relates to a process for the specific preparation of the compounds of Formula II_{C} shown below:

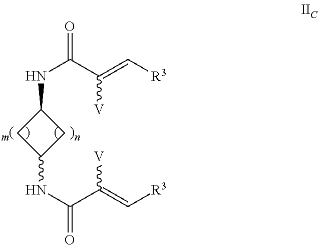

in which
m=1, 2, 3 and n=0, 1, provided that m+n is different from 4,
V=H, F, Cl or Br,
R³ represents an unbranched linear alkyl chain, saturated or unsaturated, comprising from 5 to 28 carbon atoms, terminated by a hydrogen, an —OH group, or a protected form of the latter, an —NH₂ group or a protected form of the latter, in particular —NHBoc,
   said compounds II_{C} being obtained by Wittig-Horner reaction between an aldehyde of general formula XVII

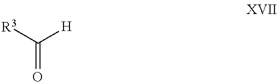

R₃ having the meaning given above,
and a phosphonoacetamide of general formula XVIII

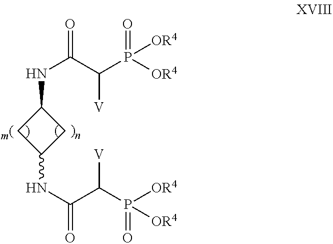

in which
m, n and V have the meanings given above,
R⁴ represents a linear or branched alkyl group, comprising from 1 to 6 carbon atoms, in particular methyl, ethyl, isopropyl, tert-butyl, (OR⁴)₂ optionally forming a ring between the two oxygen atoms, the (OR⁴)₂ groups in particular originating from diols such as ethane-1,2-diol, propane-1,3-diol, 2,2-dimethylpropane-1,3-diol, 2,3-dimethylbutane-2,3-diol (pinacol), 2-methylbutane-2,3-diol, 1,2-diphenylethane-1,2-diol, 2-methylpentane-2,4-diol, 1,2-dihydroxybenzene (catechol), 2,2′-azanediyldiethanol, 2,2′-(butylazanediyl)diethanol, 2,3-dihydroxysuccinic acid (tartaric acid) and its esters, or (OR⁴)₂ originates in particular from diacids such as 2,2′-(methylazanediyl)diacetic acid (mida), in particular methyl, ethyl, isopropyl, tert-butyl, said process making it possible to obtain the compounds of Formula II_{C} represented above. This second process for the preparation of cyclic diamides involves a reaction of the Wittig-Horner type between a phosphonoacetamide represented by Formula XVIII and an aldehyde of Formula XVII.
   This process represents a second method for the preparation of the symmetrical compounds, the side chains being identical. The cyclic unit is already present on the phosphonoacetamide, a compound that is stable and easy to handle. This reaction makes it possible to create unsaturated chains bearing a double bond conjugated with the carbonyl of the amide. Moreover, the phosphorus-containing derivative can carry a halogen, V=F, Cl, Br, which makes it possible to obtain halogenated compounds II_{C}.
   The equation of this reaction is shown below:

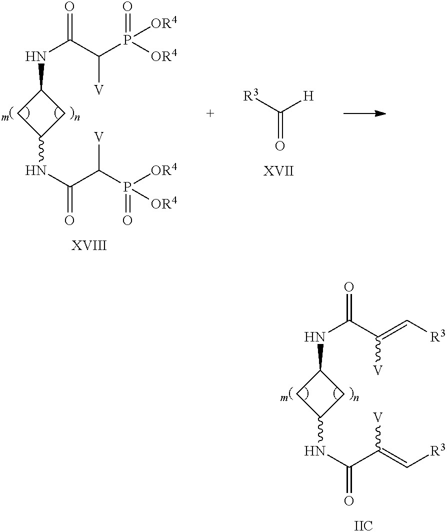

The invention also relates to a process for the preparation of the phosphonoacetamide XVIII represented by the formula shown below

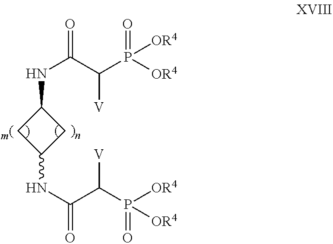

in which
m=1, 2, 3 and n=0, 1, provided that m+n is different from 4,
V=H, F, Cl or Br,
R⁴ represents a linear or branched alkyl group, comprising from 1 to 6 carbon atoms, in particular methyl, ethyl, isopropyl, tert-butyl, (OR⁴)₂ optionally forming a ring between the two oxygen atoms, the (OR⁴)₂ groups in particular originating from diols such as ethane-1,2-diol, propane-1,3-diol, 2,2-dimethylpropane-1,3-diol, 2,3-dimethylbutane-2,3-diol (pinacol), 2-methylbutane-2,3-diol, 1,2-diphenylethane-1,2-diol, 2-methylpentane-2,4-diol, 1,2-dihydroxybenzene (catechol), 2,2′-azanediyldiethanol, 2,2′-(butylazanediyl)diethanol, 2,3-dihydroxysuccinic acid (tartaric acid) and its esters, or (OR⁴)₂ originates in particular from diacids such as 2,2′-(methylazanediyl)diacetic acid (mida), in particular methyl, ethyl, isopropyl, tert-butyl, said compound XVIII being obtained by an amide formation between the diamine of general Formula VII_{A} shown below:

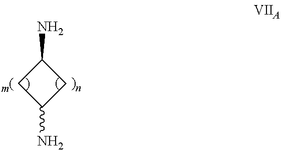

m and n having the meanings given above,
and the phosphorylated carboxylic acid of Formula VIII_{C}

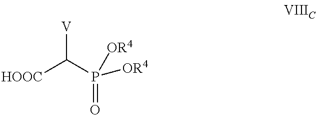

in which
V and R⁴ have the meanings given above,
said process making it possible to obtain the compounds of Formula XVIII represented above.
This reaction makes it possible to prepare the phosphonoacetamide represented by Formula XVIII by amide formation between the cyclic diamine of Formula VII_{A} and a phosphonoacetic acid. It is possible to work in halogenated series, V then representing fluorine, chlorine or bromine, carried by the carbon in the α position to the carboxyl group.
The reaction is carried out under standard amide formation conditions.
It is represented by the following equation:

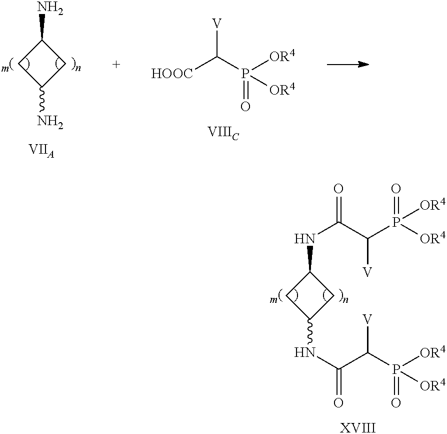

The product obtained is thus a phosphonic amide that can be used in a reaction of the Wittig-Horner type, as described above. It is obtained with a yield of the order of 95%, after purification by silica chromatography.

The invention also relates to a process for the preparation of the compounds of Formula II_{C} shown below:

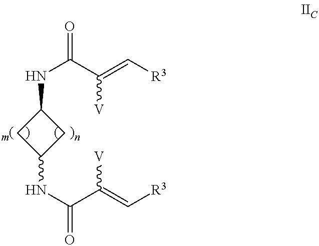

in which
m=1, 2, 3 and n=0, 1, provided that m+n is different from 4,
V=H, F, Cl or Br,
R³ represents an unbranched linear alkyl chain, saturated or unsaturated, comprising from 5 to 28 carbon atoms, terminated by a hydrogen, an —OH group, or a protected form of the latter, an —NH₂ group or a protected form of the latter, in particular —NHBoc,
[symbol] process comprising a first stage that consists of carrying out a reaction between the diamine of general formula VII_{A} shown below:

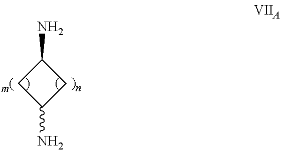

m and n having the meanings given above,
and the phosphorylated carboxylic acid of Formula VIII_{C}

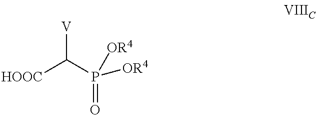

in which
V=H, F, Cl or Br,
R⁴ represents a linear or branched alkyl group, comprising from 1 to 6 carbon atoms, in particular methyl, ethyl, isopropyl, tert-butyl, (OR⁴)₂ optionally forming a ring between the two oxygen atoms, the (OR⁴)₂ groups in particular originating from diols such as ethane-1,2-diol, propane-1,3-diol, 2,2-dimethylpropane-1,3-diol, 2,3-dimethylbutane-2,3-diol (pinacol), 2-methylbutane-2,3-diol, 1,2-diphenylethane-1,2-diol, 2-methylpentane-2,4-diol, 1,2-dihydroxybenzene (catechol), 2,2′-azanediyldiethanol, 2,2′-(butylazanediyl)diethanol, 2,3-dihydroxysuccinic acid (tartaric acid) and its esters, or (OR⁴)₂ originates in particular from diacids such as 2,2′-(methylazanediyl)diacetic acid (mida), in particular methyl, ethyl, isopropyl, tert-butyl,
[symbol] process comprising a second stage that consists of carrying out a Wittig-Horner reaction between compound XVIII represented above and an aldehyde of Formula XVII represented by the following formula:

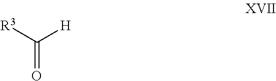

in which
R³ has the meanings given above,
said process making it possible to obtain the compounds of Formula II_{C} represented above.
A second process is described for preparing the symmetrical compounds.
The first stage consists of carrying out a reaction between a cyclic diamine and an acid bearing a phosphonic ester function. The groups —NH₂ are thus converted to phosphonoacetamides, which are compounds that are stable and easy to handle.
In the second stage, the phosphonoacetamide previously obtained is used in a reaction of the Wittig-Horner type with an aldehyde, which leads to the replacement of the phosphonate group —P(O)(OR⁴) with a carbon chain with formation of a C═C double bond optionally substituted by a halogen atom, which can be fluorine, chlorine or bromine
   This sequence in two stages is represented by the following two equations:

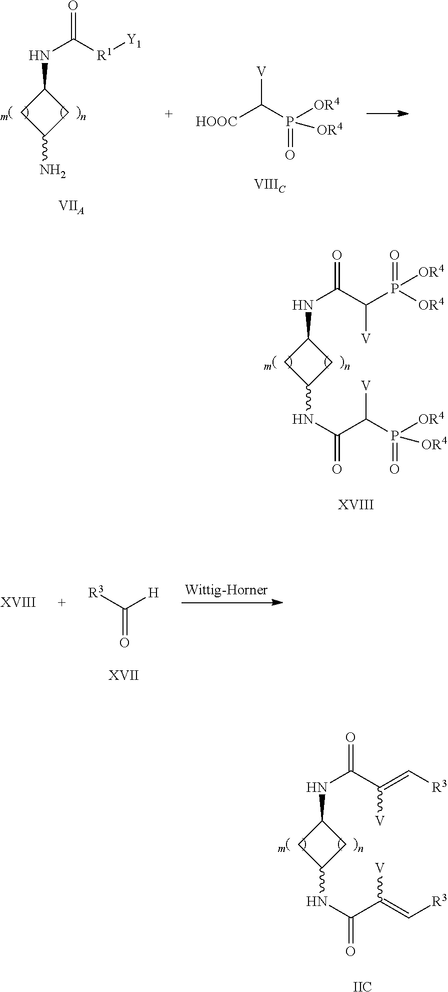

The compounds of Formula II_{C} are obtained in two stages with an excellent overall yield.

Another aspect of the invention consists of the pharmaceutical composition containing, as active ingredient, at least one of the compounds of Formula I, and in particular containing, as active ingredient, compound 30 of formula

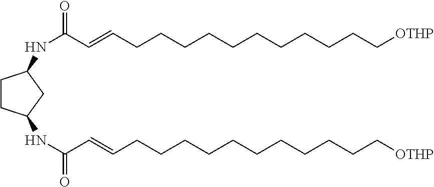

and/or compound 152 of formula

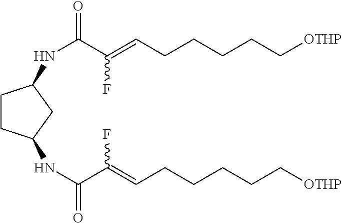

together with a pharmaceutically acceptable vehicle.
Owing to their pharmacological properties, the compounds according to the invention are used in therapeutics as skin depigmenting agents, anti-ageing agents, tensing agents, anti-inflammatory agents.
For these purposes, they will be used in the form of pharmaceutical compositions containing, as active ingredient, at least one of the compounds of general formula I in combination with or mixed with an excipient or an inert, non-toxic, and pharmaceutically acceptable vehicle.
For therapeutic use, they will be presented in one of the pharmaceutical forms suitable administration by oral or topical route.
In this connection, we may mention plain or coated tablets, sugar-coated tablets, gelatin capsules, powders, as well as creams, ointments, lotions, emulsions, sprays, serums, milks.

Another aspect of the invention consists of the pharmaceutical composition containing, as active ingredient, several of the compounds of Formula I, and in particular containing, as active ingredient, several compounds including compound 30 and/or compound 152, in combination with a pharmaceutically acceptable vehicle.

It will be possible for the pharmaceutical compositions to contain mixtures of compounds of Formula I, in variable proportions.

According to a particular aspect of the invention, the pharmaceutical composition contains from 0.005% to 20 wt % of active ingredient per unit dose.

The dosage can vary depending on the pharmaceutical form and the subject's weight.

Another aspect of the invention consists of the cosmetic composition containing, as active ingredient, at least one of the compounds of Formula I, and in particular containing, as active ingredient, compound 30 of formula

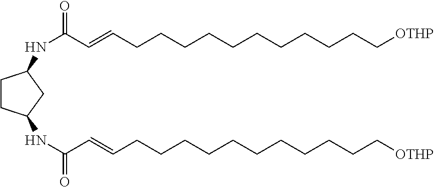

and/or compound 152 of formula

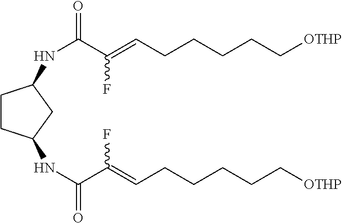

in combination with a cosmetically acceptable vehicle.
Owing to their cosmetic properties, the compounds according to the invention are used in therapeutics as skin depigmenting agents, anti-ageing agents, tensing agents, healing agents.
For these purposes, they will be used in the form of cosmetic compositions containing, as active ingredient, at least one of the compounds of general formula I in combination with or mixed with an excipient or an inert, non-toxic, and cosmetically acceptable vehicle.
For therapeutic use, they will be presented in one of the cosmetic forms suitable for administration by cutaneous route.
In this connection we may mention creams, ointments, gels, oils, serums, milks, sprays, emulsions.
The excipients that are suitable for said administrations are oils, water and alcohol as well as surfactants, additives such as preservatives, antioxidants, colorants, perfumes.

Another aspect of the invention consists of the cosmetic composition containing, as active ingredient, several of the compounds of Formula I, and in particular containing, as active ingredient, several compounds including compound 30 and/or compound 152, in combination with a cosmetically acceptable vehicle.

The cosmetic compositions will be able to contain mixtures of compounds of Formula I, in variable proportions.

According to a particular aspect of the invention, the cosmetic composition contains from 0.005% to 20 wt % of active ingredient per unit dose.

The dosage can vary depending on the form.

### EXAMPLES

The analytical techniques are as follows:

### Nuclear Magnetic Resonance

The NMR spectra were recorded at 300 MHz (Brücker spectrometer) for the proton. The chemical shifts are expressed in ppm, the residual chloroform being taken as internal reference (singlet at 7.28 ppm), or residual dimethylsulphoxide being taken as internal reference (multiplet at 2.50 ppm). The multiplicity of the signals is denoted by the following letters: s singlet, d doublet, dd doublet of doublets, t triplet, q quadruplet and m multiplet.

### Melting Point

The melting points were measured by DSC (differential scanning calorimetry) on a Mettler Toledo instrument.

### Chromatography: LCMS

LC/MS analysis corresponds to coupling of HPLC analysis and analysis by mass spectrometry. It is carried out on an Alliance Waters 2695-ZQ2000 instrument.

### HPLC (Waters ref. 2690)

Detector: DAD detector (Waters, ref.: 2996, λ=190 nm to 800 nm):

### Detector: Corona™ (ESA):

Mass detector (Waters, ref. ZQ2000): 100-1500 dalton; negative and positive ion
Temperature of HPLC oven: 40° C.
Flow: 1 mL/min
The methods used for HPLC are presented below. In the tables of analytical results, the gradient number is shown in the exponent, with the retention time.

### 1. “HCOOH ACN Grad1” Method

Column: XTerra® MS C18: 4.6 mm×150 mm, 5 μm (Waters, ref. 186000490)
Eluent A: Water (HCOOH-0.02%); Eluent B=CH₃CN) with elution gradient
Elution condition: gradient

| | HCO₂H at | | |
|---|---|---|---|
| Min. | 0.2‰ | MeCN | Curve |
| | 90 | 10 | |
| 4 | 75 | 25 | 8 |
| 5 | 65 | 35 | 6 |
| 11 | 5 | 95 | 7 |
| 14 | 5 | 95 | 7 |
| 17 | 90 | 10 | 6 |
| 20 | 90 | 10 | 6 |

### 2. “HCOOH ACN Grad7” Method

Column: XTerra® MS C18: 4.6 mm×150 mm, 5 μm (Waters ref. 186000490)
Eluent A: Water (HCOOH-0.02%); Eluent B=CH₃CN) with elution gradient
Elution condition: gradient

| | HCO₂H at | | |
|---|---|---|---|
| Min. | 0.2‰ | MeCN | Curve |
| | 50 | 50 | |
| 9 | 5 | 95 | 7 |
| 12 | 5 | 95 | 7 |
| 17 | 50 | 50 | 6 |
| 20 | 50 | 50 | 6 |

### 3. “HCOOH ACN Grad9” Method

Column: XTerra® MS C18: 4.6 mm×150 mm, 5 μm (Waters, ref. 186000490)
Eluent A: Water (HCOOH-0.02%); Eluent B=CH₃CN) with elution gradient
Elution condition: gradient

| | HCO₂H at | | |
|---|---|---|---|
| Min. | 0.2‰ | MeCN | Curve |
| | 40 | 60 | |
| 10 | 0 | 100 | 7 |
| 14 | 0 | 100 | 7 |
| 17 | 40 | 60 | 6 |
| 20 | 40 | 60 | 6 |

### 4. “HCOOH ACN Grad11” Method

Column: XTerra® MS C18: 4.6 mm×150 mm, 5 μm (Waters, ref. 186000490)
Eluent A: Water (HCOOH-0.02%); Eluent B=CH₃CN) with elution gradient
Elution condition: gradient

| | HCO₂H at | | |
|---|---|---|---|
| Min. | 0.2‰ | MeCN | Curve |
| | 30 | 70 | |
| 7 | 0 | 100 | 7 |
| 15 | 0 | 100 | 7 |
| 20 | 30 | 70 | 7 |
| 25 | 30 | 70 | 7 |

5. “SF—HCOOH ACN Grad7 30 mm” Method
Column: Sunfire™ C8: 4.6 mm×150 mm, 3.5 μm (Waters ref. 186002732)
Eluent A: Water (HCOOH-0.02%); Eluent B=CH₃CN) with elution gradient
Elution condition: gradient

| | HCO₂H at | | |
|---|---|---|---|
| Min. | 0.2‰ | MeCN | Curve |
| | 50 | 50 | |
| 9 | 5 | 95 | 7 |
| 22 | 5 | 95 | 7 |
| 27 | 50 | 50 | 6 |
| 30 | 50 | 50 | 6 |

6. “SF—HCOOH ACN Grad12 45 mm” Method
Column: Sunfire™ C8: 4.6 mm×150 mm, 3.5 μm (Waters ref. 186002732)
Eluent A: Water (HCOOH-0.02%); Eluent B=CH₃CN) with elution gradient
Elution condition: gradient

| | HCO₂H at | | |
|---|---|---|---|
| Min. | 0.2‰ | MeCN | Curve |
| | 10 | 90 | |
| 5 | 0 | 100 | 6 |
| 35 | 0 | 100 | 6 |
| 40 | 10 | 90 | 6 |
| 45 | 10 | 90 | 6 |

### I—Obtaining the Starting Cyclic Units, Compounds of Formula VII_{A}, Shown Below

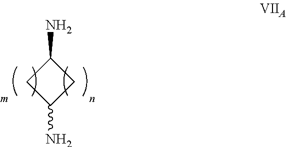

### I-1: Derivatives of Cyclopropane

### I-1-a: Cyclopropanes in Cis Relative Configuration

The cyclopropane rings, n=0 and m=1, of cis configuration are obtained from the anhydride described in the literature, commercial anhydride. The starting products are described in the various publications cited in the following list:
(1) Skatteboel L.; Stenstroem Y. Acta Chemica Scandinavica 1989, 43, 1, 93-96
(2) Csuk, R.; von Scholz, Y. Tetrahedron, 1994, 50, 35, 10431-10442
(3) Payne, G B. J. Org. Chem. 1967, 32, 3351-3355
(4) Kennewell P. D., Matharu S., Taylor J. B., Westwood R., Sammas P. G. J. of the Chemical Society, Perkin Transactions 1: Organic and Bioorganic Chemistry, 1982, 2553-2562
(5) Majchrzak M. W., Kotelko A., Lambert J. B. Synthesis, 1983, 6, 467-470
(6) Mohr P., Waespe-Sarcevic N., Tamm C., Gawronska K., Gawronsky J. K. Helvetica Chimica Acta, 1983, 66, 2501-2511
(7) Tufariello J. J., Milowsky A. S., Al-Nuri M., Goldstein S. Tet. Lett., 1987, 28, 267-270.
   The corresponding cis diamines are obtained by the method described in the publication of reference (8) by carrying out a reaction of the Curtius type.
(8) Reddy V. K., Valasinas A., Sarkar A., Basu H. S., Marton L. J., Frydman B. J. of Med. Chem., 1998, 41, 4723-4732.
   The various steps are shown in the following reaction diagram:

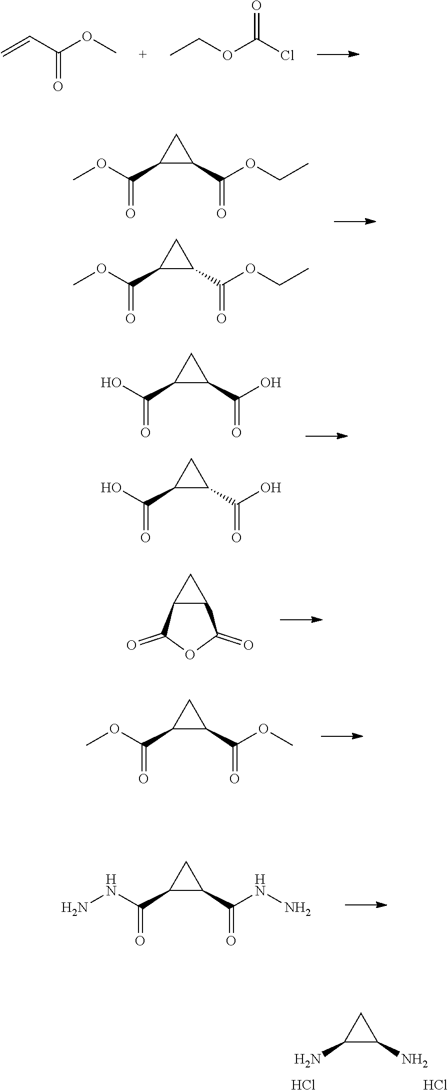

### I-1-b: Cyclopropanes in Trans Relative Configuration

The trans diacid is commercially available from Aldrich. If they are prepared, the cyclopropanes of trans relative configuration can be obtained on the basis of condensation of the chloroacetate with an acrylic derivative.
The products used were synthesized on the basis of the procedures described in references (1) to (8) cited above, reference (8) relating in particular to the Curtius reaction for obtaining the trans diamine.
The various steps are shown in the following reaction diagram:

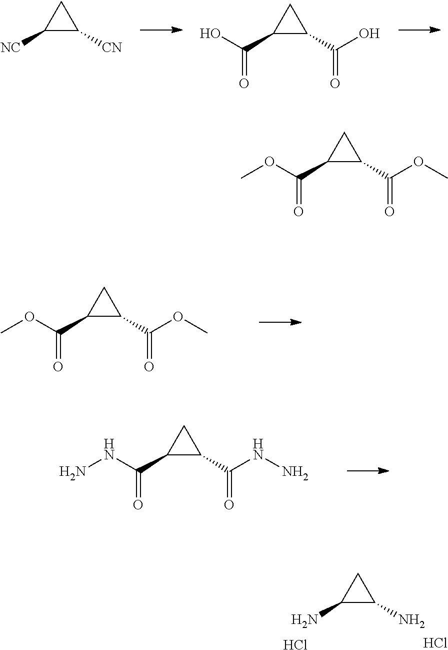

### I-2: Derivatives of Cyclobutanes

They are prepared by the same methods as those described above.

### I-3: Derivatives of Cyclopentane Diamine

### I-3-a: Cis and Trans Relative Configurations, in Position −1,2

They are obtained in three steps starting from the corresponding cyclopentanediol. This sequence is used in the two series, cis and trans. The stereochemistry of the functional carbon is not altered by the later reactions providing the diamine.
The cis and trans sequential syntheses are described in the following references:
(9) Kuppert D., Sander J., Roth C., Woerle M., Weyhermueller T., Reiss G J., Schilde U., Mueller I., Hegetschweiler K. European Journal of inorganic chemistry, 2001, 10, 2525-2542.
(10) Gouin S. G, Gestin J. F., Joly K., Loussouarn A., Reliquet A., Meslin J. C., Deniaud D. Tetrahedron, 2002, 16, 1131-1136.
(11) Goeksu S., Secen H., Suetbeyaz Y. Synthesis, 2002, 16, 2373-2378.
   For example, for the cis compound, the three-step synthesis is shown below:

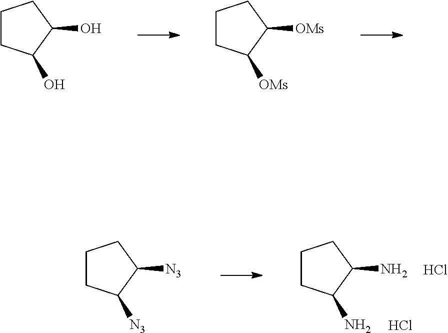 [SMILES: O[C@@H]1CCC[C@@H]1O ; CS(=O)(=O)O[C@H]1CCC[C@H]1OS(C)(=O)=O ; [N-]=[N+]=N[C@H]1CCC[C@H]1N=[N+]=[N-] ; N[C@@H]1CCC[C@@H]1N ; Cl ; Cl]

### I-3-b: Cis and Trans Relative Configurations, in Position −1,3

The 1,3-diaminocyclopentanes have been described since 1925, in particular by Diels and in Pfizer, AstraZeneca and Roche patents. The procedures used can be found in these patents and publications, the references of which are given below:
(12) Diels, Blom, Koll Justus Liebigs Annalen der Chemie I, 1925, 443, 247.
(13) Cohen S. G, Journal of American Chemical Society, 1961, 83, 2895-2900.
(14) Minisci F., Gazzetta Chimica Italia, 1964, 94, 67-90.
(15) Blanchard, Comptes Rendus des Séances de l'Académie des Sciences, Série Sciences Chimiques, 1970, 270, 657.
(16) AstraZeneca AB, AstraZeneca UK Limited, Patent WO2007/138277 A1, 2007.
(17) Hoffmann-La Roche A G, Patent WO2008/650210A1 2008.
(18) Pfizer Products Inc., Patent WO2008/65500 A2, 2008.

### II—Obtaining the Precursors of Side Chains, Compounds of General Formula VIII

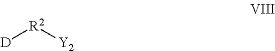

### II-1: Saturated Fatty Acids, Compounds of Formula VIII_{A}

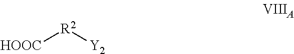

The fatty acids used, corresponding to the above formula with R²—Y₂ an alkyl chain comprising from 7 to 29 carbon atoms, saturated or unsaturated having a variable number of double bonds, are commercially available: for example, oleic acid, myristic acid, and palmitic acid will be used.

### II-2: α,β-unsaturated fatty acids, compounds of Formula VIII_{A}

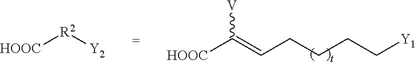

These derivatives correspond to the above formula with:
V representing a hydrogen, fluorine, chlorine, bromine, or iodine atom or single alkyl group,
t varying from 1 to 25,
Y₁ and R² having the meanings stated above.
   The α,β-unsaturated fatty acids are obtained by partial reduction of protected or unprotected lactone or lactam, followed by a reaction of the Wittig-Horner type and a saponification. Their synthesis is described in patent FR2911338.
   The 3 steps of the procedure are described in the following examples:

### Example 1

### Step a, Partial Reduction of the Lactone to Lactol

30 g of lactone is dissolved in 10 volumes of toluene, under a nitrogen atmosphere. The mixture is cooled down to −78° C. and 1.01 equivalents of Dibal-H (ACROS) in solution at 20% in toluene are added dropwise, keeping the temperature at −78° C. The mixture is stirred for 2 hours at −78° C. Eight volumes of a saturated solution of Rozen salts (double tartrate salts; ACROS) are added at −78° C. After 18 hours of vigorous stirring at ambient temperature, the two-phase mixture is filtered on Celite, and then extracted with ethyl acetate. The organic phases are washed with a saturated NaCl solution, dried over MgSO₄, filtered and concentrated under vacuum to give a crude product weighing 30 g (containing some traces of diol). The lactol, in open form-cyclic form equilibrium, is used as it is, without additional purification.

### Example 2

the following 8-hydroxyoctanal is prepared according to Example 1; its analytical characteristics are given below:

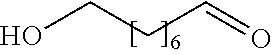

Characterization, step a, open form:

TLC: Rf=0.4 (heptane/ethyl acetate 6/4)

¹H NMR (300 MHz, CDCl₃): δ 1.34-1.68 (m, 10H); 2.45 (t, J=5.4 Hz, 2H); 3.66 (t, J=6.6 Hz, 2H); 9.78 (t, J=1.8 Hz, 1H).

### Example 3

### Step b, Wittig-Horner Reaction

19 g of lactol obtained in step a is diluted in 13 volumes of ethanol. 1.2 equivalents of triethylphosphonoacetate are added to the mixture in the presence of 1.5 equivalents of potassium carbonate. The reaction medium is heated at 40° C. for 18 hours. At ambient temperature, the mixture is hydrolysed with 10 volumes of distilled water and extracted with ethyl acetate. The organic phases are washed with a saturated NaCl solution, dried over MgSO₄, filtered and concentrated under vacuum to give a crude product weighing 20 g.
The ester obtained is purified by chromatography with the eluent mixture heptane/ethyl acetate 7/3. 15 g of product is obtained (53% yield).

### Example 4

the following compound is prepared according to Example 3; its analytical characteristics are as follows:

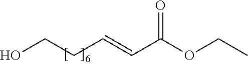

Characterization, Step b, with V Equal to Hydrogen:

TLC: Rf=0.4 (heptane/ethyl acetate 7/3)

¹H NMR (300 MHz, CDCl₃): δ 1.24-1.38 (m, 9H); 1.43-1.50 (m, 2H); 1.51-1.57 (m, 2H); 2.15-2.21 (q, 2H); 3.60-3.64 (t, 2H); 4.14-4.20 (t, 2H); 5.77-5.82 (d, J=15.6 Hz, 1H); 6.91-6.98 (dt, J=15.6 Hz, 1H).

### Example 5

This step is also carried out in a fluorinated series starting from triethyl 2-fluoro-2-phosphonoacetate. Two isomers are then possible: E and/or Z. The equivalent of the above molecule in a fluorinated series is prepared according to Example 3 and is shown below:

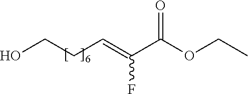

Characterization, Step b, with V Equal to Fluorine:

TLC: Rf=0.43 (heptane/ethyl acetate 7/3)

¹H NMR (300 MHz, CDCl₃): δ 1.28 (t, 6H); 1.30-1.65 (m, 20H); 2.16 (q, 4H); 2.27 (m, 2H); 2.52 (m, 2H); 3.66-3.71 (t, 4H); 5.99-6.11 (dt, J=21.0 Hz configuration E, 1H); 6.18-6.34 (dt, J=33.0 Hz configuration Z, 1H).

### Example 6

### Step c, Saponification Reaction

0.60 g of hydroxy ester obtained in the preceding step b is dissolved in 10 volumes of tetrahydrofuran. 2.4 equivalents of a 2M soda solution are added slowly. The mixture is heated at 65° C. for 3 hours. Once the reaction has ended, the mixture is hydrolysed by adding a 3M hydrochloric acid solution, until pH=2 is obtained. The mixture is concentrated to dryness and the aqueous phase is then extracted with ethyl acetate. The organic phases are washed with a saturated NaCl solution, dried over MgSO₄, filtered and concentrated under vacuum to give a crude product weighing 0.6 g.
The unsaturated hydroxy acid VIII_{A} is obtained, in the form of a white solid, by recrystallization from cold acetonitrile, m=0.37 g (yield equal to 71%).

### Example 7

it is prepared by the protocol described in Example 6. In the case of the following compound, the analytical characteristics are as follows:

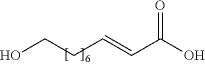

Characterization, step c:

TLC: Rf=0.1 (heptane/ethyl acetate 6/4)

¹H NMR (300 MHz, CDCl₃): δ 1.33-1.37 (m, 6H); 1.45-1.49 (m, 2H); 1.55-1.58 (m, 2H); 2.20-2.25 (q, 2H); 3.62-3.66 (t, 2H); 5.79-5.84 (d, J=15.6 Hz, 1H); 7.03-7.10 (dt, J=15.6 Hz, 1H)

Mass spectroscopy: [M±Na]⁺ 209 (calculated 186)

Melting point: 62.5° C.±1° C.

### Example 8

This step is also carried out in a fluorinated series. The molecule shown below is prepared according to Example 6, in a fluorinated series, and is characterized by:

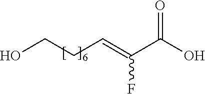

### Characterization:

TLC: Rf=0.12 (heptane/ethyl acetate 6/4)

¹H NMR (300 MHz, CDCl₃): δ 1.30-1.65 (m, 20H); 2.27 (m, 2H); 2.52 (m, 2H); 3.66-3.71 (t, 4H); 5.99-6.11 (dt, J=21.0 Hz configuration E, 1H); 6.18-6.34 (dt, J=33.0 Hz configuration Z, 1H).

### III—Access to the Pseudo-Ceramides, Symmetrical Compounds, Formula II_{A}

The compounds of Formula II_{A}

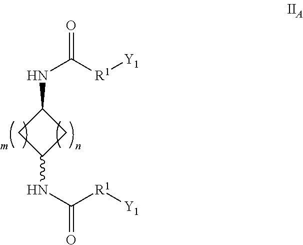

are obtained by one of the two methods described below:

method A=amide formation;

method B=amide formation followed by a Wittig-Horner reaction.

### III-1—Method A: Amide Formation

This method comprises an amide formation reaction optionally followed by a step of deprotection of Y₁. The acylation reaction is represented by the following equation:

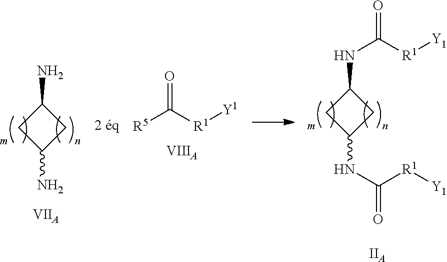

### III-1-1 Amide Formation

### Example 9

### Access to the Symmetrical Compounds by Amide Formation

1 equivalent of carboxylic acid VIII_{A} is dissolved in 10 volumes of tetrahydrofuran, under inert atmosphere. The diamine cyclic unit in trans series or cis series is added (0.5 equivalents), as well as 2.5 equivalents of 1-(3-dimethylaminopropyl)-3-ethyl-carbodiimide hydrochloride and 1.2 equivalents of 1-hydroxybenzotriazole. The suspension is cooled down to 0° C. and 3 equivalents of N,N-diisopropylethylamine are added slowly. The addition of a few drops of N,N′-dimethylformamide allows complete solubilization. The reaction medium is stirred for 16 h at ambient temperature. Analysis by thin-layer chromatography is used to check for the end of the reaction. The mixture is concentrated under vacuum. The residue is taken up in dichloromethane and distilled water. It is extracted with dichloromethane three times. The combined organic phases are washed with a 2M HCl solution and then with a saturated NaCl solution. They are dried over Na₂SO₄, filtered and concentrated, giving a brown oil, which is purified by silica gel chromatography (eluent dichloromethane/methanol 98/2). A pure product is obtained with a yield between 50% and 95%.

### Example 10

The compounds 1 to 161 are prepared according to the protocol of Example 9. The analytical characteristics of compound 27 are given below:

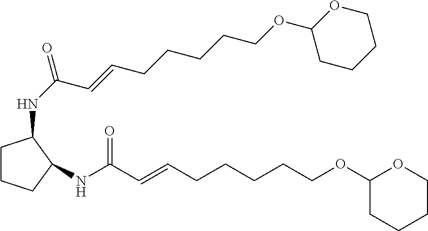 [SMILES: O=C(/C=C/CCCCCOC1CCCCO1)N[C@H]1CCC[C@H]1NC(=O)/C=C/CCCCCOC1CCCCO1]

Characterization: compound 27

TLC: Rf=0.3 (dichloromethane/methanol 98/2)

¹H NMR (300 MHz, CDCl₃): δ 1.30-1.78 (m, 28H); 2.10 (m, 6H); 3.30 (m, 2H); 3.45 (m, 2H); 3.65 (m, 2H); 3.80 (m, 2H); 4.15 (m, 2H); 4.50 (m, 2H); 5.67-5.73 (d, J=15.3 Hz, 2H); 6.18 (d, 2H); 6.70-6.776 (dt, J=15.3 Hz, 2H)

Mass spectroscopy: [M+Na]⁺571.3 (calculated 548.77)

HPLC: method HCOOH_ACN_gradient 1, tR=7.2 min, 95% at 210 nm.

### III-1-2 Deprotections

### Example 11

### Deprotection of the Terminal Alcohol Function: Hydrolysis of a Tetrahydropyran Unit

In certain cases, the acid derivative used bears a protective group in the form of —OTHP on the terminal alcohol function. The diprotected diamide compound is dissolved in 50 volumes of methanol. A catalytic quantity of p-toluenesulphonic acid is added and the mixture is stirred at 40° C. for 4 h. Monitoring by thin-layer chromatography provides a check for the end of the reaction. The mixture is then concentrated under vacuum; the residue is taken up in dichloromethane and distilled water. After several extractions with dichloromethane, the organic phases are washed with a saturated NaCl solution. They are dried over MgSO₄, filtered and concentrated under vacuum. The residue is purified by trituration in a water/ethyl acetate mixture or on a silica column, giving a fraction of pure product with yields close to 70%.

### Example 12

### Deprotection of the Terminal Alcohol Function: Hydrolysis of a Tert-Butyldiphenylsilyl Unit

In certain cases, the acid derivative used in the peptide coupling bears a protective group in the “tBdPhSiO—” form on the terminal alcohol function. The diprotected diamide compound is dissolved in 15 volumes of tetrahydrofuran. At 0° C., a solution of tetrabutylammonium fluoride (3 equivalents at 1M in THF) is added slowly. After stirring for 3 h at ambient temperature, monitoring by TLC provides a check for the end of the reaction. The reaction medium is then hydrolysed by adding a saturated NH4Cl solution. The mixture is extracted three times with ethyl acetate and the combined organic phases are washed with a saturated NaCl solution. After drying over MgSO₄ and filtration, the organic solvent is removed under vacuum. The residue obtained is triturated in organic mixtures or is purified by silica gel chromatography.

### Example 13

### Deprotection of the Terminal Alcohol Function: Hydrolysis of an Acetate Unit

In certain cases, the acid derivative used in the amide formation reaction bears a protective group in the —OAc form on the terminal alcohol function. The diprotected diamide compound is dissolved in 4 volumes of methanol. At ambient temperature, a freshly prepared aqueous solution of potassium carbonate (0.9 equivalent) and of potassium hydrogen carbonate (1.7 equivalents) is added. The reaction medium is stirred for 4 hours. Monitoring by TLC provides a check for complete deprotection. The mixture is then concentrated to dryness and then taken up in ethyl acetate and water. After trituration, the solid is filtered and dried under vacuum. The yield varies between 30% and 70%, depending on the length of the chain.

### Example 14

the deprotected product compound 36 is obtained by the protocol described in Example 13. Its analytical characteristics are as follows:

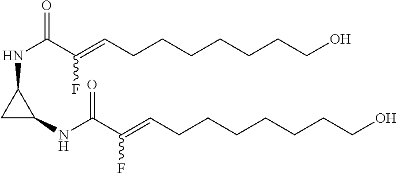 [SMILES: O=C(N[C@H]1C[C@H]1NC(=O)C(F)=CCCCCCCCO)C(F)=CCCCCCCCO]

Characterization: compound 36

TLC: Rf=0.15 (dichloromethane/methanol 98/2)

¹H NMR (300 MHz, CDCl₃): δ 0.89 (m, 2H); 1.27-1.60 (m, 16H); 2.20 (m, 4H); 2.64 (m, 4H); 2.90 (m, 4H); 3.65 (t, 2H); 5.70-5.84 (dt, J=24.6 Hz, 2H); 6.21-6.04 (dt, J=37.8 Hz, 2H)

Mass spectroscopy: [M+Na]⁺ 467.2 (calculated 444.57)

HPLC: method HCOOH_ACN_gradient 1, tR=10.6 min, 97% at 240 nm

### Example 15

### Deprotection of the Terminal Amine Function: Hydrolysis of a Carbamate Unit

In certain cases, the acid derivative used in the amide formation reaction bears a protective group in the —NHBoc form on the terminal amine function. The diprotected diamide compound is dissolved in 2 volumes of diethyl ether. A solution of dry hydrochloric acid in diethyl ether (2M) is added and the reaction medium is stirred at ambient temperature for 2 h. Monitoring by TLC provides a check for the end of the reaction. After concentrating to dryness, the residue is triturated in dichloromethane, giving a dihydrochloride salt of the desired compound, with a yield between 70% and 95%.

### Example 16

the deprotected product, compound 101, is obtained by the protocol described in Example 15. Its analytical characteristics are as follows:

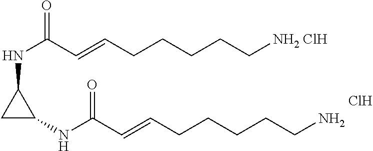

Characterization: compound 101

¹H NMR (300 MHz, DMSO-d₆): δ 0.89 (m, 2H); 1.30-1.57 (m, 12H); 2.12 (m, 4H); 2.51-2.76 (m, 8H); 5.62-5.82 (dt, J=15.0 Hz, 2H); 6.55-6.63 (dt, J=30.0 Hz, 2H); 7.93 (s, 4H); 8.15 (s, 2H).

### III-2—Method B, Coupling and Wittig-Horner Reaction, Use of a Phosphonoacetamide Intermediate

### III-2-1: Formation of a Diphosphonoacetamide Intermediate

### Example 17

### Preparation of the Phosphonoacetamides

Diethylphosphonoacetic acid (4 equivalents) is diluted in dichloromethane (14 volumes) under inert atmosphere. The reagent O-(7-azabenzotriazol-1-yl)-N,N,N′,N′-tetramethyluronium hexafluorophosphate (4.4 equivalents) as well as triethylamine (6.5 equivalents) are added. After stirring for five minutes at ambient temperature, the diamine cyclic unit (1 equivalent) is added and the reaction medium is heated at 50° C. for 30 min. Monitoring by thin-layer chromatography provides a check for the end of the reaction. The mixture is then hydrolysed by adding distilled water and then by adding a saturated NH4Cl solution. After two extractions with ethyl acetate, the organic phases are washed with a saturated NaCl solution, dried over MgSO₄, filtered and concentrated under vacuum. The residue is purified on a silica column with a dichloromethane/methanol gradient. The pure product is obtained with a yield above 95%.

### Example 18

Compound 144 is obtained using the protocol of Example 17. The analytical characteristics of compound 144 are as follows:

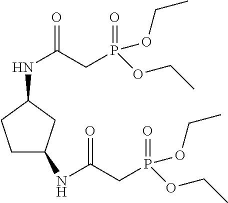 [SMILES: CCOP(=O)(CC(=O)N[C@@H]1CC[C@H](NC(=O)CP(=O)(OCC)OCC)C1)OCC]

Characterization: compound 144, diphosphonoacetamide intermediate:

TLC: Rf=0.2 (dichloromethane/methanol 95/5)

¹H NMR (300 MHz, DMSO-d₆): δ 1.12 (m, 2H); 1.22 (t, 12H); 1.42 (m, 2H); 1.83 (m, 2H); 2.72 (d, 4H); 3.42 (m, 2H); 4.0 (q, 8H); 8.04 (d, 2H)

Mass spectroscopy: [M+H]⁺=457.2; [M−H]⁻=455.2 (calculated 456.42)

HPLC: method HCOOH_ACN_gradient 1, tR=7.28 min, 94% at 210 nm.

### III-2-2: Reaction of the Wittig-Horner Type of the Diphosphonoacetamide Derivative

### Example 19

### Access to the Symmetrical Compounds by Reaction of the Wittig-Horner Type on the Phosphonoacetamide

The diphosphonoacetamide compound is used in a reaction of the Wittig-Horner type: under an inert atmosphere, 1 equivalent of diphosphonoacetamide product is dissolved in tetrahydrofuran (10 volumes). A base of the K₂CO₃ type (4 equivalents) and the aldehyde derivative (4 equivalents) are added to the reaction medium. The latter is heated at 50° C. overnight with stirring. Monitoring by TLC provides a check for the end of the reaction. At ambient temperature, the mixture is hydrolysed by adding distilled water. After three extractions with ethyl acetate, the organic phases are washed with a saturated NaCl solution, dried over MgSO₄, filtered and concentrated under vacuum. The crude residue is purified by trituration in dichloromethane: the insoluble salts are removed by filtration whereas the filtrate is concentrated, giving the desired product.

### III-2-3: Formation of a Fluorinated Diphosphonoacetamide Intermediate

### Example 20

### Preparation of Fluorinated Phosphonoacetamide

Diethylphosphonofluoroacetic acid (3 equivalents) is diluted in dichloromethane (14 volumes) under inert atmosphere. The reagent O-(7-azabenzotriazol-1-yl)-N,N,N′,N′-tetramethyluronium hexafluorophosphate (3 equivalents) as well as triethylamine (6.4 equivalents) are added. After stirring for five minutes at ambient temperature, the diamine cyclic unit (1 equivalent) is added and the reaction medium is heated at 50° C. for 30 min. Monitoring by thin-layer chromatography provides a check for the end of the reaction. The mixture is then hydrolysed by adding distilled water and then by adding a saturated NH4Cl solution. After two extractions with ethyl acetate, the organic phases are washed with a saturated NaCl solution, dried over MgSO₄, filtered and concentrated under vacuum. The residue is purified on a silica column with a dichloromethane/methanol gradient. The pure product is obtained with a yield above 90%.

### Example 21

Compound 145 is obtained using the protocol of Example 20. The analytical characteristics of compound 145 are as follows:

Characterization of the fluorinated diphosphonoacetamide intermediate: compound 145

TLC: Rf=0.3 (dichloromethane/methanol 85/15)

¹H NMR (300 MHz, DMSO-d₆): δ 5.21-5.50 (m, 2H); 4.25 (m, 8H); 3.25 (m, 2H), 1.80-2.14 (m, 2H), 1.40 (t, 12H), 1.38 (m, 4H).

Mass spectroscopy: [M+H]⁺=493.1; [M−H]⁻=491.1 (calculated 492.4)

HPLC: method HCOOH_ACN_gradient 1, tR=8.18 min

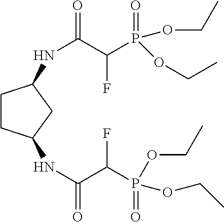 [SMILES: CCOP(=O)(OCC)C(F)C(=O)N[C@@H]1CC[C@H](NC(=O)C(F)P(=O)(OCC)OCC)C1]

### III-2-4: Reaction of the Fluorinated Diphosphonoacetamide Derivative in a Reaction of the Wittig-Horner Type

### Example 22

### Preparation of the Symmetrical Compounds by Reaction of the Wittig-Horner Type on the Fluorinated Diphosphonoacetamide

The diphosphonoacetamide compound thus obtained is used in a reaction of the Wittig-Horner type: under an inert atmosphere, 1 equivalent of diphosphonoacetamide product is dissolved in tetrahydrofuran (10 volumes). A base of the K₂CO₃ type (4 equivalents) and the aldehyde derivative (4 equivalents) are added to the reaction medium. The latter is heated at 50° C. overnight with stirring. Monitoring by TLC provides a check for the end of the reaction. At ambient temperature, the mixture is hydrolysed by adding distilled water. After three extractions with ethyl acetate, the organic phases are washed with a saturated NaCl solution, dried over MgSO₄, filtered and concentrated under vacuum. The crude residue is purified by trituration in dichloromethane: the insoluble salts are removed by filtration whereas the filtrate is concentrated, giving the desired product (description in Table 2).
The analytical description of the phosphonoacetamides XVIII obtained corresponding to the following diagram is given in Table 1 below. The reference of the compound is indicated by “c” followed by its number.

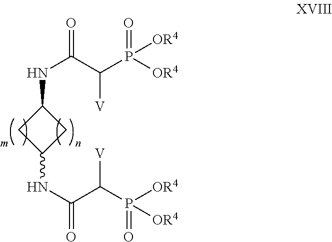

TABLE 1

| Compound | | | config- | | ¹H | | | |
|---|---|---|---|---|---|---|---|---|
| No. | n | m | uration | V | NMR ^{a} | LC | MS | appearance |
| c144 | 1 | 2 | cis | H | OK | 7.28 min¹ | [M + H]⁺ 457.2 | Colourless oil |
| c145 | 1 | 2 | Cis | F | OK | 8.18 min¹ | [M + H]⁺ 493.1; | Colourless oil |
| | | | | | | | [M − H]⁻ 491.1 | |

^{a} OK = coherent spectrum
¹X-Terra column, gradient 1

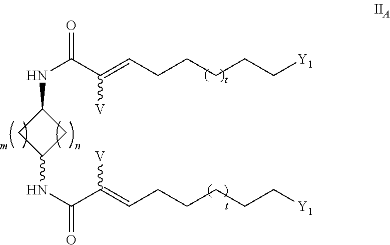

The analytical characteristics of the symmetrical compounds of Formula II_{A} shown above, derived from α,β-unsaturated fatty acids described previously, are summarized in Table 2 below:

TABLE 2

| Compound | | | Config- | | | | | LC and | | | |
|---|---|---|---|---|---|---|---|---|---|---|---|
| No. | n | m | uration | V | t | Y₁ | ¹H NMR^{a} | melting point | | MS | appearance |
| c1 | 0 | 1 | cis | H | 1 | OH | OK | 6.93 | min¹ | [M + H]⁺ 353.1 | Beige solid |
| c2 | 0 | 1 | cis | H | 2 | OH | OK | 7.84 | min¹ | [M + H]⁺ 380.9 | White solid |
| | | | | | | | | | | [M − H]⁻ 379.0 | |
| c3 | 0 | 1 | cis | H | 3 | OH | OK | 9.08 | min¹ | [M + Na]⁺ 431.3 | White solid |
| | | | | | | | | | | [M − H]⁻ 407.3 | |
| c4 | 0 | 1 | cis | H | 4 | OH | OK | 10.34 | min¹ | [M + H]⁺ 437.1 | White solid |
| | | | | | | | | | | [M − H]⁻ 435.2 | |
| c5 | 0 | 1 | cis | H | 5 | OH | OK | 11.16 | min¹ | [M + H]⁺ 465.4 | White solid |
| | | | | | | | | | | [M − H]⁻ 463.3 | |
| c6 | 0 | 1 | cis | H | 6 | OH | OK | 11.87 | min¹ | [M + Na]⁺ 515.4 | White solid |
| | | | | | | | | | | [M − H]⁻ 491.3 | |
| c7 | 0 | 1 | cis | H | 7 | OH | OK | NA | | [M + Na]⁺ 543.3 | White solid |
| | | | | | | | | | | [M − H]⁻ 519.4 | |
| c8 | 1 | 2 | cis | F | 1 | OTHP | OK | 10.0 | min⁷ | [M − H]⁻ 583.3 | Colourless oil |
| | | | | Z/E | | | | | | | |
| c9 | 0 | 1 | cis | H | 9 | OH | NA | 10.09 | min⁷ | [M + H]⁺ 577.3 | White solid |
| | | | | | | | | | | [M − H]⁻ 575.4 | |
| c10 | 0 | 1 | cis | H | 10 | OH | NA | 11.22 | min⁷ | [M + H]⁺ 605.4 | White solid |
| | | | | | | | | | | [M − H]⁻ 603.6 | |
| c11 | 0 | 1 | cis | H | 11 | OH | NA | 12.35 | min⁷ | [M + Na]⁺ 655.4 | White solid |
| c12 | 0 | 3 | cis | H | 1 | OH | OK | 7.58 | min¹ | [M + H]⁺ 381.2 | Beige solid |
| c13 | 0 | 3 | cis | H | 2 | OH | OK | 8.57 | min¹ | [M + H]⁺ 409.2 | White solid |
| | | | | | | | | | | [M − H]⁻ 407.2 | |
| c14 | 0 | 3 | cis | H | 3 | OH | OK | 9.89 | min¹ | [M + H]⁺ 437.2 | Colourless liquid |
| | | | | | | | | | | [M − H]⁻ 435.2 | |
| c15 | 0 | 3 | cis | H | 4 | OH | OK | 10.82 | min¹ | [M + H]⁺ 465.2 | White solid |
| | | | | | | | | | | [M + HCOOH—H]⁻ | |
| | | | | | | | | | | 509.2 | |
| c16 | 0 | 3 | cis | H | 6 | OH | OK | 12.21 | min¹ | [M + H]⁺ 521.3 | Pale yellow solid |
| | | | | | | | | | | [M + HCOOH—H]⁻ | |
| | | | | | | | | | | 565.3 | |
| c17 | 0 | 3 | cis | H | 7 | OH | OK | 12.75 | min¹ | [M + H]⁺ 549.3 | White solid |
| c18 | 0 | 3 | cis | H | 10 | OH | OK | 11.74 | min⁷ | [M + Na]⁺ 655.3 | White solid |
| c19 | 0 | 3 | cis | H | 11 | OH | OK | 12.86 | min⁷ | [M + Na]⁺ 683.4 | White solid |
| c20 | 1 | 2 | cis | H | 1 | OH | OK | 7.49 | min¹ | [M + H]⁺ 381.2 | White solid |
| c21 | 1 | 2 | cis | H | 2 | OH | OK | 8.53 | min¹ | [M + H]⁺ 409.2 | White solid |
| | | | | | | | | | | [M + HCOOH—H]⁻ | |
| | | | | | | | | | | 453.1 | |
| c22 | 1 | 2 | cis | H | 3 | OH | OK | 9.70 | min¹ | [M + H]⁺ 437.2 | White solid |
| c23 | 1 | 2 | cis | H | 4 | OH | OK | 10.76 | min¹ | [M + H]⁺ 465.2 | White solid |
| c24 | 1 | 2 | cis | H | 7 | OH | OK | 12.75 | min¹ | [M + H]⁺ 549.3 | White solid |
| c25 | 1 | 2 | cis | H | 10 | OH | OK | 11.66 | min⁷ | [M + H]⁺ 655.3 | White solid |
| c26 | 1 | 2 | cis | H | 11 | OH | NA | 12.95 | min⁷ | [M + Na]⁺ 683.4 | White solid |
| c27 | 0 | 3 | cis | H | 1 | —OTHP | OK | 7.23 | min⁷ | [M + Na]⁺ 571.3 | White solid |
| c28 | 0 | 3 | cis | H | 2 | —OTHP | OK | 13.16 | min¹ | [M + Na]⁺ 599.4 | White solid |
| | | | | | | | | | | [M − H]⁻ 575.5 | |
| c29 | 1 | 2 | cis | H | 4 | —OTHP | OK | NA | | [M + H]⁺ 633.4 | White solid |
| c30 | 1 | 2 | cis | H | 7 | —OTHP | OK | 15.09 | min⁷ | [M + Na]⁺ 739.6 | White solid |
| c31 | 0 | 3 | cis | H | 7 | —OTHP | OK | 15.22 | min⁷ | [M + Na]⁺ 739.6 | White solid |
| c32 | 0 | 1 | cis | H | 10 | —OTHP | OK | 23.52 | min⁷ | [M + Na]⁺ 795.6 | White solid |
| c33 | 0 | 3 | cis | H | 11 | —OTHP | OK | NA | | [M + Na]⁺ 851.5 | White solid |
| c34 | 1 | 2 | cis | H | 11 | —OTHP | OK | 18.06 | min⁷ | NA | White solid |
| c35 | 0 | 1 | cis | F | 1 | OH | OK | 8.82 | min¹ | [M + Na]⁺ 410.9 | White solid |
| | | | | E/E | | | | | | [M − H]⁻ 387.0 | |
| c36 | 0 | 1 | cis | F | 3 | OH | OK | 10.65 | min¹ | [M + Na]⁺ 467.2 | White solid |
| | | | | | | | | | | [M − H]⁻ 443.3 | |
| c37 | 0 | 1 | cis | F | 4 | OH | OK | 11.55 | min¹ | [M + Na]⁺ 495.0 | White solid |
| | | | | | | | | | | [M − H]⁻ 471.1 | |
| c38 | 0 | 1 | cis | F | 5 | OH | OK | 12.30 | min¹ | [M + H]⁺ 501.0 | White solid |
| | | | | | | | | | | [M − H]⁻ 499.0 | |
| c39 | 0 | 1 | cis | F | 6 | OH | OK | 12.85 | min¹ | [M + H]⁺ 529.1 | Off-white solid |
| | | | | | | | | | | [M − H]⁻ 527.1 | |
| c40 | 0 | 1 | cis | F | 7 | OH | OK | 9.07 | min⁷ | [M + H]⁺ 579.2 | Beige solid |
| | | | | | | | | | | [M − H]⁻ 555.3 | |
| c41 | 0 | 1 | cis | F | 9 | OH | OK | 11.87 | min⁷ | [M + Na]⁺ 635.2 | White solid |
| c42 | 0 | 1 | cis | F | 10 | OH | OK | 12.42 | min⁷ | [M + Na]⁺ 663.4 | White solid |
| c43 | 0 | 1 | cis | F | 10 | OH | OK | 12.83 | min⁷ | [M + Na]⁺ 663.4 | White solid |
| | | | | E/E | | | | | | | |
| c44 | 0 | 1 | cis | F | 11 | OH | NA | 14.31 | min⁷ | [M + Na]⁺ 691.3 | White solid |
| | | | | | | | | | | [M − H]⁻ 668.1 | |
| c45 | 0 | 3 | cis | F | 3 | OH | OK | 11.13 | min¹ | [M + Na]⁺ 495.1 | White solid |
| | | | | Z/Z | | | | | | [M − H]⁻ 471.1 | |
| c46 | 0 | 3 | cis | F | 3 | OH | OK | 11.40 | min¹ | [M + Na]⁺ 495.1 | White solid |
| | | | | | | | | | | [M − H]⁻ 471.1 | |
| c47 | 0 | 3 | cis | F | 3 | OH | OK | 11.69 | min¹ | [M + Na]⁺ 495.1 | White solid |
| | | | | E/E | | | | | | [M − H]⁻ 471.1 | |
| c48 | 0 | 3 | cis | F | 4 | OH | NA | 12.10 | min¹ | [M + H]⁺ 501.2 | White solid |
| | | | | | | | | | | [M − H]⁻ 499.2 | |
| c49 | 0 | 3 | cis | F | 4 | OH | OK | 12.29 | min¹ | [M + H]⁺ 501.2 | White solid |
| | | | | E/E | | | | | | [M − H]⁻ 499.2 | |
| c50 | 0 | 3 | cis | F | 7 | OH | OK | 10.05 | min⁷ | [M + H]⁺ 585.2 | White solid |
| | | | | Z/Z | | | | | | [M − H]⁻ 583.2 | |
| c51 | 0 | 3 | cis | F | 7 | OH | OK | 10.73 | min⁷ | [M + H]⁺ 585.2 | White solid |
| | | | | E/E | | | | | | [M − H]⁻ 583.2 | |
| c52 | 0 | 3 | cis | F | 7 | OH | OK | 10.40 | min⁷ | [M + H]⁺ 585.2 | White solid |
| | | | | | | | | | | [M − H]⁻ 583.2 | |
| c53 | 0 | 3 | cis | F | 10 | OH | OK | 13.35 | min⁷ | [M + Na]⁺ 691.3 | White solid |
| c54 | 0 | 3 | cis | F | 11 | OH | OK | 14.81 | min⁷ | [M + Na]⁺ 719.4 | White solid |
| c55 | 1 | 2 | cis | F | 3 | OH | OK | 11.20 | min¹ | [M + Na]⁺ 495.2 | White solid |
| | | | | Z/Z | | | | | | [M − H]⁻ 471.3 | |
| c56 | 1 | 2 | cis | F | 3 | OH | OK | 11.44 | min¹ | [M + Na]⁺ 495.2 | White solid |
| | | | | | | | | | | [M − H]⁻ 471.3 | |
| c57 | 1 | 2 | cis | F | 3 | OH | OK | 11.70 | min¹ | [M + Na]⁺ 495.2 | White solid |
| | | | | E/E | | | | | | [M − H]⁻ = 471.3 | |
| c58 | 1 | 2 | cis | F | 4 | OH | OK | 11.86 | min¹ | [M + Na]⁺ 523.2 | Off-white solid |
| | | | | Z/Z | | | | | | [M − H]⁻ 499.2 | |
| c59 | 1 | 2 | cis | F | 4 | OH | OK | 12.08 | min¹ | [M + Na]⁺ 523.2 | White solid |
| | | | | | | | | | | [M − H]⁻ 499.2 | |
| c60 | 1 | 2 | cis | F | 7 | OH | OK | 10.40 | min⁷ | [M + H]⁺ 585.2 | White solid |
| | | | | | | | | | | [M − H]⁻ 583.1 | |
| c61 | 1 | 2 | cis | F | 10 | OH | OK | 13.52 | min⁷ | [M + Na]⁺ 691.3 | White solid |
| | | | | | | | | | | [M − H]⁻ 667.3 | |
| c62 | 1 | 2 | cis | F | 11 | OH | NA | 14.68 | min⁷ | [M + H]⁺ 697.3 | White solid |
| | | | | Z/Z | | | | | | [M − H]⁻ 695.4 | |
| c63 | 1 | 2 | cis | F | 11 | OH | NA | 15.03 | min⁷ | [M + H]⁺ 697.3 | White solid |
| | | | | | | | | 104-105° | C. | [M − H]⁻ 695.4 | |
| c64 | 1 | 2 | cis | F | 11 | OH | NA | 15.51 | min⁷ | [M + H]⁺ 697.3 | White solid |
| | | | | E/E | | | | | | [M − H]⁻ 695.4 | |
| c65 | 0 | 3 | cis | F | 3 | —OTHP | OK | NA | | [M + Na]⁺ 663.2 | Yellow solid |
| | | | | Z/Z | | | | | | [M − H]⁻ 639.3 | |
| c66 | 0 | 3 | cis | F | 3 | —OTHP | OK | NA | | [M + Na]⁺ 663.2 | Colourless liquid |
| | | | | | | | | | | [M − H]⁻ 639.3 | |
| c67 | 0 | 3 | cis | F | 3 | —OTHP | OK | NA | | [M + Na]⁺ 663.2 | Colourless liquid |
| | | | | E/E | | | | | | [M − H]⁻ 639.4 | |
| c68 | 1 | 2 | cis | F | 3 | —OTHP | OK | NA | | [M + Na]⁺ 663.2 | White solid |
| | | | | Z/Z | | | | 84-88° | C. | [M − H]⁻ 639.2 | |
| c69 | 1 | 2 | cis | F | 3 | —OTHP | OK | NA | | [M + Na]⁺ 663.2 | Colourless liquid |
| | | | | | | | | | | [M − H]⁻ 639.3 | |
| c70 | 1 | 2 | cis | F | 3 | —OTHP | OK | NA | | [M + Na]⁺ 663.2 | Colourless liquid |
| | | | | E/E | | | | | | [M − H]⁻ 639.2 | |
| c71 | 0 | 3 | cis | F | 4 | —OTHP | OK | 12.83 | min⁷ | [M + Na]⁺ 691.4 | Colourless liquid |
| c72 | 0 | 3 | cis | F | 4 | —OTHP | OK | 13.16 | min⁷ | [M + Na]⁺ 691.4 | Colourless liquid |
| | | | | E/E | | | | | | [M − H]⁻ 667.5 | |
| c73 | 1 | 2 | cis | F | 4 | —OTHP | OK | 12.54 | min⁷ | [M + Na]⁺ 691.5 | White solid |
| | | | | Z/Z | | | | | | [M − HCOOH—H]⁻ = | |
| | | | | | | | | | | 713.5 | |
| c74 | 1 | 2 | cis | F | 4 | —OTHP | OK | 12.79 | min⁷ | [M + Na]⁺ 691.5 | Colourless liquid |
| | | | | | | | | | | [M − HCOOH—H]⁻ | |
| | | | | | | | | | | 713.6 | |
| c75 | 0 | 3 | cis | F | 11 | —OTHP | OK | NA | | [M + Na]⁺ 887.5 | White solid |
| c76 | 1 | 2 | cis | F | 11 | —OTHP | OK | 20.52 | min⁷ | [M + Na]⁺ 887.5 | White solid |
| | | | | Z/Z | | | | | | [M − HCOOH—H]⁻ | |
| | | | | | | | | | | 909.5 | |
| c77 | 1 | 2 | cis | F | 11 | —OTHP | OK | 24.52 | min⁷ | [M + Na]⁺ 887.5 | White solid |
| c78 | 1 | 2 | cis | F | 11 | —OTHP | OK | NA | | NA | White solid |
| | | | | E/E | | | | | | | |
| c79 | 0 | 3 | cis | H | 7 | —H | OK | 17.29 | min⁷ | [M + H]⁺ 517.3 | White solid |
| c80 | 1 | 2 | cis | H | 7 | —H | OK | 19.17 | min¹ | [M + H]⁺ 517.3 | Beige solid |
| c81 | 0 | 3 | cis | F | 7 | —H | OK | 14.08 | min¹¹ | [M + H]⁺ 553.4 | White solid |
| | | | | Z/Z | | | | | | [M − H]⁻ 551.5 | |
| c82 | 0 | 3 | cis | F | 7 | —H | OK | 14.31 | min¹¹ | [M + H]⁺ 553.4 | White solid |
| c83 | 1 | 2 | cis | F | 7 | —H | OK | 14.24 | min¹¹ | [M + H]⁺ 553.4 | White solid |
| | | | | Z/Z | | | | | | | |
| c84 | 1 | 2 | cis | F | 7 | —H | OK | 14.39 | min¹¹ | [M + H]⁺ 575.3 | |
| c85 | 1 | 2 | cis | F | 7 | —H | OK | 14.79 | min¹¹ | [M + H]⁺ 553.4 | White solid |
| | | | | E/E | | | | | | [M − H]⁻ 551.6 | |
| c86 | 0 | 1 | cis | F | 7 | —H | OK | 20.30 | min⁷ | [M + Na]⁺ 547.2 | White solid |
| | | | | E/E | | | | | | [M − H]⁻ 523.2 | |
| c87 | 0 | 1 | cis | H | 3 | —OAc | OK | 11.99 | min¹ | [M + H]⁺ 493.3 | White solid |
| c88 | 0 | 1 | trans | H | 3 | —OAc | OK | 12.13 | min¹ | [M + H]⁺ 493.3 | Cream white solid |
| c89 | 0 | 1 | cis | F | 11 | —OAc | OK | NA | | [M + Na]⁺ 775.4 | White solid |
| c90 | 1 | 2 | cis | F | 7 | —OAc | OK | 12.26 | min⁷ | [M + Na]⁺ 663.3 | White solid |
| c91 | 0 | 1 | trans | F | 7 | OH | NA | 12.66 | min¹ | [M + H]⁺ 521.4 | Yellow solid |
| | | | | | | | | | | [M + Na]⁺ 543.4 | |
| c92 | 0 | 1 | Trans | F | 3 | OH | OK | 10.4 to 11.0 | min¹ | [M + H]⁺ 445.4 | White solid |
| | | | | | | | | | | [M − H]⁻ 443.3 | |
| c93 | 0 | 1 | trans | H | 10 | OH | OK | NA | | [M + Na]⁺ 627.5 | White solid |
| c94 | 0 | 1 | cis | F | 10 | OH | OK | NA | | [M + Na]⁺ 663.5 | White solid |
| | | | | E/E | | | | | | [M − H]⁻ 639.5 | |
| c95 | 0 | 1 | trans | H | 4 | OH | OK | 9.95 | min¹ | [M + H]⁺ 437.4 | White solid |
| c96 | 0 | 1 | trans | F | 11 | OH | OK | NA | | NA | White solid |
| | | | | E/E | | | | | | | |
| c97 | 0 | 1 | trans | H | 11 | OH | OK | NA | | [M + Na]⁺ 655.6 | White solid |
| c98 | 0 | 1 | cis | H | 8 | OH | OK | 8.60 | min⁷ | [M + H]⁺ 549.1 | White solid |
| | | | | | | | | | | [M − H]⁻ 547.2 | |
| c99 | 1 | 2 | cis | H | 1 | —OTHP | OK | NA | | [M + H]⁺ 549.1 | Beige solid |
| c100 | 0 | 3 | cis | F | 7 | H | OK | 14.60 | min¹¹ | [M + H]⁺ 553.3 | White solid |
| | | | | E/E | | | | | | [M − H]⁻ 551.4 | |
| c101 | 0 | 1 | trans | H | 1 | —NH₂ | OK | 2.5 | min¹ | [M + H]⁺ 351.4 | Yellow solid |
| c102 | 0 | 1 | trans | H | 1 | —NHBoc | OK | 11.96 | min¹ | [M + H]⁺ 551.4 | White solid |
| | | | | | | | | | | [M + HCOOH—H]⁻ | |
| | | | | | | | | | | 595.5 | |
| c103 | 0 | 1 | cis | F | 5 | —OtBdPh | OK | NA | | [M + Na]⁺ 1000.2 | Yellow paste |
| | | | | Z/Z | | | | | | [M − H]⁻ 976.2 | |
| c104 | 0 | 1 | cis | F | 5 | —OtBdPh | OK | NA | | [M + Na]⁺ 1000.1 | Yellow paste |
| | | | | | | | | | | [M − H]⁻ 976.2 | |
| c105 | 0 | 1 | cis | F | 5 | —OtBdPh | OK | NA | | [M + Na]⁺ 1000.1 | Yellow paste |
| | | | | E/E | | | | | | [M − H]⁻ 976.2 | |
| c106 | 1 | 2 | cis | F | 1 | —OTHP | OK | 10.46 | min⁷ | [M − H]⁻ 583.3 | Colourless oil |
| c107 | 0 | 3 | cis | F | 1 | —OTHP | OK | 9.80 | min⁷ | [M − H]⁻ 583.4 | White solid |
| c108 | 0 | 1 | cis | F | 1 | —OTHP | OK | 8.40 | min⁷ | [M + Na]⁺ 579.2 | White wax |
| | | | | | | | | | | [M − H]⁻ 555.3 | |
| c109 | 0 | 1 | cis | F | 1 | —OTHP | OK | NA | | NA | White solid |
| c110 | 0 | 1 | cis | F | 1 | —OTHP | OK | 9.06 | min⁷ | [M − H]⁻ = 555.1 | Colourless oil |
| c111 | 0 | 1 | cis | F | 1 | —OTHP | OK | 8.65 | min⁷ | [M − H]⁻ 555.1 | White wax |
| c112 | 0 | 1 | cis | F | 1 | OH | OK | 8.60 | min⁷ | [M + Na]⁺ 411.3 | Beige solid |
| c113 | 0 | 1 | cis | F | 1 | OH | OK | 8.32 | min¹ | [M + H]⁺ 389.3 | White solid |
| c114 | 1 | 2 | cis | F | 1 | OH | OK | NA | | [M + H]⁺ 417.2 | Golden oil |
| c115 | 1 | 2 | cis | F | 1 | OH | OK | NA | | [M + H]⁺ 417.2 | Golden oil |
| c116 | 1 | 2 | cis | F | 1 | OH | OK | NA | | [M + H]⁺ 417.2 | White wax |
| c117 | 0 | 3 | cis | F | 1 | OH | OK | 10.00 | min¹ | [M + H]⁺ 417.2 | Beige wax |
| c118 | 0 | 3 | cis | F | 1 | OH | OK | 9.29 | min¹ | [M + H]⁺ 417.3 | White solid |
| c149 | 1 | 2 | cis | F | 1 | OTHP | OK | 9.64 | min⁷ | [M − H]⁻ 583.3 | White wax |
| | | | | Z/Z | | | | | | | |

| | | | | | | | | | | |
|---|---|---|---|---|---|---|---|---|---|---|
| c150 | 0 | 3 | cis | F | 1 | OTHP | OK | NA | NA | Golden oil |
| | | | | E/E | | | | | | |
| c151 | 0 | 3 | cis | F | 1 | OTHP | OK | NA | NA | Golden oil |
| | | | | Z/E | | | | | | |
| c152 | 1 | 2 | cis | F | 1 | OTHP | OK | 9.67 + | [M − H]⁻ 583.3 | Golden oil |
| | | | | | | | | 10.06 + | | |
| | | | | | | | | 10.46 min⁷ | | |

^{a}OK = coherent spectrum
^{b}the superscript after the retention time refers to the method used.
¹Method HCOOH_ACN grad 1
⁷Method HCOOH_ACN grad 7
¹¹Method HCOOH_ACN grad 11

The analytical characteristics of the symmetrical compounds of Formula II_{A} shown below in which —R¹—Y₁ is an alkyl group, derived from saturated fatty acids, are presented in Table 3 below:

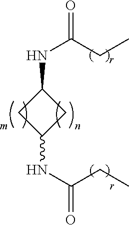

r has from 6 to 29 carbon atoms.

TABLE 3

| | | | Config- | | | | | |
|---|---|---|---|---|---|---|---|---|
| No. | n | m | uration | ^{a} | ¹H NMR^{b} | LC^{c} | MS | appearance |
| c119 | 0 | 1 | cis | Erucic acid | OK | 17.3 min¹² | [M + Na]⁺ 735.6 | White solid |
| | | | | (22:1(13)) | | | | |
| c120 | 0 | 1 | cis | Palmitoleic | OK | 15.8 min⁷ | [M + Na]⁺ 567.3 | White solid |
| | | | | acid (16:1(9)) | | | | |
| c121 | 0 | 1 | cis | Oleic acid | OK | 19.9 min⁷ | [M + Na]⁺ 623.5 | White solid |
| | | | | (18:1(9)) | | | | |
| c122 | 0 | 1 | cis | Linoleic acid | OK | 16.2 min⁷ | [M + Na]⁺ 619.4 | White solid |
| | | | | (18:2(9,12)) | | | | |
| c123 | 0 | 1 | cis | Linolenic acid | OK | 14.1 min⁷ | [M + Na]⁺ 615.4 | Beige solid |
| | | | | (10:3(9,12,15)) | | | | |
| c124 | 0 | 3 | cis | Erucic acid | OK | 19.5 min¹² | [M + H]⁺ 741.2 | White solid |
| | | | | (22:1(13)) | | | | |
| c125 | 0 | 3 | cis | Myristic acid | OK | 15.5 min⁷ | [M + Na]⁺ 543.3 | White solid |
| | | | | (14:0) | | | | |
| c126 | 0 | 3 | cis | Palmitoleic | OK | 16.2 min⁷ | [M + Na]⁺ 595.4 | Yellow oil |
| | | | | acid (16:1(9)) | | | | |
| c127 | 0 | 3 | cis | Oleic acid | OK | 20.5 min⁷ | [M + Na]⁺ 651.4 | White paste |
| | | | | (18:1(9)) | | | | |
| c128 | 0 | 3 | cis | Linoleic acid | OK | 16.7 min⁷ | [M + Na]⁺ 647.4 | Yellow oil |
| | | | | (18:2(9,12)) | | | | |
| c129 | 0 | 3 | cis | Linolenic acid | OK | 14.4 min⁷ | [M + Na]⁺ 642.7 | Yellow oil |
| | | | | (10:3(9,12,15)) | | | | |
| c130 | 1 | 2 | cis | Erucic acid | OK | 26.3 min⁹ | [M + H]⁺ 741.6 | White solid |
| | | | | (22:1(13)) | | | | |
| c131 | 1 | 2 | cis | Margaric acid | OK | 22.4 min⁷ | [M + Na]⁺ 627.6 | White solid |
| | | | | (17:0) | | | | |
| c132 | 1 | 2 | cis | Myristic acid | OK | 15.4 min⁷ | [M + Na]⁺ 543.3 | White solid |
| | | | | (14:0) | | | | |
| c133 | 1 | 2 | cis | Palmitoleic | OK | 15.9 min⁷ | [M + Na]⁺ 595.3 | White solid |
| | | | | acid (16:1(9)) | | | | |
| c134 | 1 | 2 | cis | Oleic acid | OK | 20.2 min⁷ | [M + Na]⁺ 651.5 | White solid |
| | | | | (18:1(9)) | | | | |
| c135 | 1 | 2 | cis | Linoleic acid | OK | 16.5 min⁷ | [M + Na]⁺ 647.4 | Yellow paste |
| | | | | (18:2(9,12)) | | | | |
| c136 | 1 | 2 | cis | Linolenic acid | OK | 14.3 min⁷ | [M + Na]⁺ 643.4 | Yellow paste |
| | | | | (10:3(9,12,15)) | | | | |
| c137 | 0 | 1 | cis | Myristic acid | OK | 15.2 min⁷ | [M + Na]⁺ 515.4 | White solid |
| | | | | (14:0) | | | | |

^{a} nomenclature of the fatty acids (t:u(v)): t = number of carbons, u = number of double bonds, v = the carbon or carbons bearing the double bonds).
^{b}OK = coherent spectrum; NA = not analysed
^{c}the superscript after the retention time refers to the method used.
¹²Method SF-HCOOH_ACN grad12
⁹Method HCOOH_ACN grad 9
⁷Method SF-HCOOH_ACN grad7

### IV—Access to the Asymmetrical Target Compounds, Cyclic Starting Product: Diamine

The synthesis comprises four steps: protection of one of the two amine functions, first amide formation carried out with the unprotected function by reaction with a fatty acid, deprotection of the blocked amine function and then second amide formation with a fatty acid different from that used in the first coupling.
The equations of the reaction diagram are shown below:

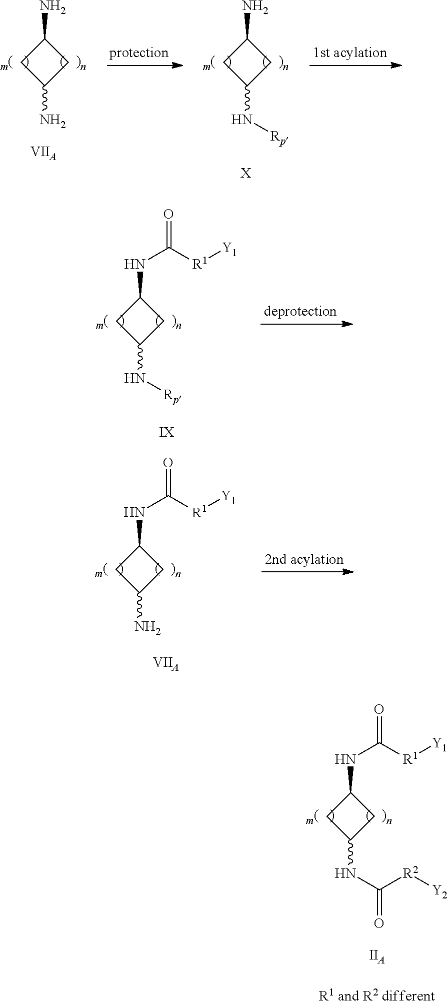

### IV-1: Monoprotection

### Example 23

### Preparation of the Compounds of Formula X by Protection of an Amine Function of the Compound of Formula VII_{A}

In this first step, the diamine compound VII_{A} is dissolved in tetrahydrofuran (10 volumes). Then at 0° C., 1.1 equivalent of Boc₂O and 1 equivalent of triethylamine are added. The reaction medium is stirred overnight at ambient temperature. Monitoring by TLC provides a check for the end of the reaction. The mixture is then concentrated under vacuum and then purified on a silica gel column, giving the mono-protected compound.

### IV-2: First Amide Formation

### Example 24

### Preparation of the Compounds of Formula IX

The mono-protected diamine compound X obtained in the preceding step (Example 23) is used in a coupling reaction in the presence of N,N-diisopropylethylamine (DIEA), 1-hydroxybenzotriazole (HOBT), 1-(3-dimethylaminopropyl)-3-ethyl-carbodiimide hydrochloride (EDCI), in order to obtain compound IX, according to the following protocol.
Compound X obtained previously (1 equivalent) is dissolved in 20 volumes of dichloromethane under an inert atmosphere. At ambient temperature, 1.1 equivalent of 1-hydroxybenzotriazole, 1.1 equivalent of 1-(3-dimethylaminopropyl)-3-ethyl-carbodiimide hydrochloride and 2 equivalents of N,N-diisopropylethylamine are added. After stirring for 5 minutes, compound VIII_{A} (1.2 equivalent) is added. The reaction medium is stirred for 18 h at ambient temperature. Monitoring by TLC provides a check for the end of the reaction. The mixture is then hydrolysed by adding water. After three extractions with ethyl acetate, the organic phases are washed with a saturated NaCl solution. They are then dried over MgSO₄, filtered and concentrated under vacuum. The crude residue is purified on a silica gel column eluted with a heptane/ethyl acetate gradient, giving a white solid with a yield close to 30%.

### IV-3: Deprotection

### Example 25

### Preparation of the Amidoamine VII_{D} by Deprotection of the Compound of Formula IX

The amine function protected by a Boc group is deprotected by the action of trifluoroacetic acid (2 equivalents) in solution in tetrahydrofuran (5 volumes), with stirring for 20 h. After concentrating to dryness, the compound VII_{D} obtained is used directly in the next step without additional purification.

### IV-4: Second Amide Formation

### Example 26

### Preparation of the Asymmetrical Target Compounds of Formula II_{B} by Amide Formation

The amidoamine VII_{D} obtained by Example 25 is used in peptide coupling with a carboxylic acid derivative different from that used for the first amide formation, in the presence of N,N-diisopropylethylamine (DIEA), 1-hydroxybenzotriazole (HOBT), 1-(3-dimethylaminopropyl)-3-ethyl-carbodiimide hydrochloride (EDCI) in dichloromethane, following the conditions of the protocol described in Example 24.
The asymmetrical compounds II_{B} are obtained.
The NMR characteristics of the examples prepared are shown in the following tables.
The shifts of the protons in positions 1, 2, 3 referenced on the diagram are given as well as the vinylic proton shifts, if present.
It should be noted that R^{1a} represents a linear or branched chain possessing from 1 to 30 carbon atoms, saturated or unsaturated, and in the case of an unsaturation, with the C═C double bond optionally substituted with a fluorine, chlorine, or bromine atom or with a —CF₃ group.

TABLE A

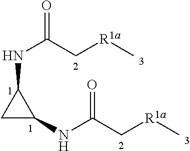

| | ¹H NMR description (300 MHz, CDCl₃, δ ppm) |
|---|---|
| c119 | 5.92 (s, 2H, NH); 5.38 (t, 4H, Hvinylic); 2.74 (m, 2H, H₁); 2.18 |
| | (t, 4H, H₂); 0.91 (t, 6H, H₃) |
| c120 | 5.92 (s, 2H, NH); 5.38 (m, 4H, Hvinylic); 2.74 (m, 2H, H₁); 2.19 |
| | (t, 4H, H₂); 0.91 (t, 6H, H₃) |
| c121 | 5.93 (s, 2H, NH); 5.37 (m, 4H, Hvinylic); 2.73 (m, 2H, H₁); 2.19 |
| | (t, 4H, H₂); 0.91 (t, 6H, H₃) |
| c122 | 5.96 (s, 2H, NH); 5.32 (m, 8H, Hvinylic); 2.80 (m, 2H, H₁); 2.18 |
| | (t, 4H, H₂); 0.92 (t, 6H, H₃) |
| c123 | 5.93 (s, 2H, NH); 5.39 (m, 12H, Hvinylic); 2.73 (m, 2H, H₁); 2.20 |
| | (t, 4H, H₂); 1.01 (t, 6H, H₃) |
| c137 | 5.91 (s, 2H, NH); 2.74 (m, 2H, H₁); 2.19 (t, 4H, H₂); 0.93 (t, 6H, |
| | H₃) |

TABLE B

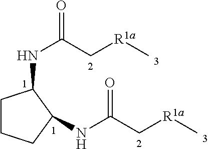

| | ¹H NMR description (300 MHz, CDCl₃, δ ppm) |
|---|---|
| c124 | 6.11 (s, 2H, NH); 5.37 (m, 4H, Hvinylic); 4.11 (m, 2H, H₁); 2.20 |
| | (t, 4H, H₂); 0.91 (t, 6H, H₃) |
| c125 | 6.11 (s, 2H, NH); 4.12 (m, 2H, H₁); 2.18 (m, 4H, H₂); 0.90 (t, 6H, |
| | H₃) |
| c126 | 6.10 (s, 2H, NH); 5.35 (m, 4H, Hvinylic); 4.10 (m, 2H, H₁); 2.16 |
| | (t, 4H, H₂); 0.88 (t, 6H, H₃) |
| c127 | 6.11 (s, 2H, NH); 5.36 (m, 4H, Hvinylic); 4.11 (m, 2H, H₁); 2.19 |
| | (t, 4H, H₂); 0.90 (t, 6H, H₃) |
| c128 | 6.12 (s, 2H, NH); 5.39 (m, 8H, Hvinylic); 4.11 (m, 2H, H₁); 2.18 |
| | (t, 4H, H₂); 0.92 (t, 6H, H₃) |
| c129 | 6.13 (s, 2H, NH); 5.38 (m, 12H, Hvinylic); 4.12 (m, 2H, H₁); 2.20 |
| | (t, 4H, H₂); 1.00 (t, 6H, H₃) |

TABLE C

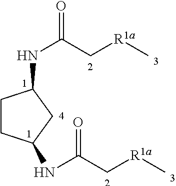

| | ¹H NMR description (300 MHz, CDCl₃, δ ppm) |
|---|---|
| c130 | 6.45 (s, 2H, NH); 5.36 (m, 4H, Hvinylic); 4.08 (m, 2H, H₁); 2.38 |
| | (dt, 1H, H₄); 2.17 (t, 4H, H₂); 0.90 (t, 6H, H₃) |
| c131 | 6.39 (s, 2H, NH); 4.06 (m, 2H, H₁); 2.378 (dt, 1H, H₄); 2.19 (t, |
| | 4H, H₂); 0.91 (t, 6H, H₃) |
| c132 | 6.35 (s, 2H, NH); 4.08 (m, 2H, H₁); 2.38 (dt, 1H, H₄); 2.17 (t, 4H, |
| | H₂); 0.90 (t, 6H, H₃) |
| c133 | 5.36 (m, 4H, Hvinylic); 4.07 (m, 2H, H₁); 2.37 (dt, 1H, H4); 2.17 |
| | (t, 4H, H₂); 0.90 (t, 6H, H₃) |
| c134 | 6.39 (s, 2H, NH); 5.35 (m, 4H, Hvinylic); 4.09 (m, 2H, H₁); 2.38 |
| | (dt, 1H, H4); 2.17 (t, 4H, H₂); 0.90 (t, 6H, H₃) |
| c135 | 6.45 (s, 2H, NH); 5.35 (m, 8H, Hvinylic); 4.08 (m, 2H, H₁); 2.38 |
| | (dt, 1H, H4); 2.17 (t, 4H, H₂); 0.90 (t, 6H, H₃) |
| c136 | 6.43 (s, 2H, NH); 5.36 (m, 12H, Hvinylic); 4.06 (m, 2H, H₁); 2.37 |
| | (dt, 1H, H4); 2.17 (t, 4H, H₂); 0.96 (t, 6H, H₃) |

TABLE F 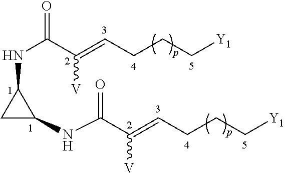
Y₁ and V have the meanings defined above.

| | Deuterated | |
|---|---|---|
| No. | solvent | ¹H NMR description (300 MHz, δ ppm) |
| c101 | DMSO-d6 | 8.15 (s, 2H, NH); 7.93 (s, 4H, —NH₂); 6.55-6.63 (dt, |
| | | 2H, H₃, J_{E} 15 Hz); 5.85 (dd, 2H, H₂, J_{E} 15 Hz); 2.80 |
| | | (m, 2H, H₁); 2.76 (t, 4H, H₅); 2.12 (m, 4H, H₄) |
| c36 | CDCl₃ | 6.58 (s, 2H, NH); 6.04-6.21 (dt, 1H, H₃, J_{Z} 36.9 Hz); |
| | | 5.71-5.84 (dt, 1H, H₃, J_{E} 24.6 Hz); 3.65 (t, 4H, H₅); |
| | | 2.90 (m, 2H, H₁); 2.64 (m, 2H, H₄ E); 2.20 (m, 2H, |
| | | H₄ Z); configuration E/Z |
| c92 | DMSO-d6 | 6.43 (s, 2H, NH); 5.82-6.00 (dt, 1H, H₃, J_{Z} 36.0 Hz); |
| | | 5.67-5.80 (dt, 1H, H₃, J_{E} 27.0 Hz); 3.36 (t, 4H, H₅); |
| | | 3.07 (m, 2H, H₁); 2.11 (m, 4H, H₄); configuration |
| | | E/Z |
| c93 | MeOD | 6.77-6.84 (dt, 2H, H₃, J_{E} 15 Hz); 5.80 (dd, 2H, H₂, |
| | | J_{E} 15 Hz); 3.54 (t, 4H, H₅); 2.68 (m, 2H, H₁); 2.17 |
| | | (m, 4H, H₄) |
| c94 | CDCl₃ | 6.59 (s, 2H, NH); 5.70-5.84 (dt, 2H, H₃, J_{E} 24.0 Hz); |
| | | 3.65 (t, 4H, H₅); 2.89 (m, 2H, H₁); 2.63 (m, 4H, |
| | | H₄ E); configuration E/E |
| c42 | CDCl₃ | 6.58 (s, 2H, NH); 6.05-6.20 (dt, 1H, H₃, J_{Z} 36.9 Hz); |
| | | 5.70-5.82 (dt, 1H, H₃, J_{E} 24.6 Hz); 3.66 (t, 4H, H₅); |
| | | 2.90 (m, 2H, H₁); 2.63 (m, 2H, H₄ E); 2.21 (m, 2H, |
| | | H₄ Z); configuration E/Z |
| c40 | CDCl₃ | 6.56 (s, 2H, NH); 6.05-6.20 (dt, 2H, H₃, J_{Z} 36.9 Hz); |
| | | 3.66 (t, 4H, H₅); 2.91 (m, 2H, H₁); 2.22 (m, 4H, H₄); |
| | | configuration Z/Z |
| c95 | DMSO-d6 | 7.65 (s, 2H, NH); 6.54-6.64 (dt, 2H, H₃, J_{E} 18 Hz); |
| | | 5.91 (dd, 2H, H₂, J_{E} 18 Hz); 3.37 (t, 4H, H₅); 2.81 |
| | | (m, 2H, H₁); 2.10 (m, 4H, H₄) |
| c4 | DMSO-d₆ | 7.66 (s, 2H, NH); 6.54-6.64 (dt, 2H, H₃, J_{E} 15 Hz); |
| | | 5.91 (dd, 2H, H₂, J_{E} 15 Hz); 3.38 (t, 4H, H₅); 2.80 |
| | | (m, 2H, H₁); 2.10 (m, 4H, H₄) |
| c96 | CDCl₃ | 6.65 (s, 2H, NH); 5.69-5.84 (dt, 2H, H₃, J_{E} 27.0 Hz); |
| | | 3.66 (t, 4H, H₅); 2.78 (m, 2H, H₁); 2.62 (m, 4H, |
| | | H₄ E); configuration E/E |
| c97 | MeOD | 6.74-6.82 (dt, 2H, H₃, J_{E} 15 Hz); 5.73 (dd, 2H, H₂, |
| | | J_{E} 15 Hz); 3.54 (t, 4H, H₅); 2.59 (m, 2H, H₁); 2.17 |
| | | (m, 4H, H₄) |
| c1 | DMSO-d₆ | 7.81 (s, 2H, NH); 6.55-6.65 (dt, 2H, H₃, J_{E} 15 Hz); |
| | | 5.93 (dd, 2H, H₂, J_{E} 15 Hz); 3.38 (t, 4H, H₅); 2.83 |
| | | (m, 2H, H₁); 2.10 (m, 4H, H₄) |
| c2 | DMSO-d₆ | 7.67 (s, 2H, NH); 6.55-6.65 (dt, 2H, H₃, J_{E} 15 Hz); |
| | | 5.92 (dd, 2H, H₂, J_{E} 15 Hz); 3.38 (t, 4H, H₅); 2.80 |
| | | (m, 2H, H₁); 2.10 (m, 4H, H₄) |
| c3 | DMSO-d₆ | 7.67 (s, 2H, NH); 6.55-6.64 (dt, 2H, H₃, J_{E} 15 Hz); |
| | | 5.93 (dd, 2H, H₂, J_{E} 15 Hz); 3.40 (t, 4H, H₅); 2.82 |
| | | (m, 2H, H₁); 2.10 (m, 4H, H₄) |
| c5 | DMSO-d₆ | 7.65 (s, 2H, NH); 6.55-6.63 (dt, 2H, H₃, J_{E} 15 Hz); |
| | | 5.91 (dd, 2H, H₂, J_{E} 15 Hz); 3.38 (t, 4H, H₅) 2.81 |
| | | (m, 2H, H₁) |
| c37 | CDCl₃ | 6.58 (s, 2H, NH); 6.04-6.21 (dt, 1H, H₃, J_{Z} 36.9 Hz); |
| | | 5.71-5.84 (dt, 1H, H₃, J_{E} 24.6 Hz); 3.65 (t, 4H, H₅); |
| | | 2.90 (m, 2H, H₁); 2.64 (m, 2H, H₄ E); 2.20 (m, 2H, |
| | | H₄ Z); mixed configuration E/Z |
| c39 | CDCl₃ | 6.58 (s, 2H, NH); 6.04-6.21 (dt, 1H, H₃, J_{Z} 36.9 Hz); |
| | | 5.72-5.81 (dt, 1H, H₃, J_{E} 24.6 Hz); 3.66 (t, 4H, H₅); |
| | | 2.90 (m, 2H, H₁); 2.63 (m, 2H, H₄ E); 2.22 (m, 2H, |
| | | H₄ Z); mixed configuration E/Z |
| c41 | CDCl₃ | 6.60 (s, 2H, NH); 5.72-5.83 (dt, 2H, H₃, J_{E} 24.9 Hz); |
| | | 3.67 (t, 4H, H₅); 2.88 (m, 2H, H₁); 2.61 (m, 4H, H₄); |
| | | configuration E/E |
| c38 | CDCl₃ | 6.58 (s, 2H, NH); 6.04-6.21 (dt, 1H, H₃, J_{Z} 36.9 Hz); |
| | | 5.72-5.81 (dt, 1H, H₃, J_{E} 24.6 Hz); 3.65 (t, 4H, H₅); |
| | | 2.89 (m, 2H, H₁); 2.63 (m, 2H, H₄ E); 2.19 (m, 2H, |
| | | H₄ Z); configuration E/Z |
| c32 | CDCl₃ | 6.81 (dt, 2H, H₃, J_{E} 15 Hz); 6.55 (s, 2H, NH); 5.78 |
| | | (dd, 2H, H₂, J_{E} 15 Hz); 3.38 (t, 2H, H THP); 3.50- |
| | | 3.90 (m, 8H, including H₅); 2.83 (m, 2H, H₁); 2.15 |
| | | (m, 4H, H₄) |
| c86 | CDCl₃ | 6.58 (s, 2H, NH); 5.70-5.84 (dt, 2H, H₃, J_{E} 24.6 Hz); |
| | | 2.89 (m, 2H, H₁); 2.63 (m, 4H, H₄); 0.85 (t, 6H, H₅ |
| | | with X = H); configuration E/E |
| c43 | DMSO-d6 + | 7.96 (s, 2H, NH); 5.65-5.79 (dt, 2H, H₃, J_{E} 24.0 Hz); |
| | D₂O | 3.37 (m, 4H, H₅); 2.95 (m, 2H, H₁); 2.49 (m, 4H, H₄); |
| | | configuration E/E |
| c7 | EtOD | 7.52 (s, 2H, NH); 6.83 (m, 2H, H₃, J_{E} 15 Hz); 5.87 |
| | | (dd, 2H, H₂, J_{E} 15 Hz); 2.80 (m, 2H, H₁); 2.17 (m, |
| | | 4H, H₅) |
| c89 | CDCl₃ | 6.56 (s, 2H, NH); 6.03-6.18 (dt, 1H, H₃, J_{Z} 36.9 Hz); |
| | | 5.67-5.81 (dt, 1H, H₃, J_{E} 24.6 Hz); 4.04 (t, 4H, H₅); |
| | | 2.87 (m, 2H, H₁); 2.61 (m, 2H, H₄ E); 2.14 (m, 2H, |
| | | H₄ Z); 2.04 (s, 6H, —O—CO—CH₃); |
| | | configuration E/Z |
| c35 | CDCl₃ | 6.62 (s, 2H, NH); 5.70-5.80 (dt, 2H, H₃, J_{E} 24.6 Hz); |
| | | 3.66 (t, 4H, H₅); 2.89 (m, 2H, H₁); 2.67 (m, 4H, H₄); |
| | | configuration E/E |
| c103 | CDCl₃ | 7.36-7.70 (m, 10H, H_{aromatic)}; 6.55 (s, 2H, NH); |
| | | 6.05-6.22 (dt, 2H, H₃, J_{Z} 36.9 Hz); 3.66 (t, 4H, H₅); |
| | | 2.91 (m, 2H, H₁); 2.21 (m, 4H, H₄ Z); 1.00 (s, 9H, |
| | | H_{tButyl)}; configuration Z/Z |
| c104 | CDCl₃ | 7.36-7.71 (m, 10H, H_{aromatic)}; 6.56 (s, 2H, NH); |
| | | 6.05-6.22 (dt, 1H, H₃, J_{Z} 36.6 Hz); 5.70-5.83 (dt, 1H, |
| | | H₃, J_{E} 24.3 Hz); 3.67 (t, 4H, H₅); 2.90 (m, 2H, H₁); |
| | | 2.62 (m, 2H, H₄ E); 2.21 (m, 2H, H₄ Z); 1.00 (s, 9H, |
| | | H_{tButyl)}; configuration E/Z |
| c105 | CDCl₃ | 7.36-7.71 (m, 10H, H_{aromatic)}; 6.56 (s, 2H, NH); 5.70- |
| | | 5.83 (dt, 2H, H₃, J_{E} 24.6 Hz); 3.67 (t, 4H, H₅); 2.90 |
| | | (m, 2H, H₁); 2.62 (m, 4H, H₄ E); 1.00 (s, 9H, H_{tButyl)}; |
| | | configuration E/E |
| c102 | DMSO-d6 | 8.05 (s, 2H, NH); 6.77 (s, 2H, —NHBoc); 6.55-6.64 |
| | | (dt, 2H, H₃, J_{E} 18 Hz); 5.80 (dd, 2H, H₂, J_{E} 18 Hz); |
| | | 2.90 (m, 4H, H₅); 2.88 (m, 2H, H₁); 2.10 (m, 4H, H₄); |
| | | 1.36 (s, 9H, —Boc) |
| c110 | CDCl₃ | 6.20 (s, 2H, NH); 6.04-6.16 (dt, 1H, H₃, J_{Z} 36.9 Hz); |
| | | 5.70-5.85 (dt, 1H, H₃, J_{E} 24.6 Hz); 4.58 (m, 2H, H |
| | | foot —THP); 3.35-3.86 (m, 8H including H₅); 2.85 |
| | | (m, 2H, H₁); 2.63 (m, 2H, H₄ E); 2.22 (m, 2H, H₄ Z); |
| | | configuration E/Z |
| c111 | CDCl₃ | 6.56 (s, 2H, NH); 6.05-6.22 (dt, 2H, H₃, J_{Z} 36.9 Hz); |
| | | 4.58 (m, 2H, H foot —THP); 3.36-3.87 (m, 8H |
| | | including H₅); 2.91 (m, 2H, H₁); 2.22 (m, 4H, H₄ Z); |
| | | configuration Z/Z |
| c112 | CDCl₃ | 6.62 (s, 2H, NH); 5.95-6.13 (dt, 2H, H₃, J_{E} 27.3 Hz); |
| | | 3.66 (t, 4H, H₅); 2.89 (m, 2H, H₁); 2.63 (m, 4H, |
| | | H₄ E); configuration E/E |
| c113 | CDCl₃ | 6.63 (s, 2H, NH); 6.04-6.21 (dt, 1H, H₃, J_{Z} 36.6 Hz); |
| | | 5.71-5.85 (dt, 1H, H₃, J_{E} 24.6 Hz); 3.65 (t, 4H, H₅); |
| | | 2.89 (m, 2H, H₁); 2.63 (m, 2H, H₄ E); 2.22 (m, 2H, |
| | | H₄ Z); configuration E/E |
| c109 | CDCl₃ | 5.95-6.13 (dt, 2H, H₃, J_{Z} 36.6 Hz); 3.56 (t, 4H, H₅); |
| | | 2.86 (m, 2H, H₁); 2.16 (m, 4H, H₄ Z); |
| | | configuration Z/Z |

TABLE G

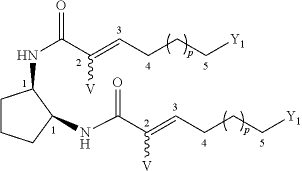

| | Deuterated | |
|---|---|---|
| No. | solvent | ¹H NMR description (300 MHz, δ ppm) |
| c18 | CDCl₃ | 6.83 (dt, 2H, H₃, J_{E} 14.5 Hz); 6.42 (s, 2H, NH); 5.80 |
| | | (dd, 2H, H₂, J_{E} 14.5 Hz); 4.20 (m, 2H, H₁); 3.66 (t, |
| | | 4H, H₅) |
| c53 | CDCl₃ | 6.55 (s, 2H, NH); 6.02-6.18 (dt, 1H, H₃, J_{Z} 36.9 Hz); |
| | | 5.69-5.80 (dt, 1H, H₃, J_{E} 24.6 Hz); 4.31 (m, 2H, H₁); |
| | | 3.66 (t, 4H, H₅); 2.61 (m, 2H, H₄ E); 2.19 (m, 2H, |
| | | H₄ Z); configuration E/Z |
| c33 | CDCl₃ | 6.78-6.87 (dt, 2H, H₃, J_{E} 15.0 Hz); 6.17 (s, 2H, NH); |
| | | 5.79 (dd, 2H, H₂, J_{E} 15.0 Hz); 4.59 (t, 2H, H THP); |
| | | 4.19 (m, 2H, H₁); 3.36-3.91 (m, 8H, including H₅) |
| c75 | CDCl₃ | 6.55 (s, 2H, NH); 6.03-6.17 (dt, 1H, H₃, J_{Z} 36.9 Hz); |
| | | 5.71-5.80 (dt, 1H, H₃, J_{E} 24.6 Hz); 4.59 (t, 2H, |
| | | H THP); 4.31 (m, 2H, H₁); 3.36-3.93 (m, 8H, |
| | | including H₅); 2.63 (m, 2H, H₄ E); 2.20 (m, 2H, |
| | | H₄ Z); configuration E/Z |
| c19 | DMSO-d6 | 7.32 (sd, 2H, NH); 6.54-6.61 (dt, 2H, H₃, J_{E} 15.3 Hz); |
| | | 5.80 (dd, 2H, H₂, J_{E} 15.3 Hz); 4.16 (m, 2H, H₁); 3.39 |
| | | (t, 4H, H₅) |
| c54 | CDCl₃ | 6.60 (s, 2H, NH); 6.02-6.18 (dt, 1H, H₃, J_{Z} 36.9 Hz); |
| | | 5.69-5.80 (dt, 1H, H₃, J_{E} 24.6 Hz); 4.31 (m, 2H, H₁); |
| | | 3.66 (t, 4H, H₅); 2.62 (m, 2H, H₄ E); 2.20 (m, 2H, |
| | | H₄ Z); configuration E/Z |
| c50 | CDCl₃ | 6.60 (s, 2H, NH); 6.02-6.19 (dt, 2H, H₃, J_{Z} 36.9 Hz); |
| | | 4.30 (m, 2H, H₁); 3.66 (t, 4H, H₅); 2.22 (m, 4H, |
| | | H₄ Z); configuration Z/Z |
| c52 | CDCl₃ | 6.57 (s, 2H, NH); 6.02-6.20 (dt, 1H, H₃, J_{Z} 36.9 Hz); |
| | | 5.69-5.83 (dt, 1H, H₃, J_{E} 24.6 Hz); 4.30 (m, 2H, H₁); |
| | | 3.66 (t, 4H, H₅); 2.61 (m, 2H, H₄ E); 2.21 (m, 2H, |
| | | H₄ Z); configuration E/Z |
| c51 | CDCl₃ | 6.55 (s, 2H, NH); 5.67-5.80 (dt, 2H, H₃, J_{E} 24.6 Hz); |
| | | 4.30 (m, 2H, H₁); 3.66 (t, 4H, H₅); 2.61 (m, 4H, |
| | | H₄ E); configuration E/E |
| c34 | CDCl₃ | 6.80-6.88 (dt, 2H, H₃, J_{E} 15.3 Hz); 6.54 (s, 2H, NH); |
| | | 5.78 (dd, 2H, H₂, J_{E} 15.3 Hz); 4.59 (t, 2H, H THP); |
| | | 4.15 (m, 2H, H₁); 3.36-3.89 (m, 8H, including H₄) |
| c65 | CDCl₃ | 6.65 (s, 2H, NH); 6.03-6.18 (dt, 2H, H₃, J_{Z} 36.9 Hz); |
| | | 4.59 (t, 2H, H THP); 4.31 (m, 2H, H₁); 3.35-3.89 (m, |
| | | 8H, including H₅); 2.22 (m, 4H, H₄ Z); configuration |
| | | Z/Z |
| c66 | CDCl₃ | 6.65 (s, 2H, NH); 6.03-6.18 (dt, 1H, H₃, J_{Z} 36.9 Hz); |
| | | 5.69-5.80 (dt, 1H, H₃, J_{E} 24.6 Hz); 4.59 (t, 2H, |
| | | H THP); 4.31 (m, 2H, H₁); 3.37-3.88 (m, 8H, |
| | | including H₅); 2.62 (m, 2H, H₄ E); 2.20 (m, 2H, |
| | | H₄ Z); configuration E/Z |
| c67 | CDCl₃ | 6.65 (s, 2H, NH); 5.69-5.80 (dt, 2H, H₃, J_{E} 24.6 Hz); |
| | | 4.59 (t, 2H, H THP); 4.31 (m, 2H, H₁); 3.37-3.88 |
| | | (m, 8H, including H₅); 2.62 (m, 4H, H₄ E); |
| | | configuration E/E |
| c14 | CDCl₃ | 6.74 (dt, 2H, H₃, J_{E} 15.0 Hz); 6.11 (s, 2H, NH); 5.71 |
| | | (dd, 2H, H₂, J_{E} 15.0 Hz); 4.11 (m, 2H, H₁); 3.576 |
| | | (t, 4H, H₅) |
| c31 | CDCl₃ | 6.81-6.86 (dt, 2H, H₃, J_{E} 15.0 Hz); 6.16 (s, 2H, NH); |
| | | 5.78 (dd, 2H, H₂, J_{E} 15.0 Hz); 4.60 (t, 2H, H THP); |
| | | 4.19 (m, 2H, H₁); 3.38-3.89 (m, 8H, including H₅) |
| c17 | CDCl₃ | 6.74 (dt, 2H, H₃, J_{E} 15.3 Hz); 6.11 (s, 2H, NH); 5.70 |
| | | (dd, 2H, H₂, J_{E} 15.3 Hz); 4.10 (m, 2H, H₁); 3.57 (t, |
| | | 4H, H₅) |
| c45 | DMSO-d6 | 7.90 (s, 2H, NH); 5.78-5.97 (dt, 2H, H₃, J_{Z} 36.9 Hz); |
| | | 4.25 (m, 2H, H₁); 3.37 (t, 4H, H₅); 2.12 (m, 4H, H₄ Z); |
| | | configuration Z/Z |
| c46 | DMSO-d6 | 7.90 (s, 2H, NH); 5.81-5.98 (dt, 1H, H₃, J_{Z} 36.9 Hz); |
| | | 5.67-5.80 (dt, 2H, H₃, J_{E} 24.6 Hz); 4.23 (m, 2H, H₁); |
| | | 3.37 (t, 4H, H₅); 2.44 (m, 2H, H₄ E); 2.12 (m, 2H, |
| | | H₄ Z); configuration E/Z |
| c47 | DMSO-d6 | 7.90 (s, 2H, NH); 5.67-5.81 (dt, 2H, H₃, J_{E} 24.6 Hz); |
| | | 4.23 (m, 2H, H₁); 3.37 (t, 4H, H₅); 2.43 (m, 4H, |
| | | H₄ E); configuration E/E |
| c79 | CDCl₃ | 6.77-6.87 (dt, 2H, H₃, J_{E} 15.3 Hz); 6.26 (s, 2H, NH); |
| | | 5.75-5.81 (dd, 2H, H₂, J_{E} 15.3 Hz); 4.20 (m, 2H, H₁); |
| | | 0.89 (t, 6H, H₅ with X = H) |
| c27 | CDCl₃ | 6.70-6.77 (dt, 2H, H₃, J_{E} 15.3 Hz); 6.18 (s, 2H, NH); |
| | | 5.70 (dd, 2H, H₂, J_{E} 15.3 Hz); 4.50 (t, 2H, H THP); |
| | | 4.11 (m, 2H, H₁); 3.27-3.80 (m, 8H, including H₅) |
| c12 | CDCl₃ | 6.82 (dt, 2H, H₃, J_{E} 15.3 Hz); 6.20 (s, 2H, NH); 5.80 |
| | | (dd, 2H, H₂, J_{E} 15.3 Hz); 4.20 (m, 2H, H₁); 3.66 (t, |
| | | 4H, H₅) |
| c28 | CDCl₃ | 6.78-6.88 (dt, 2H, H₃, J_{E} 15.3 Hz); 6.18 (s, 2H, NH); |
| | | 5.78 (dd, 2H, H₂, J_{E} 15.3 Hz); 4.59 (t, 2H, H THP); |
| | | 4.20 (m, 2H, H₁); 3.36-3.92 (m, 8H, including H₅) |
| c13 | CDCl₃ | 6.82 (dt, 2H, H₃, J_{E} 15.0 Hz); 6.22 (s, 2H, NH); 5.79 |
| | | (dd, 2H, H₂, J_{E} 15.0 Hz); 4.19 (m, 2H, H₁); 3.66 (t, |
| | | 4H, H₅) |
| c15 | DMSO-d6 | 7.46 (sd, 2H, NH); 6.50-6.60 (dt, 2H, H₃, J_{E} 15.0 Hz); |
| | | 5.87 (dd, 2H, H₂, J_{E} 15.0 Hz); 4.12 (m, 2H, H₁); 3.37 (t, |
| | | 4H, H₅) |
| c71 | CDCl₃ | 6.55 (s, 2H, NH); 6.03-6.18 (dt, 1H, H₃, J_{Z} 36.9 Hz); |
| | | 5.69-5.80 (dt, 1H, H₃, J_{E} 24.6 Hz); 4.59 (t, 2H, |
| | | H THP); 4.31 (m, 2H, H₁); 3.37-3.89 (m, 8H, |
| | | including H₅); 2.62 (m, 2H, H₄ E); 2.20 (m, 2H, H₄ Z); |
| | | configuration E/Z |
| c72 | CDCl₃ | 6.54 (s, 2H, NH); 5.67-5.80 (dt, 2H, H₃, J_{E} 24.6 Hz); |
| | | 4.59 (t, 2H, H THP); 4.31 (m, 2H, H₁); 3.35-3.92 (m, |
| | | 8H, including H₅); 2.62 (m, 4H, H₄ E); configuration |
| | | E/E |
| c73 | CDCl₃ | 6.55 (s, 2H, NH); 6.03-6.18 (dt, 2H, H₃, J_{Z} 36.9 Hz); |
| | | 4.59 (t, 2H, H THP); 4.31 (m, 2H, H₁); 3.37-3.89 (m, |
| | | 8H, including H₅); 2.20 (m, 4H, H₄ Z); configuration |
| | | Z/Z |
| c74 | CDCl₃ | 6.55 (s, 2H, NH); 6.03-6.18 (dt, 1H, H₃, J_{Z} 36.9 Hz); |
| | | 5.69-5.80 (dt, 1H, H₃, J_{E} 24.6 Hz); 4.59 (t, 2H, |
| | | H THP); 4.25 (m, 2H, H₁); 3.37-3.89 (m, 8H, |
| | | including H₅); 2.63 (m, 2H, H₄ E); 2.20 (m, 2H, |
| | | H₄ Z); configuration E/Z |
| c49 | CDCl₃ | 6.53 (s, 2H, NH); 5.67-5.81 (dt, 2H, H₃, J_{E} 24.6 Hz); |
| | | 4.30 (m, 2H, H₁); 3.65 (t, 4H, H₅); 2.63 (m, 4H, |
| | | H₄ E); configuration E/E |
| c81 | CDCl₃ | 6.59 (s, 2H, NH); 6.02-6.19 (dt, 2H, H₃, J_{Z} 36.9 Hz); |
| | | 4.31 (m, 2H, H₁); 2.22 (m, 4H, H₄ Z); 0.90 (t, 6H, H₅ |
| | | with X = H); configuration Z/Z |
| c82 | CDCl₃ | 6.55 (d, 2H, NH); 6.01-6.18 (dt, 1H, H₃, J_{Z} 36.9 Hz); |
| | | 5.68-5.82 (dt, 1H, H₃, J_{E} 24.6 Hz); 4.31 (m, 2H, H₁); |
| | | 2.63 (m, 2H, H₄ E); 2.20 (m, 2H, H₄ Z); 0.90 |
| | | (t, 6H, H₅ with X = H); configuration E/Z |
| c100 | CDCl₃ | 6.54 (s, 2H, NH); 5.32-5.81 (dt, 2H, H₃, J_{E} 24.9 Hz); |
| | | 4.31 (m, 2H, H₁); 2.61 (m, 4H, H₄ E); 0.90 (t, 6H, |
| | | H₅ with X = H); configuration E/E |
| c16 | DMSO-d6 | 7.46 (d, 2H, NH); 6.50-6.60 (dt, 2H, H₃, J_{E} 15.0 Hz); |
| | | 5.88 (dd, 2H, H₂, J_{E} 15.0 Hz); 4.11 (m, 2H, H₁); 3.37 |
| | | (t, 4H, H₅) |
| c107 | CDCl₃ | 6.51 (s, 2H, NH); 5.94-6.11 (dt, 2H, H₃, J_{Z} 36.9 Hz); |
| | | 4.51 (t, 2H, H THP); 4.22 (m, 2H, H₁); 3.27-3.82 (m, |
| | | 8H, including H₅); 2.15 (m, 4H, H₄ Z); configuration |
| | | Z/Z |
| c117 | CDCl₃ | 6.54 (s, 2H, NH); 5.67-5.81 (dt, 2H, H₃, J_{E} 24.9 Hz); |
| | | 4.32 (m, 2H, H₁); 3.65 (t, 4H, H₅); 2.63 (m, 4H, H₄ E); |
| | | configuration E/E |
| c118 | CDCl₃ | 6.59 (s, 2H, NH); 6.01-6.19 (dt, 2H, H₃, J_{Z} 36.9 Hz); |
| | | 4.32 (m, 2H, H₁); 3.66 (t, 4H, H₅); 2.22 (m, 4H, H₄ Z); |
| | | configuration Z/Z |

TABLE H

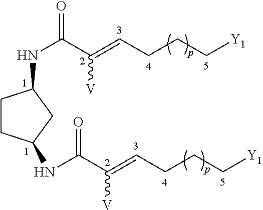

| | Deuterated | |
|---|---|---|
| No. | solvent | ¹H NMR description (300 MHz, δ ppm) |
| c61 | CDCl₃ | 6.77 (bs, 2H, NH); 6.03-6.16 (dt, 1H, H₃, J_{Z} 36.9 Hz); |
| | | 5.69-5.79 (dt, 1H, H₃, J_{E} 24.9 Hz); 4.24 (m, 2H, H₁); |
| | | 3.65 (t, 4H, H₅); 2.64 (m, 2H, H₄ E); 2.19 (m, 2H, |
| | | H₄ Z); configuration E/Z |
| c60 | CDCl₃ | 6.85 (bs, 2H, NH); 6.03-6.16 (dt, 1H, H₃, J_{Z} 36.9 Hz); |
| | | 5.69-5.79 (dt, 1H, H₃, J_{E} 24.9 Hz); 4.24 (m, 2H, H₁); |
| | | 3.66 (t, 4H, H₅); 2.64 (m, 2H, H₄ E); 2.22 (m, 2H, |
| | | H₄ Z); configuration E/Z |
| c76 | CDCl₃ | 6.90 (s, 2H, NH); 6.01-6.19 (dt, 2H, H₃, J_{Z} 37.2 Hz); |
| | | 4.59 (t, 2H, H THP); 4.25 (m, 2H, H₁); 3.36-3.90 |
| | | (m, 8H, including H₅); 2.20 (m, 4H, H₄ Z); |
| | | configuration Z/Z |
| c77 | CDCl₃ | 6.80 (s, 2H, NH); 6.02-6.16 (dt, 1H, H₃, J_{Z} 36.9 Hz); |
| | | 5.70-5.80 (dt, 1H, H₃, J_{E} 24.6 Hz); 4.59 (t, 2H, |
| | | H THP); 4.24 (m, 2H, H₁); 3.36-3.90 (m, 8H, |
| | | including H₅); 2.63 (m, 2H, H₄ E); 2.19 (m, 2H, |
| | | H₄ Z); configuration E/Z |
| c78 | CDCl₃ | 6.73 (s, 2H, NH); 5.67-5.80 (dt, 2H, H₃, J_{E} 24.6 Hz); |
| | | 4.59 (t, 2H, H THP); 4.24 (m, 2H, H₁); 3.36-3.91 |
| | | (m, 8H, including H₅); 2.65 (m, 4H, H₄ E); |
| | | configuration E/E |
| c68 | CDCl₃ | 6.92 (s, 2H, NH); 6.00-6.18 (dt, 2H, H₃, J_{Z} 37.2 Hz); |
| | | 4.59 (t, 2H, H THP); 4.23 (m, 2H, H₁); 3.35-3.88 |
| | | (m, 8H, including H₅); 2.18 (m, 4H, H₄ Z); |
| | | configuration Z/Z |
| c69 | CDCl₃ | 6.80 (s, 2H, NH); 6.00-6.15 (dt, 1H, H₃, J_{Z} 36.9 Hz); |
| | | 5.68-5.78 (dt, 1H, H₃, J_{E} 24.9 Hz); 4.59 (t, 2H, |
| | | H THP); 4.24 (m, 2H, H₁); 3.36-3.88 (m, 8H, |
| | | including H₅); 2.62 (m, 2H, H₄ E); 2.19 (m, 2H, |
| | | H₄ Z); configuration E/Z |
| c70 | CDCl₃ | 6.74 (s, 2H, NH); 5.65-5.80 (dt, 2H, H₃, J_{E} 25.2 Hz); |
| | | 4.59 (t, 2H, H THP); 4.23 (m, 2H, H₁); 3.35-3.88 |
| | | (m, 8H, including H₅); 2.64 (m, 4H, H₄ E); |
| | | configuration E/E |
| c55 | DMSO-d6 | 8.31 (d, 2H, NH); 5.82-6.00 (dt, 2H, H₃, J_{Z} 36.6 Hz); |
| | | 4.12 (m, 2H, H₁); 3.36 (t, 4H, H₅); 2.12 (m, 4H, H₄ Z); |
| | | configuration Z/Z |
| c56 | DMSO-d6 | 8.31 (d, 2H, NH); 5.82-6.00 (dt, 1H, H₃, J_{Z} 36.6 Hz); |
| | | 5.67-5.5.81 (dt, 1H, H₃, J_{E} 24.9 Hz); 4.12 (m, 2H, H₁); |
| | | 3.36 (t, 4H, H₅); 2.10-2.26 (m, 4H, H₄ Z and E); |
| | | configuration E/Z |
| c57 | DMSO-d6 | 8.30 (d, 2H, NH); 5.67-5.81 (dt, 2H, H₃, J_{E} 24.9 Hz); |
| | | 4.12 (m, 2H, H₁); 3.36 (t, 4H, H₅); 2.22 (m, 4H, H₄ E); |
| | | configuration E/E |
| c22 | DMSO-d6 | 7.93 (d, 2H, NH); 6.54-6.63 (dt, 2H, H₃, J_{E} 15.6 Hz); |
| | | 5.83-5.88 (dd, 2H, H₂, J_{E} 15.6 Hz); 4.01 (m, 2H, H₁); |
| | | 3.37 (t, 4H, H₅); 2.12 (m, 4H, H₄) |
| c30 | CDCl₃ | 6.78-6.87 (dt, 2H, H₃, J_{E} 15.0 Hz); 6.54 (d, 2H, NH); |
| | | 5.83-5.88 (dd, 2H, H₂, J_{E} 15.0 Hz); 4.59 (m, 2H, |
| | | H —THP); 4.15 (m, 2H, H₁); 3.36-3.89 (m, 8H, |
| | | including H₅); 2.17 (m, 4H, H₄) |
| c24 | CDCl₃ | 6.78-6.93 (dt, 2H, H₃, J_{E} 15.0 Hz); 5.79-5.84 (dd, |
| | | 2H, H₂, J_{E} 15.0 Hz); 4.13 (m, 2H, H₁); 3.66 (t, 4H, |
| | | H₅); 2.18 (m, 4H, H₄) |
| c80 | CDCl₃ | 6.81-6.91 (dt, 2H, H₃, J_{E} 15.0 Hz); 6.55 (s, 2H, NH); |
| | | 5.77-5.82 (dd, 2H, H₂, J_{E} 15.0 Hz); 4.17 (m, 2H, H₁); |
| | | 0.91 (t, 6H, H₅ with X = H) |
| c20 | DMSO-d6 | 7.95 (d, 2H, NH); 6.54-6.63 (dt, 2H, H₃, J_{E} 15.0 Hz); |
| | | 5.83-5.88 (dd, 2H, H₂, J_{E} 15.0 Hz); 4.02 (m, 2H, H₁); |
| | | 3.37 (t, 4H, H₅); 2.13 (m, 4H, H₄) |
| c21 | CDCl₃ | 6.78-6.93 (dt, 2H, H₃, J_{E} 15.0 Hz); 5.79-5.84 (dd, 2H, |
| | | H₂, J_{E} 15.0 Hz); 4.17 (m, 2H, H₁); 3.64 (t, 4H, H₅); |
| | | 2.21 (m, 4H, H₄) |
| c29 | DMSO-d6 | 7.92 (d, 2H, NH); 6.54-6.63 (dt, 2H, H₃, J_{E} 15.6 Hz); |
| | | 5.83-5.88 (dd, 2H, H₂, J_{E} 15.6 Hz); 4.52 (m, 2H, |
| | | H —THP); 4.01 (m, 2H, H₁); 3.30-3.80 (m, 8H, |
| | | including H₅); 2.12 (m, 4H, H₄) |
| c23 | DMSO-d6 | 7.93 (d, 2H, NH); 6.54-6.63 (dt, 2H, H₃, J_{E} 15.6 Hz); |
| | | 5.83-5.88 (dd, 2H, H₂, J_{E} 15.6 Hz); 4.02 (m, 2H, H₁); |
| | | 3.36 (t, 4H, H₅); 2.14 (m, 4H, H₄) |
| c73 | CDCl₃ | 6.85 (s, 2H, NH); 5.99-6.18 (dt, 2H, H₃, J_{Z} 36.9 Hz); |
| | | 4.59 (t, 2H, H THP); 4.25 (m, 2H, H₁); 3.35-3.90 (m, |
| | | 8H, including H₅); 2.20 (m, 4H, H₄ Z); |
| | | configuration Z/Z |
| c74 | CDCl₃ | 6.85 (s, 2H, NH); 5.99-6.18 (dt, 1H, H₃, J_{Z} 36.9 Hz); |
| | | 5.63-5.82 (dt, 1H, H₃, J_{E} 24.6 Hz); 4.59 (t, 2H, |
| | | H THP); 4.25 (m, 2H, H₁); 3.35-3.90 (m, 8H, |
| | | including H₅); 2.65 (m, 2H, H₄ E); 2.20 (m, 2H, |
| | | H₄ Z); configuration E/Z |
| c58 | CDCl₃ | 6.94 (s, 2H, NH); 5.94-6.08 (dt, 2H, H₃, J_{Z} 36.9 Hz); |
| | | 4.12 (m, 2H, H₁); 3.57 (t, 4H, H₅); 2.12 (m, 4H, H₄ Z); |
| | | configuration Z/Z |
| c59 | CDCl₃ | 6.90 (bs, 2H, NH); 5.94-6.09 (dt, 1H, H₃, J_{Z} 37.2 Hz); |
| | | 5.58-5.72 (dt, 1H, H₃, J_{E} 25.2 Hz); 4.15 (m, 2H, H₁); |
| | | 3.57 (t, 4H, H₅); 2.54 (m, 2H, H₄ E); 2.11 (m, 2H, |
| | | H₄ Z); configuration E/Z |
| c99 | CDCl₃ | 6.79-6.86 (dt, 2H, H₃, J_{E} 15.3 Hz); 6.69 (d, 2H, NH); |
| | | 5.76-5.82 (dd, 2H, H₂, J_{E} 15.3 Hz); 4.57 (m, 2H, |
| | | H —THP); 4.15 (m, 2H, H₁); 3.36-3.88 (m, 8H, |
| | | including H₅); 2.18 (m, 4H, H₄) |
| c83 | CDCl₃ | 6.90 (s, 2H, NH); 6.02-6.19 (dt, 2H, H₃, J_{Z} 37.2 Hz); |
| | | 4.25 (m, 2H, H₁); 2.22 (m, 4H, H₄ Z); 0.90 (t, 6H, H₅); |
| | | configuration Z/Z |
| c84 | CDCl₃ | 6.81 (s, 2H, NH); 6.00-6.18 (dt, 1H, H₃, J_{Z} 37.2 Hz); |
| | | 5.67-5.81 (dt, 1H, H₃, J_{E} 24.9 Hz); 4.24 (m, 2H, H₁); |
| | | 2.65 (m, 2H, H₄ E); 2.22 (m, 2H, H₄ Z); 0.90 (t, 6H, |
| | | H₅); configuration E/Z |
| c85 | CDCl₃ | 6.73 (s, 2H, NH); 5.67-5.81 (dt, 2H, H₃, J_{E} 24.9 Hz); |
| | | 4.25 (m, 2H, H₁); 2.65 (m, 4H, H₄ E); 0.91 (t, 6H, |
| | | H₅); configuration E/E |
| c90 | CDCl₃ | 6.57 (s, 2H, NH); 6.03-6.21 (dt, 1H, H₃, J_{Z} 36.9 Hz); |
| | | 5.70-5.84 (dt, 1H, H₃, J_{E} 24.9 Hz); 4.07 (t, 4H, H₅); |
| | | 2.91 (m, 2H, H₁); 2.64 (m, 2H, H₄ E); 2.19 (m, 2H, |
| | | H₄ Z); 2.06 (s, 6H, —O—CO—CH₃); |
| | | configuration E/Z |
| c106 | CDCl₃ | 6.75 (s, 2H, NH); 5.67-5.80 (dt, 2H, H₃, J_{E} 24.6 Hz); |
| | | 4.59 (t, 2H, H THP); 4.21 (m, 2H, H₁); 3.36-3.87 (m, |
| | | 8H, including H₅); 2.67 (m, 4H, H₄ E); |
| | | configuration E/E |
| c114 | CDCl₃ | 6.90 (d, 2H, NH); 5.67-5.81 (dt, 2H, H₃, J_{E} 24.9 Hz); |
| | | 4.21 (m, 2H, H₁); 4.06 (t, 4H, H₅); 2.63 (m, 4H, |
| | | H₄ E); configuration E/E |
| c115 | CDCl₃ | 6.90 (d, 2H, NH); 6.00-6.18 (dt, 1H, H₃, J_{Z} 36.6 Hz); |
| | | 5.68-5.82 (dt, 1H, H₃, J_{E} 24.9 Hz); 4.22 (m, 2H, H₁); |
| | | 3.66 (t, 4H, H₅); 2.63 (m, 2H, H₄ E); 2.22 (m, 2H, |
| | | H₄ Z); configuration E/Z |
| c116 | CDCl₃ | 6.97 (d, 2H, NH); 6.06-6.19 (dt, 2H, H₃, J_{Z} 36.6 Hz); |
| | | 4.22 (m, 2H, H₁); 3.66 (t, 4H, H₅); 2.23 (m, 4H, H₄ Z); |
| | | configuration Z/Z |

### Example 27

### Screening of Anti-Tyrosinase Activity

The tests were carried out by reaction with DOPA oxidase on separated epidermis, compared with a DOPA control and with kojic acid at 0.06%. The products are tested in solution at 30 μg/mL in DMSO.

Procedure:

Epidermis samples originating from a frozen abdominoplasty (woman 33 years old) were separated by incubation in 2N NaBr for 1.0 h at 37° C.
They were then fixed in a buffered formolized fixing agent, rinsed and put in contact with the volume/volume mixture: solutions of L-DOPA/test formulation.
After incubation, they were rinsed and mounted between slide and cover slip with mounting liquid of the Aquatex type.
Observation was by optical microscopy with ×10 objective.
Images were obtained with a tri CCD Sony DXC 390P camera and stored using the Leica IM1000 data archiving software.
For each batch, several microscope fields were analysed using the LEICA QWin image analysis software.
For each field, the DOPA positive melanocytes were counted and the area of the zone was measured to determine the melanocyte count per mm².

Results:

The observations of the epidermis samples tested with the different solutions of products dissolved in DMSO live the following results:

| | Variation/ |
|---|---|
| Compound No. | control |
| Kojic acid | −81 |
| c40 | −61 |
| c59 | −56 |
| c42 | −55 |
| c129 | −52 |
| c93 | −48 |
| c127 | −45 |
| c79 | −45 |
| Ref 4 | −36 |
| 2-Fluoro-14- | |
| hydroxy- | |
| tetradec-2- | |
| enoic acid | |
| Ref 5 | −29 |
| (E)-15- | |
| Hydroxy- | |
| pentadec-2- | |
| enoic acid | |
| Ref 6 | −24 |
| (E)-12- | |
| Hydroxy- | |
| dodec-2-enoic | |
| acid | |
| Ref 1 | −16 |
| (E)-18- | |
| Hydroxy- | |
| octadec-2- | |
| enoic acid | |
| Ref 3 | −15 |
| (E)-14- | |
| Hydroxy- | |
| tetradec-2- | |
| enoic acid | |

The variations are larger with compounds 40, 59 and 42 than with the compounds designated ref 1, ref 3, ref 4, ref 5 and ref 6, and indicated in the above table.
These compounds are therefore more effective than the reference compounds for limiting tyrosinase activity.

### Example 28

### Screening of Anti-Melanogenesis Activity

This was carried out for compounds in solution in DMSO. The synthesis of melanin was measured in a model of B16 melanocytes stimulated with a stable derivative of α-MSH (natural hormone that stimulates melanogenesis: Melanocyte Stimulating Hormone): NDP-MSH ([Nle⁴, DPhe7]-α-MSH).

Culture and Treatments:

Melanocytes were seeded on a 96-well plate and cultured for 24 h (37° C., 5% CO₂, DMEM 1 g/L glucose without phenol red supplemented with glucose 3 g/L, L-glutamine 2 mM, penicillin 50 U/mL, streptomycin 50 μg/mL, fetal calf serum (FCS) 10%). After incubation, the culture medium was then replaced with supplemented or unsupplemented culture medium (non-stimulated control) with a stable derivative of α-MSH and with or without (controls) the test compounds or the reference (kojic acid at 25, 100, 400, 800 μg/mL). Each experimental condition was carried out with n=3, apart from the controls carried out with n=6. The cells were then incubated for 72 h. Wells without cells received in parallel the same quantities of medium, supplemented or not with NDP-MSH and with or without the test compounds or the reference in order to quantify the background noise associated with the presence of the compounds.

Melanin Assay

At the end of 72 hours of incubation, the total melanin (intra- and extracellular) was quantified by measuring the absorbance of each sample at 405 nm (direct reading of the culture plates) against a standard range of melanin (melanin concentrations tested from 0.78 to 100 μg/mL).
The background noise, measured in the wells without cells, was subtracted from the values measured so that only the effect connected with the production of melanin is taken into account, without including any interference connected with the presence of the compounds. The results were expressed in percentage of melanin relative to the control as well as in percentage inhibition.

Evaluation of the Viability of the Cells—Test of Reduction of MTT

At the end of the treatment, the cells were incubated in the presence of MTT (3-(4,5-dimethylthiazol-2-yl)-2,5-diphenyl tetrazolium bromide), the conversion of which to blue crystals of formazan is proportional to the activity of succinate dehydrogenase (mitochondrial enzyme). After disruption of the cells, the formazan was dissolved in DMSO medium and the optical density (OD), representative of the number of live cells and of their metabolic reactivity, was measured with a microplate reader at 540 nm (VERSAmax, Molecular Devices).

| Name and | | Normalized | Viability (MTT) |
|---|---|---|---|
| concentrations tested | | data | % stimulated |
| (μM) | | inhibition (%) | control |
| Stimulated | 10⁻⁷M | 0 | 100 |
| control | | | |
| Non stimulated | — | 100 | 103 |
| control | | | |
| Kojic acid | 25 μg/mL | 59 | 103 |
| | 100 μg/mL | 83 | 99 |
| | 400 μg/mL | 93 | 96 |
| c59 | 1 | 0 | 108 |
| | 3 | 9 | 90 |
| | 10 | 38 | 88 |
| c40 | 10 | −1 | 89 |
| | 30 | −6 | 85 |
| | 100 | 40 | 86 |
| c44 | 1 | −11 | 87 |
| | 3 | 36 | 88 |
| | 10 | 69 | 91 |
| c69 | 30 | 116 | 87 |
| | 100 | 112 | 78 |
| c66 | 30 | 119 | 96 |
| | 100 | 118 | 93 |
| c67 | 30 | 119 | 71 |
| | 100 | 116 | 81 |
| c70 | 30 | 115 | 90 |
| | 100 | 116 | 86 |
| c74 | 30 | 110 | 78 |
| | 100 | 111 | 81 |
| c106 | 30 | 114 | 73 |
| | 100 | 119 | 36 |
| c149 | 30 | 108 | 87 |
| | 100 | 120 | 28 |
| c110 | 30 | 104 | 83 |
| | 100 | 120 | 0 |
| c71 | 30 | 93 | 116 |
| | 100 | 109 | 104 |
| c72 | 30 | 87 | 102 |
| | 100 | 112 | 86 |
| c8 | 30 | 86 | 86 |
| | 100 | 115 | 77 |
| c60 | 30 | 83 | 105 |
| | 100 | 97 | 101 |
| c49 | 30 | 83 | 83 |
| | 100 | 118 | 0 |
| c108 | 30 | 73 | 75 |
| | 100 | 105 | 80 |
| c59 | 30 | 71 | 82 |
| | 100 | 118 | 2 |
| c107 | 30 | 66 | 95 |
| | 100 | 120 | 26 |
| c58 | 30 | 62 | 101 |
| | 100 | 83 | 98 |
| c111 | 30 | 25 | 98 |
| | 100 | 108 | 57 |
| c68 | 30 | 95 | 64 |
| | 100 | 108 | 50 |
| c140 | 10 | 112 | 45 |
| | 30 | 123 | 0 |
| Ref1: | 30 | 0 | 79 |
| (E)-18- | 100 | 115 | 63 |
| Hydroxy- | | | |
| octadec-2-enoic | | | |
| acid | | | |
| c161 | 30 | 17 | 89 |
| | 100 | 114 | 75 |
| c27 | 30 | 12 | 89 |
| | 100 | 110 | 71 |
| c122 | 30 | 8 | 75 |
| | 100 | 109 | 79 |
| c40 | 30 | 14 | 94 |
| | 100 | 98 | 94 |
| c115 | 30 | 0 | 94 |
| | 100 | 95 | 97 |
| c128 | 30 | 9 | 88 |
| | 100 | 93 | 75 |
| c37 | 30 | 0 | 77 |
| | 100 | 92 | 83 |
| c135 | 30 | 0 | 82 |
| | 100 | 90 | 75 |
| c116 | 30 | 6 | 88 |
| | 100 | 87 | 91 |
| Ref 2: | 30 | 0 | 83 |
| 18-Hydroxy- | 100 | 79 | 72 |
| octadec-2-en-2- | | | |
| fluoro-oic acid | | | |
| c46 | 30 | 0 | 77 |
| | 100 | 75 | 68 |
| c30 | 10 | 42 | 77 |
| | 30 | 71 | 76 |
| c15 | 30 | 2 | 76 |
| | 100 | 73 | 74 |
| Ref3: | 30 | 0 | 92 |
| (E)-14- | 100 | 11 | 103 |
| tetrahydropyranyloxy- | | | |
| tetradec- | | | |
| 2-enoic acid | | | |
| Ref 4: | 30 | 0 | 114 |
| 14-Hydroxy- | 100 | 0 | 95 |
| tetraadec-2-en- | | | |
| 2-fluoro-oic | | | |
| acid | | | |

The inhibition of melanogenesis by the claimed compounds is demonstrated at the cellular level. Their depigmenting properties are superior to those of the reference compounds, in particular those of the family of unsaturated α,β hydroxy acids (ref 1, ref 2, ref 3, ref 4).

### Example 29

### Inhibition of Leukocyte Elastase

The elastases are a sub-family of serine proteases responsible for the degradation of elastin. The numerous natural substrates of this enzyme include, in addition to elastin, the proteoglycans of cartilage, fibronectin and the type I, II, III and IV collagens. At the cutaneous level, inhibition of elastase can combat the effects of ageing, whether or not photo-induced, and limit the appearance of wrinkles and stretch marks.
Biological model: human leukocyte elastase
Conditions:
   Control (n=6)
   Reference: AAPV (N-methoxysuccinyl-Ala-Ala-Pro-Val-chloromethyl ketone) 100 μM (n=6)
   compounds tested (n=2)
Incubation: 1 hour
Assay: The activity of human leukocyte elastase was evaluated using the EnzChek® Elastase assay kit.
   The measurement of activity is based on the use of a fluorescent substrate (DQTM Elastin) whose fluorescence is “quenched” through the presence of “quencher” groups at the level of the lytic sites. After action of the enzyme, the “quencher” groups are released and the fluorescence emitted is proportional to the activity of the enzyme.
   The results are expressed in percentage inhibition of the enzyme activity. They are presented in the following table:

| Treatment | | Normalized data | |
|---|---|---|---|
| | Concentration | | Standard deviation |
| Compound tested | (μM) | Inhibition (%) | (%) |
| Control | — | 0 | 5 |
| AAPV | 100 | 95 | 2 |
| DMSO | 0.3% | 0 | 2 |
| c129 | 30 | 57 | 2 |
| c128 | 30 | 75 | 1 |

### Example 30

### Proliferation of Aged Human Dermal Fibroblasts

In addition to a decrease in the production and an increase in the degradation of the extracellular matrix, skin ageing is accompanied by a decrease in proliferative capacity of the fibroblasts.

Thus, stimulation of the proliferation of the aged fibroblasts makes possible a partial reversal of the deleterious effects of ageing.
Biological model: Human dermal fibroblasts aged according to the Hayflick model (fibroblasts obtained by successive subculture) (P17NHDF)
Culture conditions: 37° C., 5% CO₂
Culture medium: DMEM/fetal calf serum (FCS) 10%
Conditions:
   Control (n=6)
   Control normal fibroblasts, not aged (P7 NHDF) (n=2)
   Reference: EGF (Epidermal Growth Factor) at 10 ng/mL (n=2)
   compounds tested (n=2)
Incubation: 72 hours
Assay: The effects on proliferation were evaluated by measuring the incorporation of [³H]-thymidine in fibroblasts aged according to the Hayflick model. The Hayflick model consists of applying successive subcultures in order to induce an “aged” phenotype.
   The results are expressed in percentage stimulation of the incorporation of [³H]-thymidine. They are presented in the following table:

| Treatment | | Normalized data | |
|---|---|---|---|
| Compounds | Concentrations | | standard deviation |
| tested | (μM) | Stimulation (%) | (%) |
| Control P17 | — | 0 | 14 |
| Control P7 | — | 308 | 86 |
| EGF | 10 ng/ml | 89 | 5 |
| c85 | 10 | 22 | 1 |
| c7 | 10 | 63 | 13 |
| c42 | 3 | 48 | 64 |

### Example 31

### Proliferation and/or Migration of Fibroblasts

The phases of migration and proliferation of the cells are major phases in healing, which occur after the inflammation phase. They are necessary for recolonization of the wound.

An increase in migration and proliferation of the cells allows an improvement in healing.
Biological model: Normal human dermal fibroblasts (NHDF)
Culture conditions: 37° C., 5% CO₂
Culture medium: DMEM
Conditions:
   Control (NHDF in DMEM test medium 0% FCS) (n=6)
   Reference: FCS (Fetal Calf Serum) at 10% (n=2)
   compounds tested (n=2)
Incubation: 72 hours
Assay: Normal human fibroblasts were seeded on 96-well plates suitable for the migration studies. In these plates, the substrates were pretreated with a solution of collagen and a mask was placed at the centre of each well, preventing adhesion of the cells in this zone and thus forming an artificial wound. After labelling the cells with calcein, the masks were removed and then the cells were treated with the compounds or the reference.
   The migration of the cells was monitored microscopically for 72 hours, taking photographs at 0 h, 24 h, 48 h and 72 h.
   The results are expressed in percentage coverage and compared with the untreated control. They are presented in the following table:

| | Concentration | % control | standard |
|---|---|---|---|
| Treatment | (μM) | 24 h | deviation (%) |
| Control | — | 100 | 3 |
| FCS | 10 ng/ml | 154 | 39 |
| DMSO | 0.1% | 112 | 13 |
| c132 | 10 | 131 | 8 |
| c90 | 1 | 132 | 20 |
| c27 | 10 | 131 | 14 |
| c42 | 3 | 130 | 16 |

### Example 32

### Proliferation and/or Migration of Keratinocytes

The phases of migration and proliferation of cells are major phases of healing that occur after the inflammation phase and are necessary for recolonization of the wound.
An increase in the migration and proliferation of cells allows an improvement in healing.
Biological model: Normal human epidermal keratinocytes (NHEK)
Culture conditions: 37° C., 5% CO₂
Culture medium: Keratinocyte-SFM-PE-EGF (keratinocyte culture medium—serum free medium (SFM) without pituitary extract (PE) and without Epidermal Growth Factor (EGF)
Conditions:
   Control (n=6)
   Reference: EGF (Epidermal Growth Factor) at 10 ng/ml (n=2)
   compounds tested (n=2)
Incubation: 72 hours
Assay: Normal human keratinocytes were seeded on 96-well plates suitable for the migration studies. In these plates, the substrates were pretreated with a solution of collagen and a mask was placed at the centre of each well, preventing adhesion of the cells in this zone and thus forming an artificial wound. After labelling the cells with calcein, the masks were removed and then the cells were treated with the compounds or the reference.
   The migration of the cells was monitored microscopically for 72 hours, taking photographs at 0 h, 24 h, 48 h and 72 h.
   The results are expressed in percentage coverage and compared with the untreated control. They are presented in the following table:

| | Concentration | % control | standard |
|---|---|---|---|
| Treatment | (μM) | 48 h | deviation (%) |
| Control | — | 100 | 9 |
| EGF | 10 ng/mL | 161 | 5 |
| DMSO | 0.1% | 76 | 5 |
| c79 | 1 | 141 | 3 |
| c85 | 10 | 176 | 2 |
| c132 | 10 | 138 | 5 |
| c40 | 30 | 144 | 12 |
| c17 | 1 | 141 | 9 |
| c90 | 1 | 130 | 3 |
| c36 | 3 | 154 | 7 |
| c45 | 3 | 145 | 6 |
| c42 | 3 | 141 | 15 |
| c53 | 3 | 137 | 17 |

1. Compounds represented by general formula I shown below 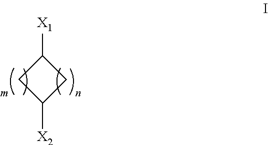
in which
m=1, 2, 3 and n=0, 1
provided that m+n is different from 4,
X₁ and X₂ can be trans or cis relative to one another and represent, independently of one another, a group selected from 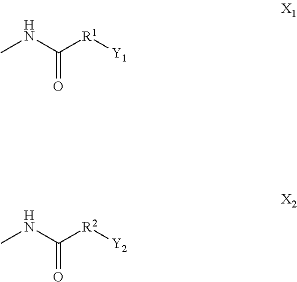
in which [symbol]
Y₁ and Y₂ represent, independently of one another,
—H,
—OH,
—OH optionally coupled to a glycoside compound, which can be an α- or β-furanose or an α- or β-pyranose,
—ORₐ,
—OCOCH₃,
—OSi(Rₐ)₃.
—OSitBdPh of formula 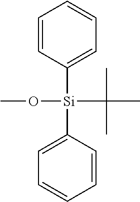 [SMILES: CO[Si](c1ccccc1)(c1ccccc1)C(C)(C)C]
—OSitBdM of formula 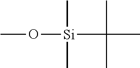
—COOH,
—COOR_{b},
—NH₂,
—NR_{c}R_{d},
—NHCORₑ,
—NHCOOR_{f},
the —OTHP group of formula 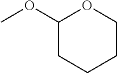
a group derived from ethylene glycol of formula, 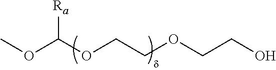
in which δ varies from 1 to 12,
a group derived from propylene glycol of formula, 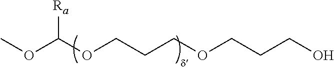
in which δ′ varies from 1 to 5,
an —O—CH(R_{z})—O-Q group, in which R_{z}, represents an alkyl or aralkyl group comprising from 1 to 30 carbon atoms, which can, but does not necessarily, contain one or more ether functions and optionally a terminal hydroxyl,
Rₐ, R_{b}, R_{c}, R_{d} represent linear or branched alkyl groups comprising from 1 to 4 carbon atoms, optionally substituted by one or more halogen atoms, or carbon chains interrupted by oxygen or sulphur atoms, benzyl groups optionally substituted by a halogen atom, a hydroxy group, an alkoxy group having from 1 to 8 carbon atoms,
Rₑ represents a linear or branched alkyl group containing from 1 to 4 carbon atoms, a phthalimido group (in this case the NH is replaced by N), a benzyl group optionally substituted by a halogen atom, a hydroxy group, an alkoxy group and in particular substituted by the methoxy group in the para position,
R_{f} represents a linear or branched alkyl group comprising from 1 to 4 carbon atoms, optionally substituted by one or more halogen atoms, or a carbon chain interrupted by oxygen or sulphur atoms, a phenyl group, a benzyl group optionally substituted by a halogen atom, a hydroxy group, an alkoxy group and in particular substituted by the methoxy group in the para position,
the phosphonate group of formula 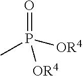
in which
R⁴ represents a linear or branched alkyl group, comprising from 1 to 6 carbon atoms, in particular methyl, ethyl, isopropyl, tert-butyl, (OR⁴)₂ optionally forming a ring between the two oxygen atoms, the (OR⁴)₂ groups in particular originating from diols such as ethane-1,2-diol, propane-1,3-diol, 2,2-dimethylpropane-1,3-diol, 2,3-dimethylbutane-2,3-diol (pinacol), 2-methylbutane-2,3-diol, 1,2-diphenylethane-1,2-diol, 2-methylpentane-2,4-diol, 1,2-dihydroxybenzene (catechol), 2,2′-azanediyldiethanol, 2,2′-(butylazanediyl)diethanol, 2,3-dihydroxysuccinic acid (tartaric acid) and its esters, or (OR⁴)₂ originates in particular from diacids such as 2,2′-(methylazanediyl)diacetic acid (mida), [symbol]
R¹ and R² represent, independently of one another, linear or branched chains having from 1 to 30 carbon atoms, R¹ and R² being saturated or unsaturated, unsubstituted or substituted by a halogen atom,
and in the case of an unsaturation, the C═C double bond optionally being substituted by a fluorine, chlorine, or bromine atom or by a —CF₃ group,
and in the case when R¹ and R² only comprise a single carbon, they are selected from the groups of Formula “—CHV—”, in which V represents —H, —F, —Cl or —Br, Y₁ and Y₂ then being equal to the phosphonate group —P(O)(OR⁴)₂, R⁴ having the meaning given above,
and in particular in which X₁ and X₂ are identical or different and correspond to Formula I_{A} or I_{B} 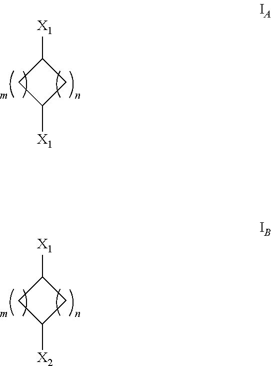
in which
X₁, X₂, m and n have the meanings as previously defined.

2. Compounds according to claim 1, of Formula II 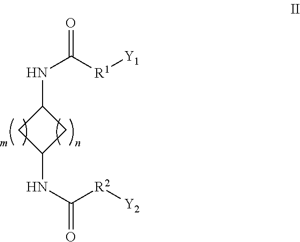
in which
R¹, R², Y₁, Y₂, m and n have the meanings as previously defined,
R¹, R² are identical or different,
Y₁ and Y₂ are identical or different,
m=1, 2, 3,
n=0, 1,
provided that m+n is different from 4,
and in particular of Formula II_{cis} 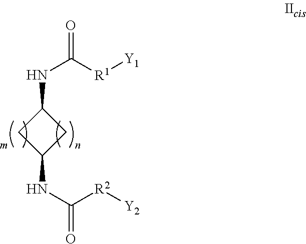
in which
R¹, R², Y₁, Y₂, m and n have the meanings as previously defined,
R¹, R² are identical or different,
Y₁ and Y₂ are identical or different,
m=1, 2, 3,
n=0, 1,
provided that m+n is different from 4,
and in particular of Formula II_{B cis} 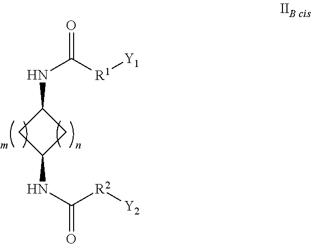
in which
R¹, R², Y₁, Y₂, m and n have the meanings as previously defined,
provided that if R¹ and R² are identical, then Y₁ and Y₂ are different,
provided that if R¹ and R² are different, then Y₁ and Y₂ are identical or different,
m=1, 2, 3,
n=0, 1,
provided that m+n is different from 4,
and in particular of Formula IIₜᵣₐₙₛ 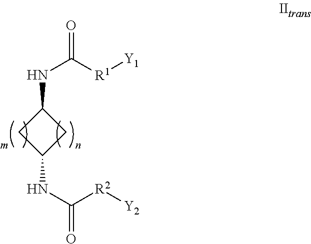
in which
R¹, R², Y₁, Y₂, m and n have the meanings as previously defined,
R¹, R² are identical or different,
Y₁ and Y₂ are identical or different,
m=1, 2, 3,
n=0, 1,
provided that m+n is different from 4,
and in particular of Formula II_{A trans} 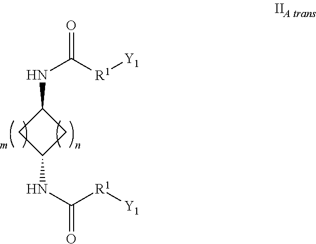
in which
R¹, Y₁, m and n have the meanings as previously defined,
m=1, 2, 3,
n=0, 1,
provided that m+n is different from 4,
and in particular of Formula II_{B trans} 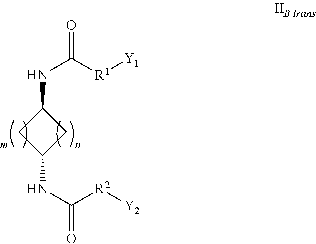
in which
R¹, R², Y₁, Y₂, m and n have the meanings as previously defined,
provided that if R¹ and R² are identical, then Y₁ and Y₂ are different,
provided that if R¹ and R² are different, then Y₁ and Y₂ are identical or different,
m=1, 2, 3,
n=0, 1,
provided that m+n is different from 4.

3. Compounds according to claim 1, of Formula II_{A cis} 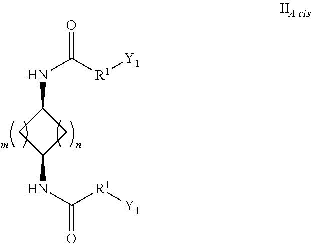
in which
R¹, Y₁, m and n have the meanings as previously defined,
m=1, 2, 3,
n=0, 1,
provided that m+n is different from 4.

4. Compounds according to claim 1, of Formula I in which n is equal to 0 and m is equal to 1, and corresponding to Formulae V_{A} or V_{B} 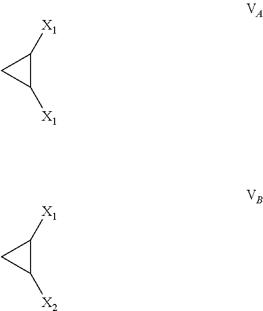
in which
X₁, X₂, m and n have the meanings as previously defined,
or of Formula I in which
n+m is equal to 2 and which correspond to general formula XXII 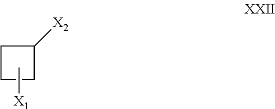
in which
X₁, X₂, n and m have the meanings as previously defined,
or of Formula I in which
n+m is equal to 2 and correspond to Formulae XXII_{A} or XXII_{B} 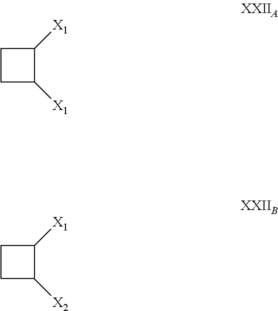
in which
X₁, X₂, m and n have the meanings as previously defined,
or of Formula I in which
n is equal to 1 and m is equal to 1,
and correspond to Formulae XXII_{F} and XXII_{G} shown below: 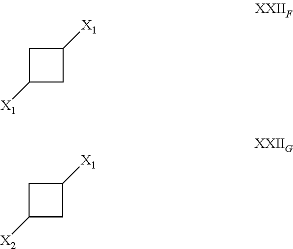
in which
X₁, X₂, m and n have the meanings as previously defined.

5. Compounds according to claim 1, of Formula I in which n+m is equal to 3, and which correspond to general formula VI 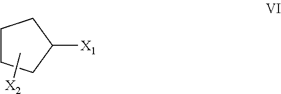
in which
X₁, X₂, n and m have the meanings as previously defined,
or in which n is equal to 0 and m is equal to 3 and correspond to Formulae VI_{A} and VI_{B} shown below: 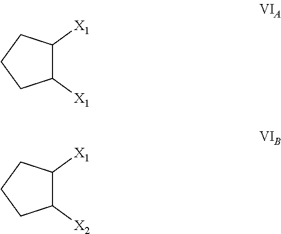
in which
X₁, X₂, m and n have the meanings as previously defined,
or in which n is equal to 1 and m is equal to 2,
and which correspond to Formulae VI_{F} and VI_{G} represented below 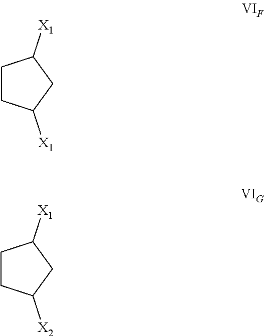
in which
X₁, X₂, m and n have the meanings as previously defined.

6. Compounds according to claim 5, of Formula VI_{F cis} 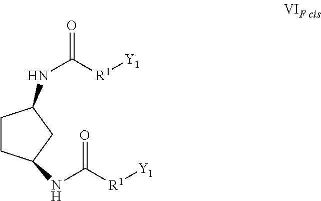
in which
R¹ and Y₁ have the meanings as previously defined,
or of Formula VI_{G cis} 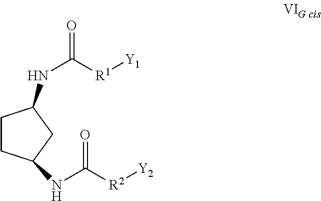
in which
R¹, R², Y₁, Y₂ have the meanings as previously defined,
provided that if R¹ and R² are identical, then Y₁ and Y₂ are different,
provided that if R¹ and R² are different, then Y₁ and Y₂ are identical or different,
m=1, 2, 3,
n=0, 1,
provided that m+n is different from 4,
or of Formula VI_{F trans} 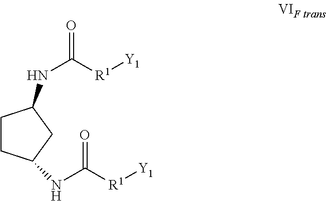
in which
R¹ and Y₁ have the meanings as previously defined,
or of Formula VI_{G trans} 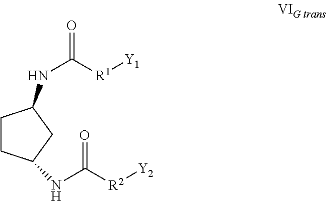
in which
R¹, R², Y₁, Y₂ have the meanings as previously defined,
provided that if R¹ and R² are identical, then Y₁ and Y₂ are different,
provided that if R¹ and R² are different, then Y₁ and Y₂ are identical or different,
m=1, 2, 3,
n=0, 1,
provided that m+n is different from 4.

7. Compounds of general formula I according to claim 1, in which X₁ and X₂ are shown below: 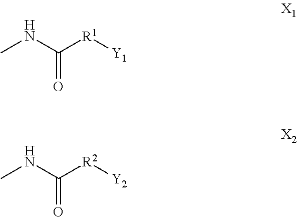
R¹ and R² representing, independently of one another, linear or branched chains, having from 1 to 30 carbon atoms,
the R¹—Y₁ and R²—Y₂ groups representing, independently of one another, one of the groups with the following formulae, where the amine radical can optionally be substituted, the terminal hydroxyl radical can optionally be coupled to a glycoside residue selected from the α- or β-furanoses and the α- or β-pyranoses, or coupled to a linear aliphatic chain comprising one or more oxygen atoms, of formulae shown below, 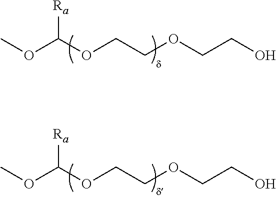
in which
δ varies from 1 to 12, δ′ varies from 1 to 5,
or a radical that can optionally be protected,
Rₐ representing a linear or branched alkyl group comprising from 1 to 4 carbon atoms, optionally substituted by one or more halogen atoms, 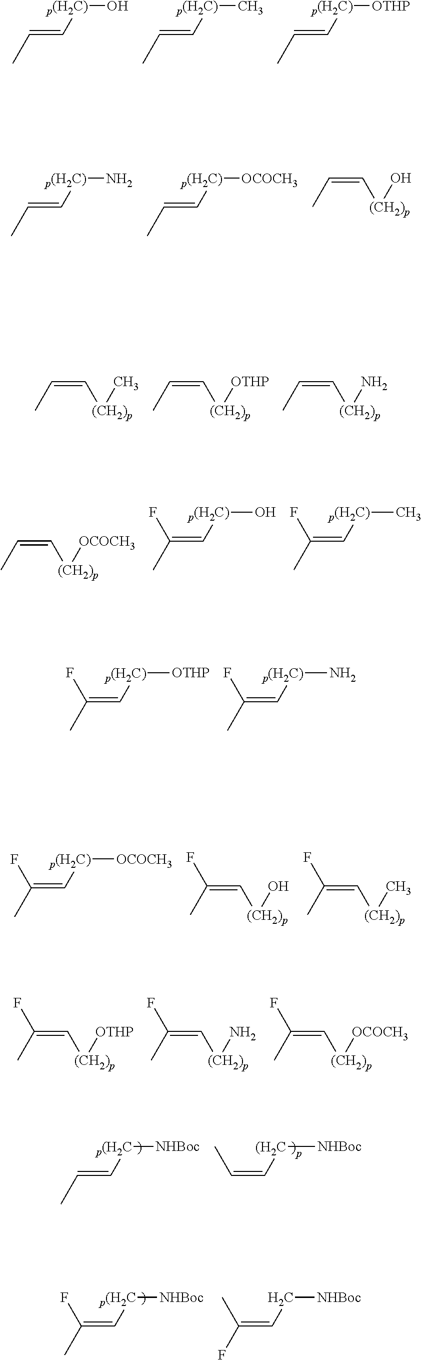 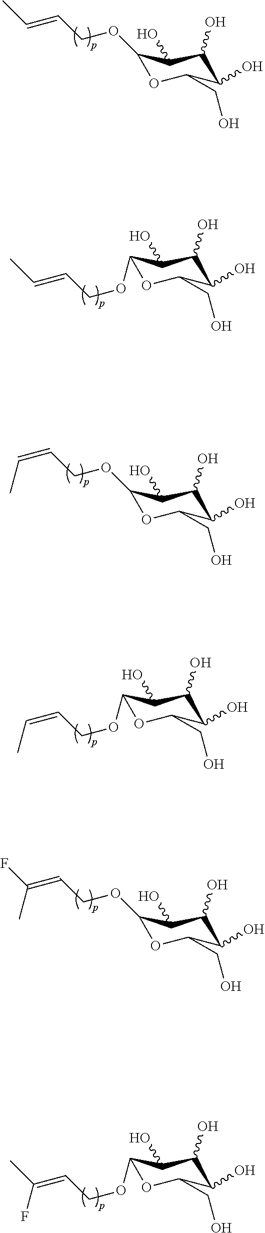 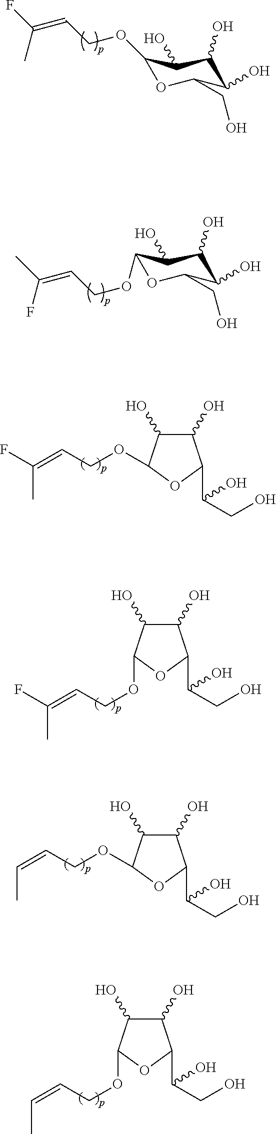 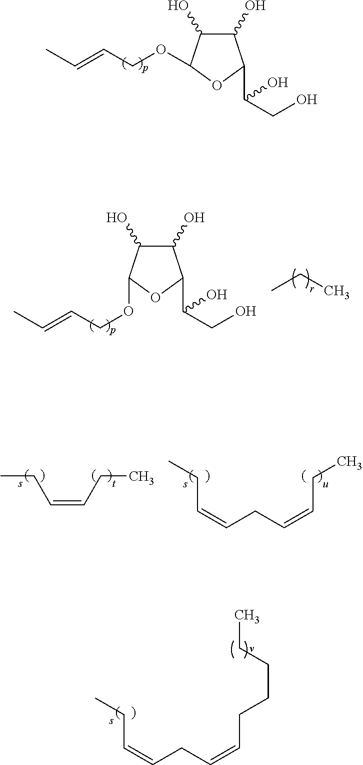
in which
p varies from 1 to 28,
r varies from 1 to 29,
s+t varies from 2 to 27,
s+u varies from 2 to 24,
s+v varies from 2 to 21.

8. Compounds according to claim 1, of general formula I, shown below:

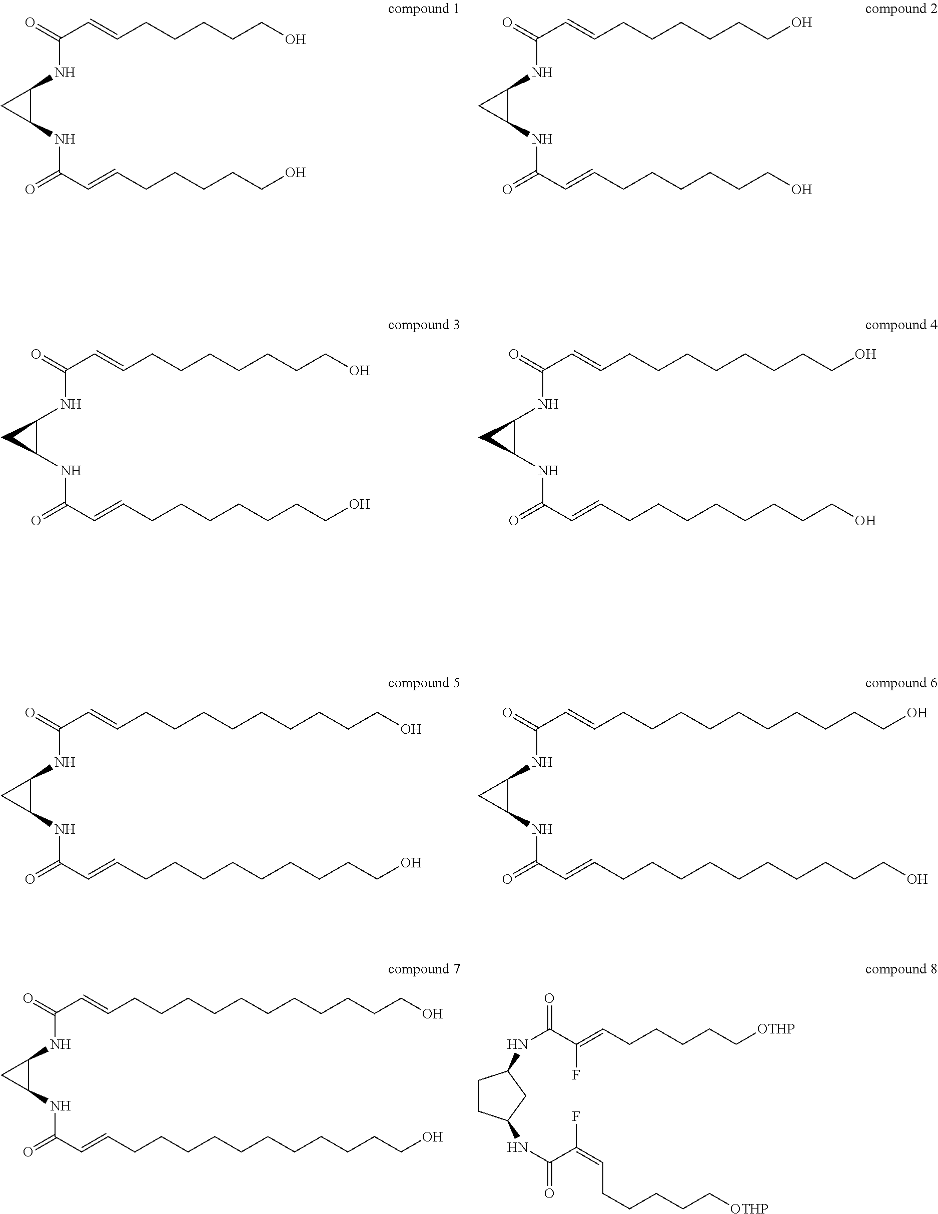

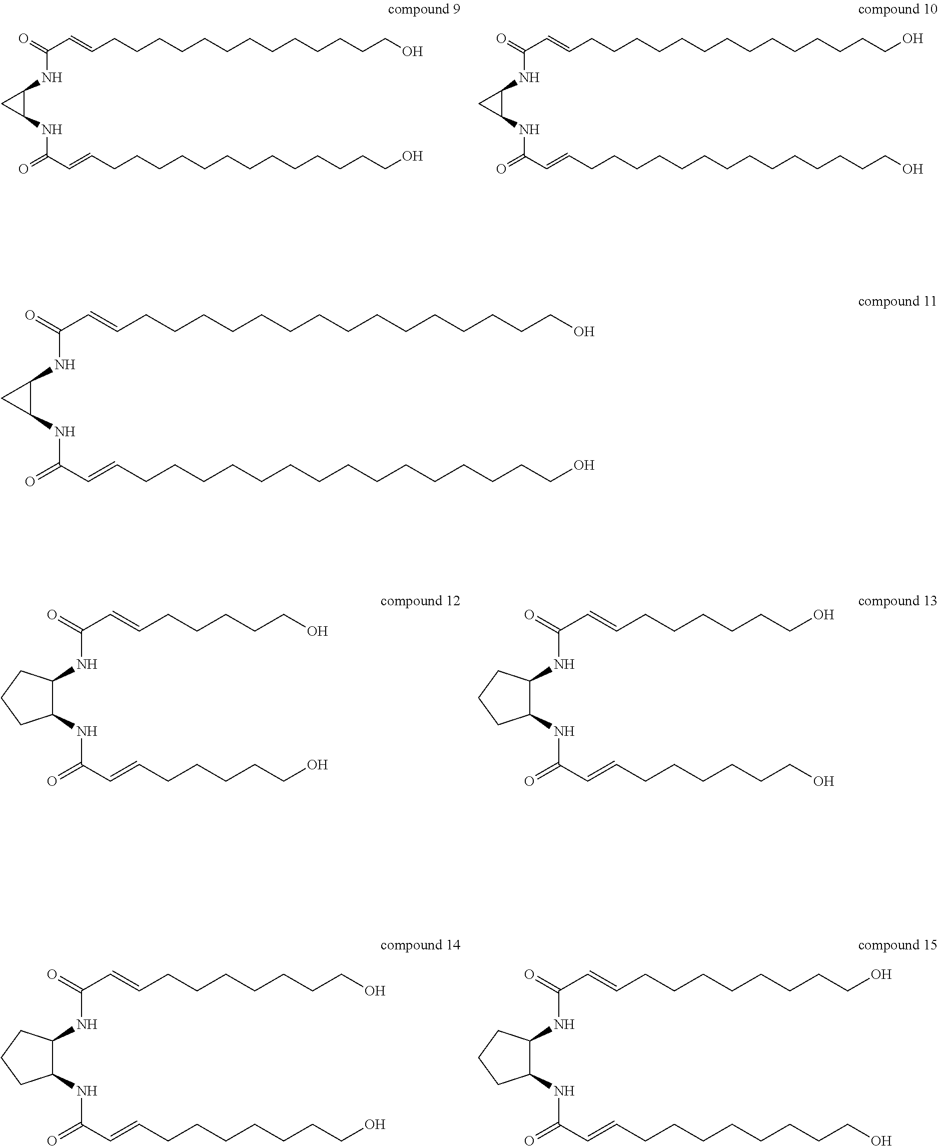

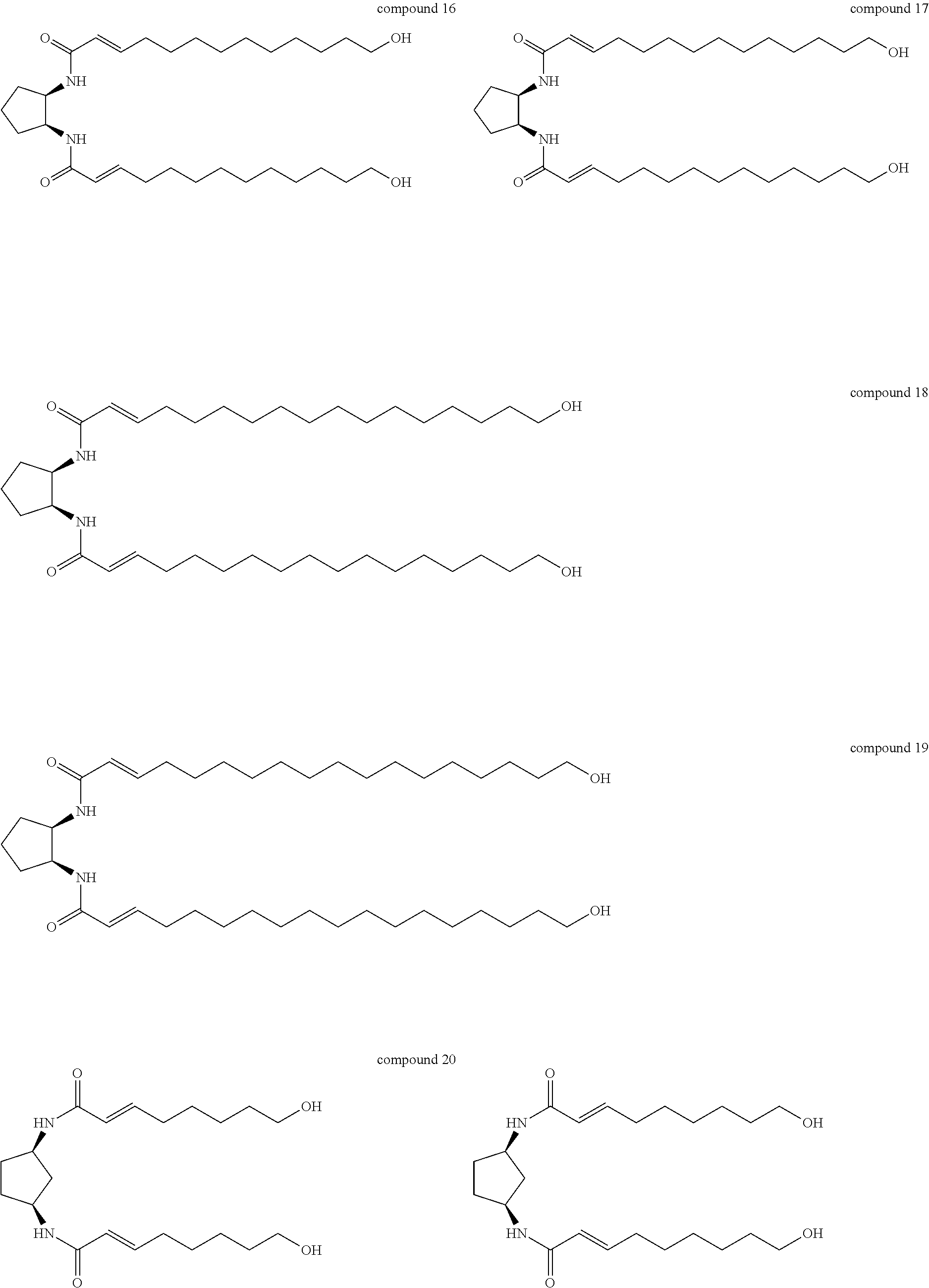

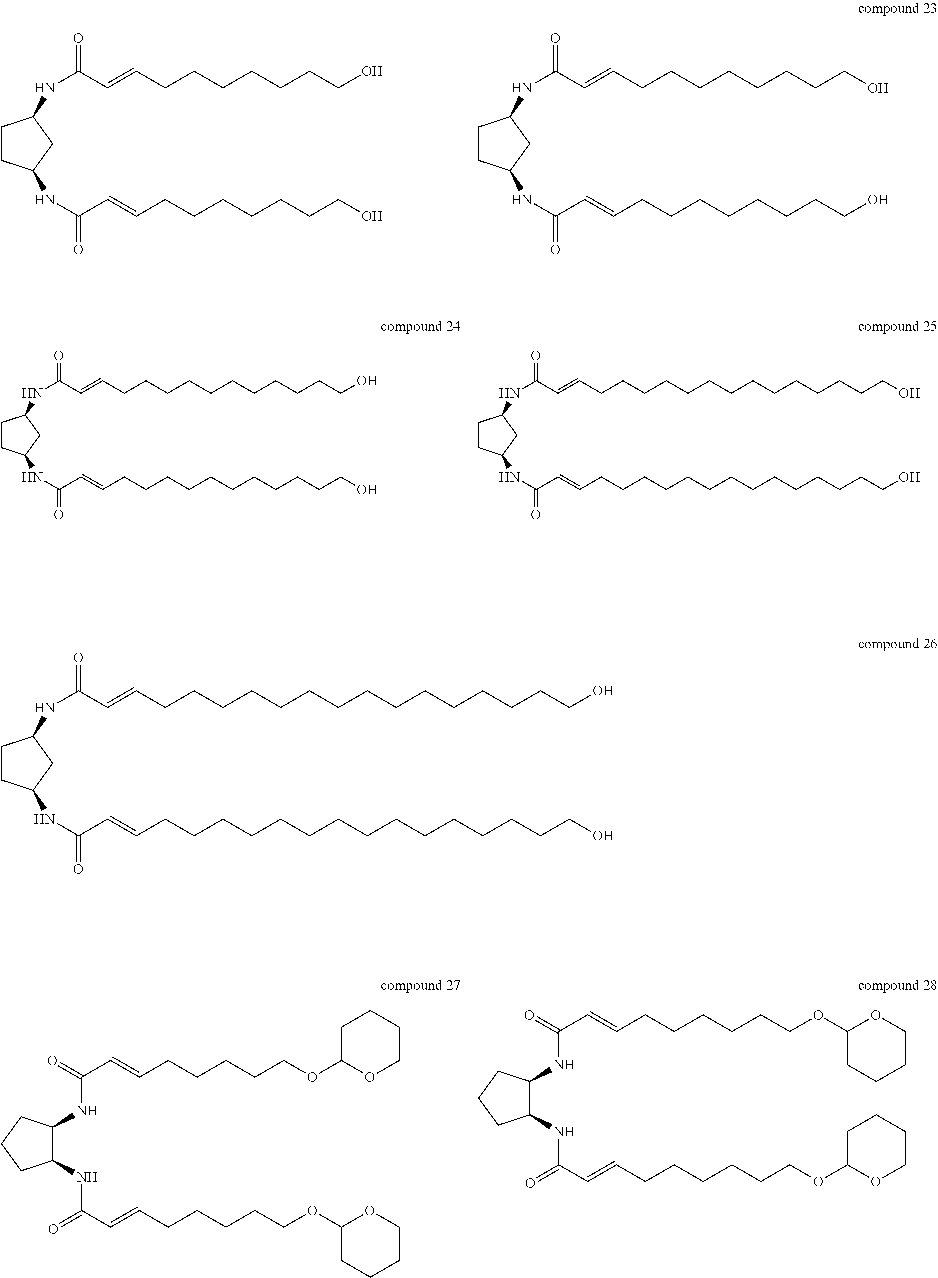

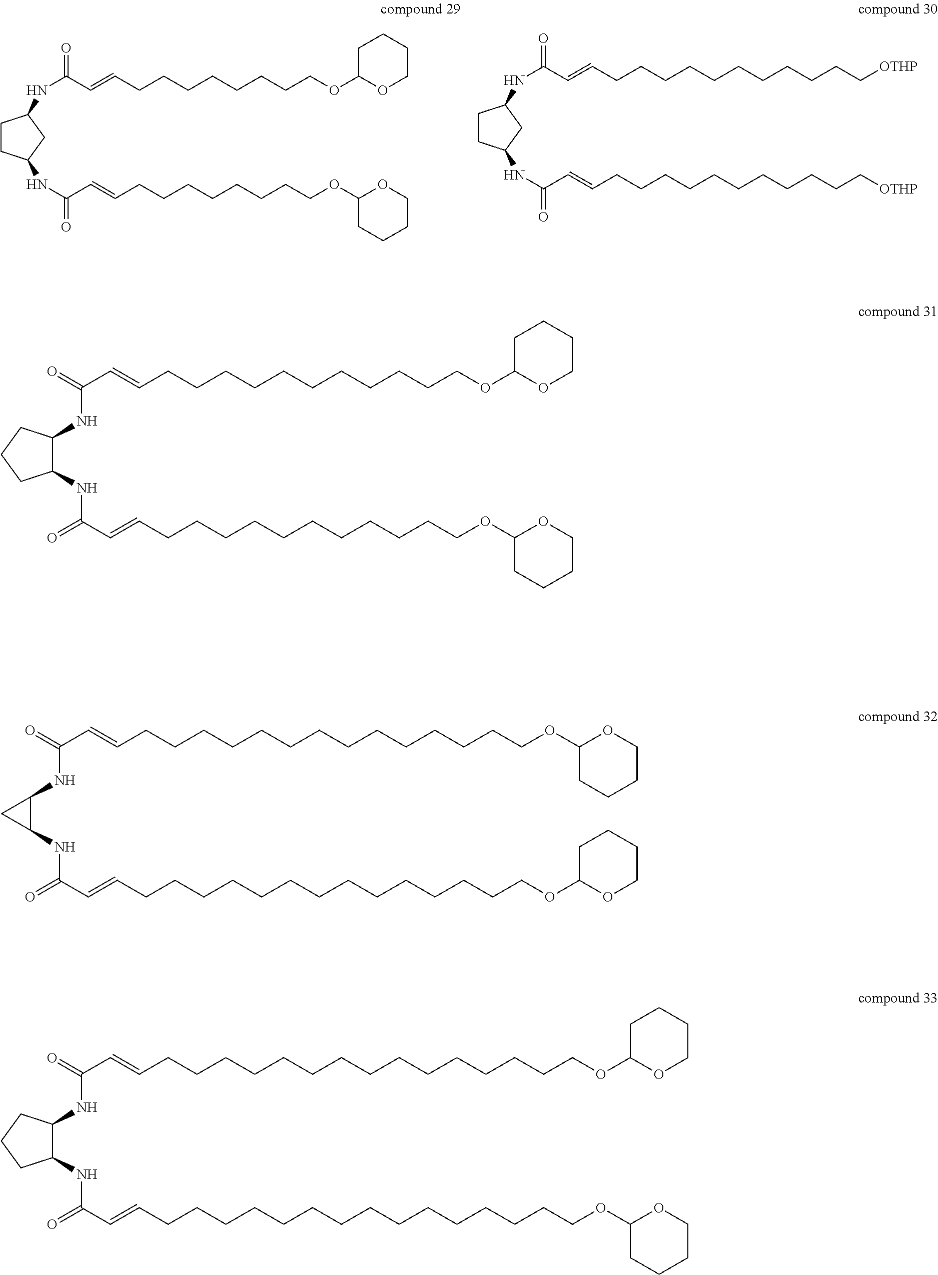

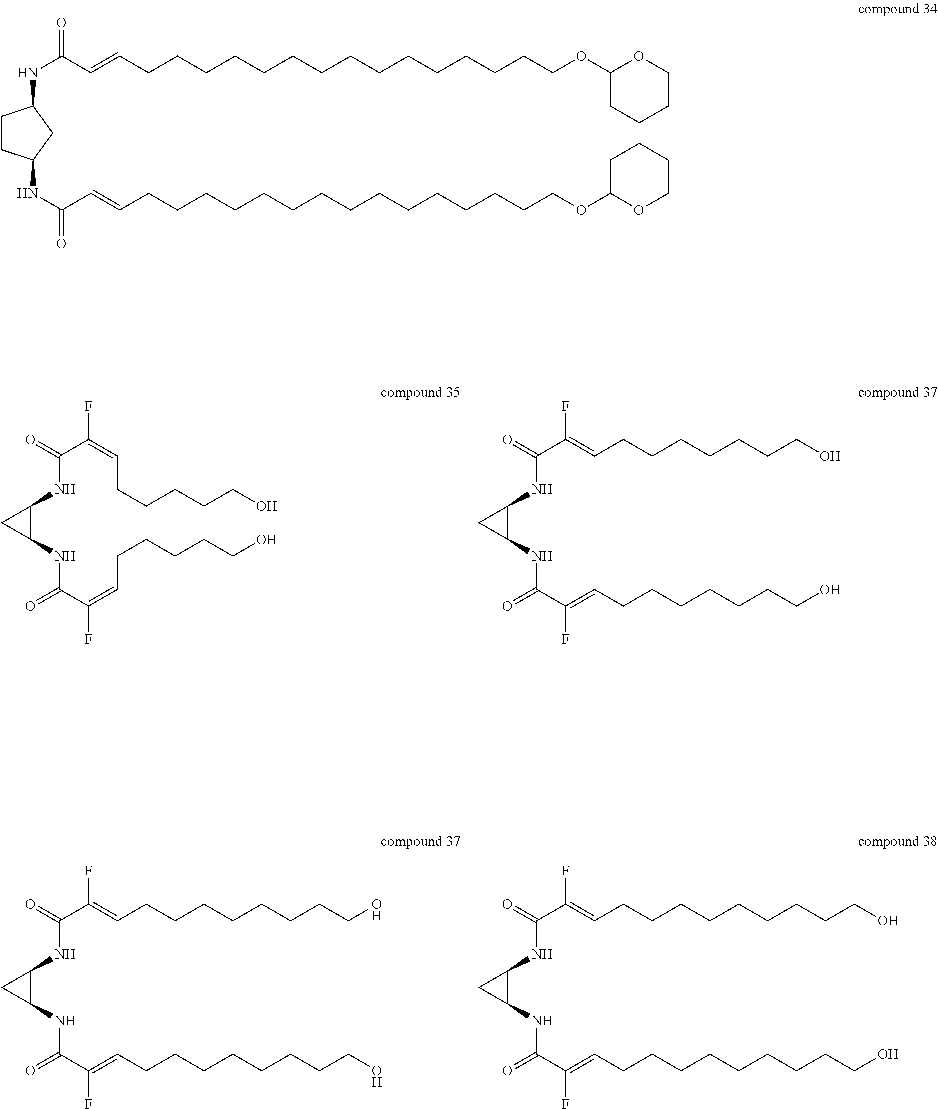

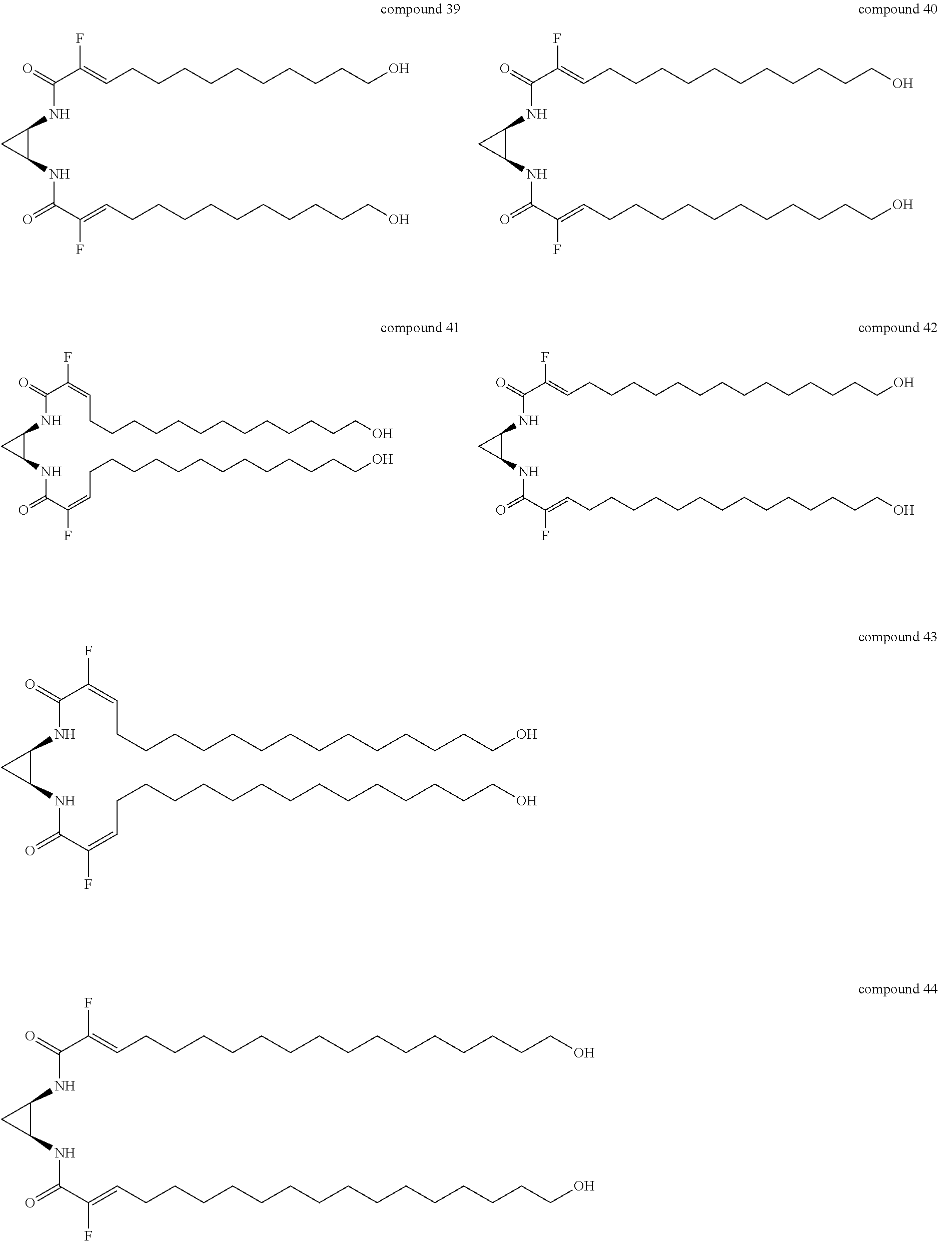

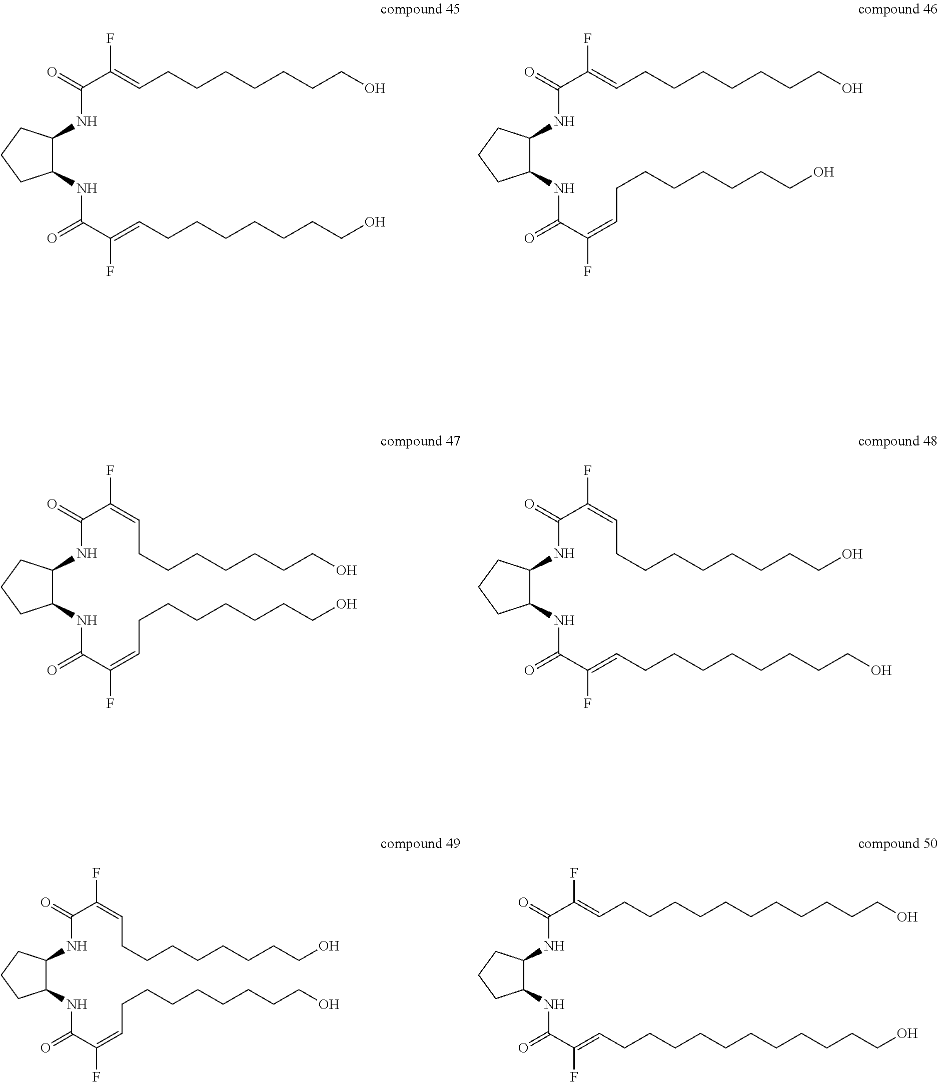

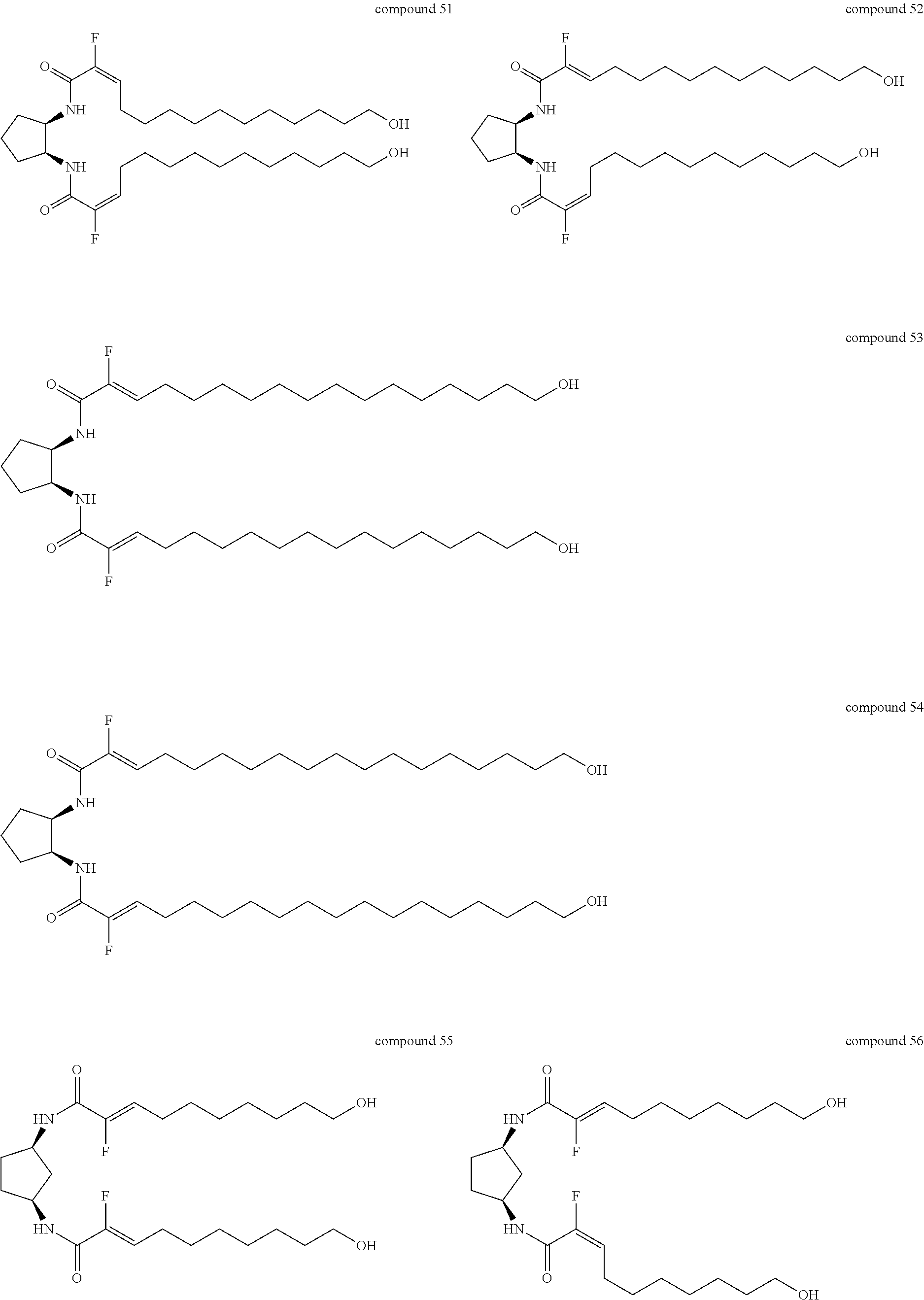

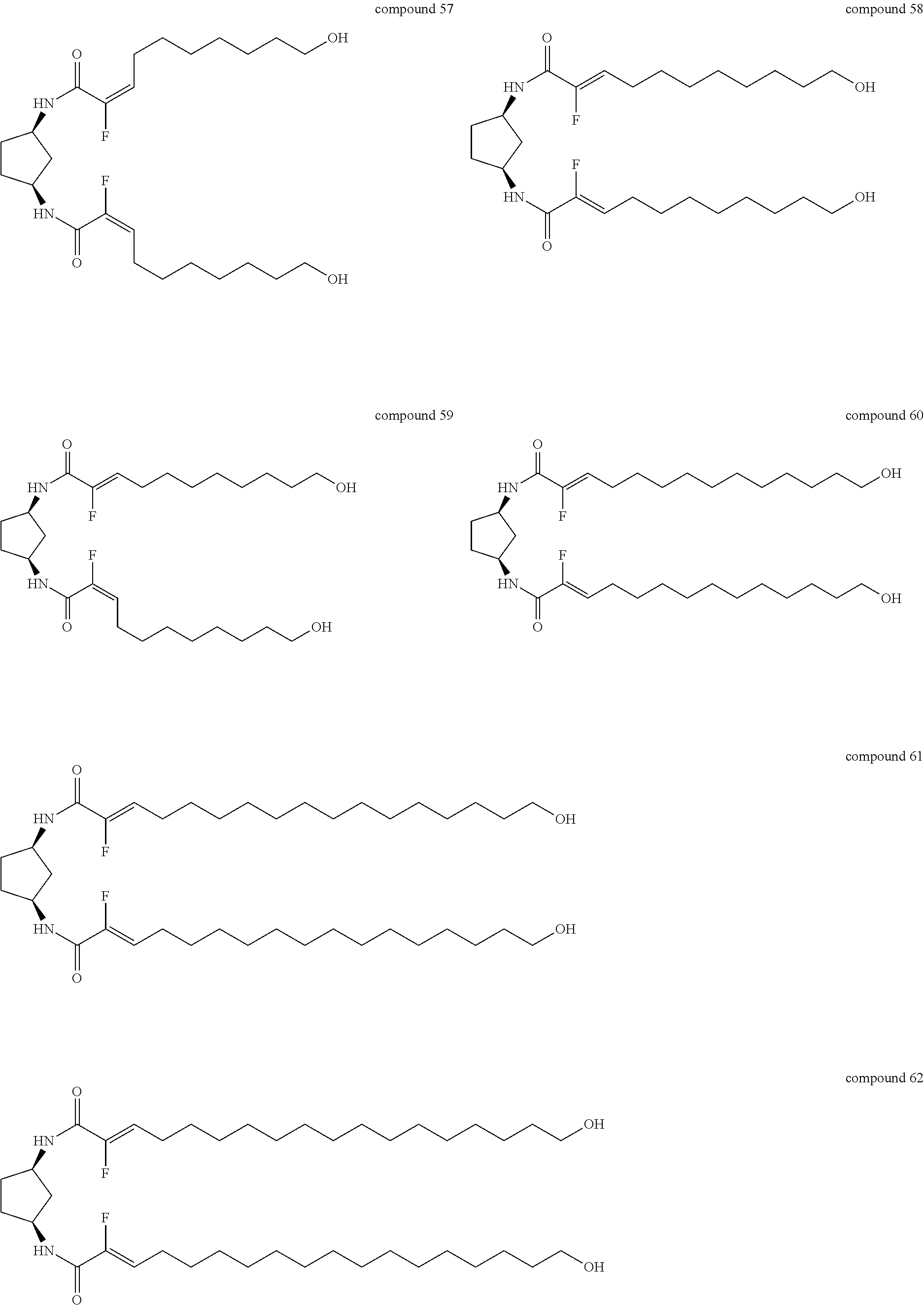

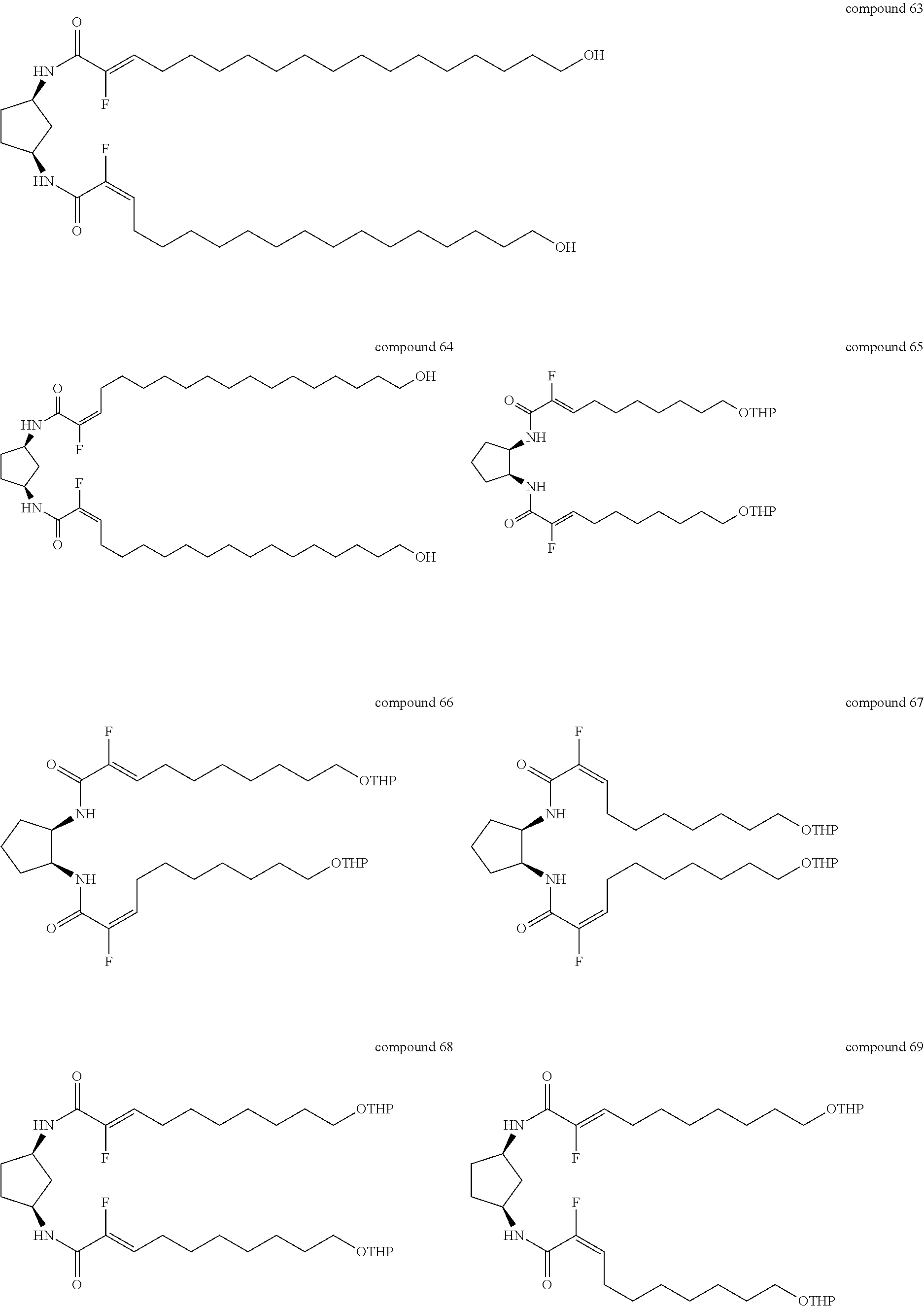

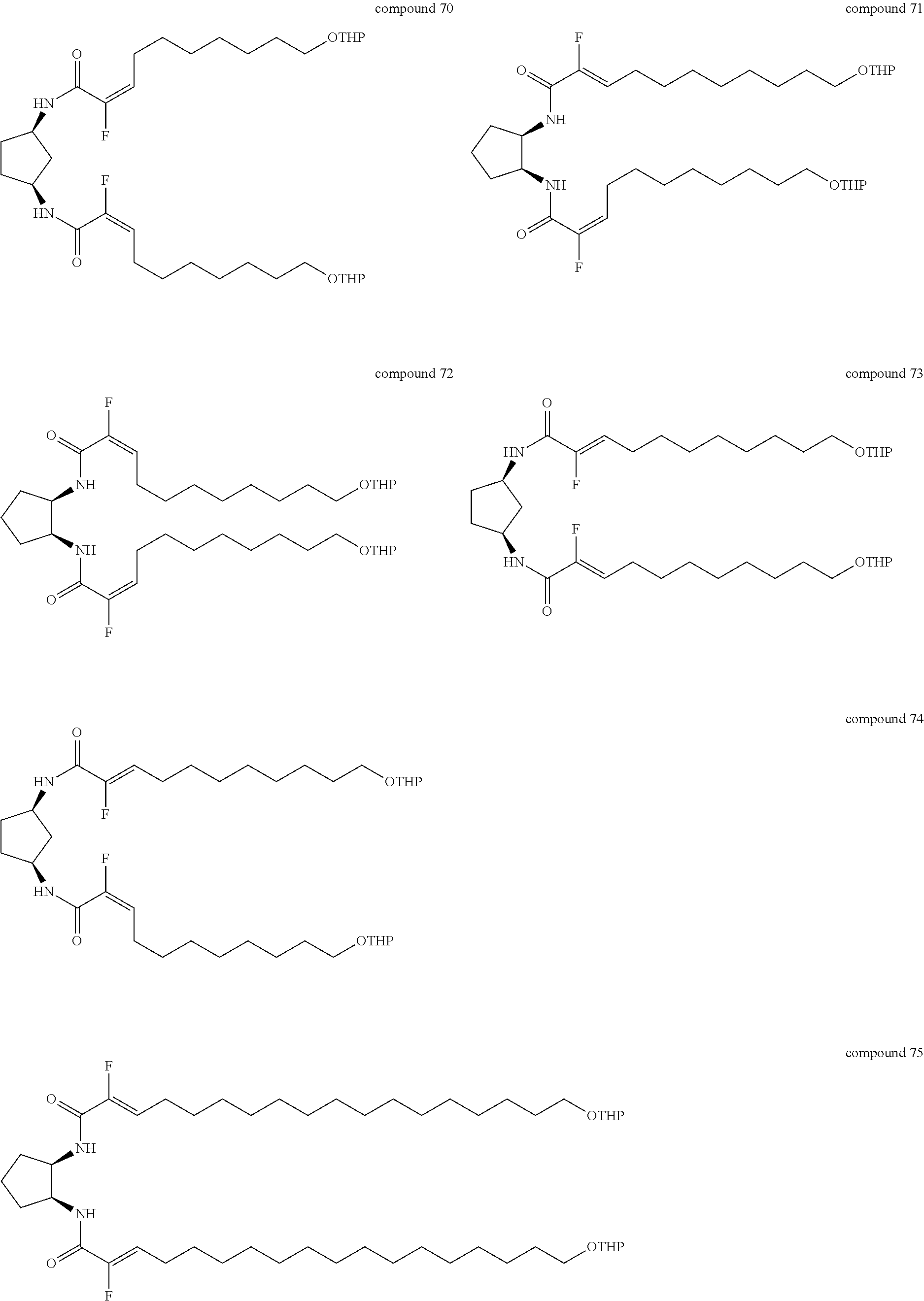

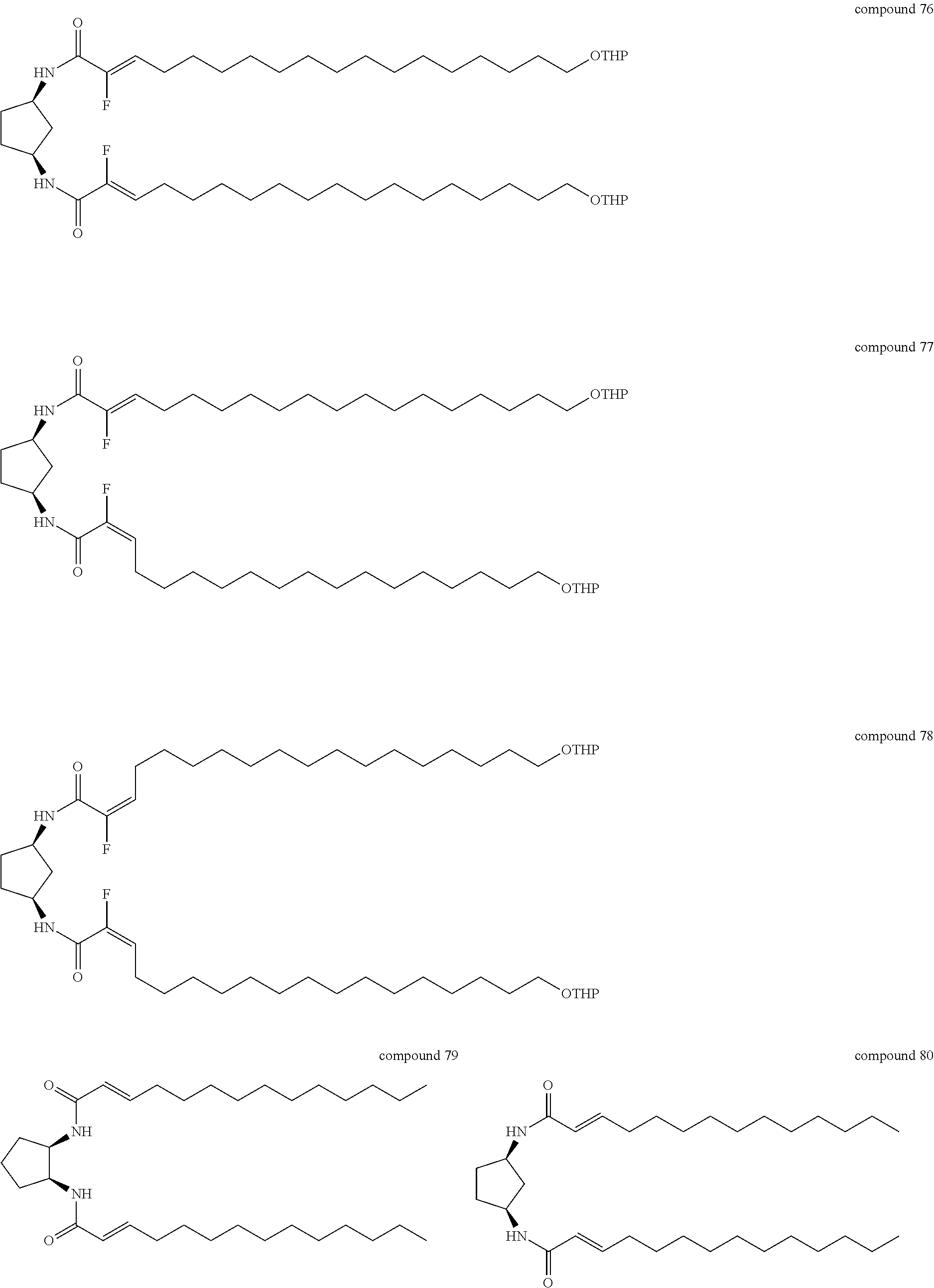

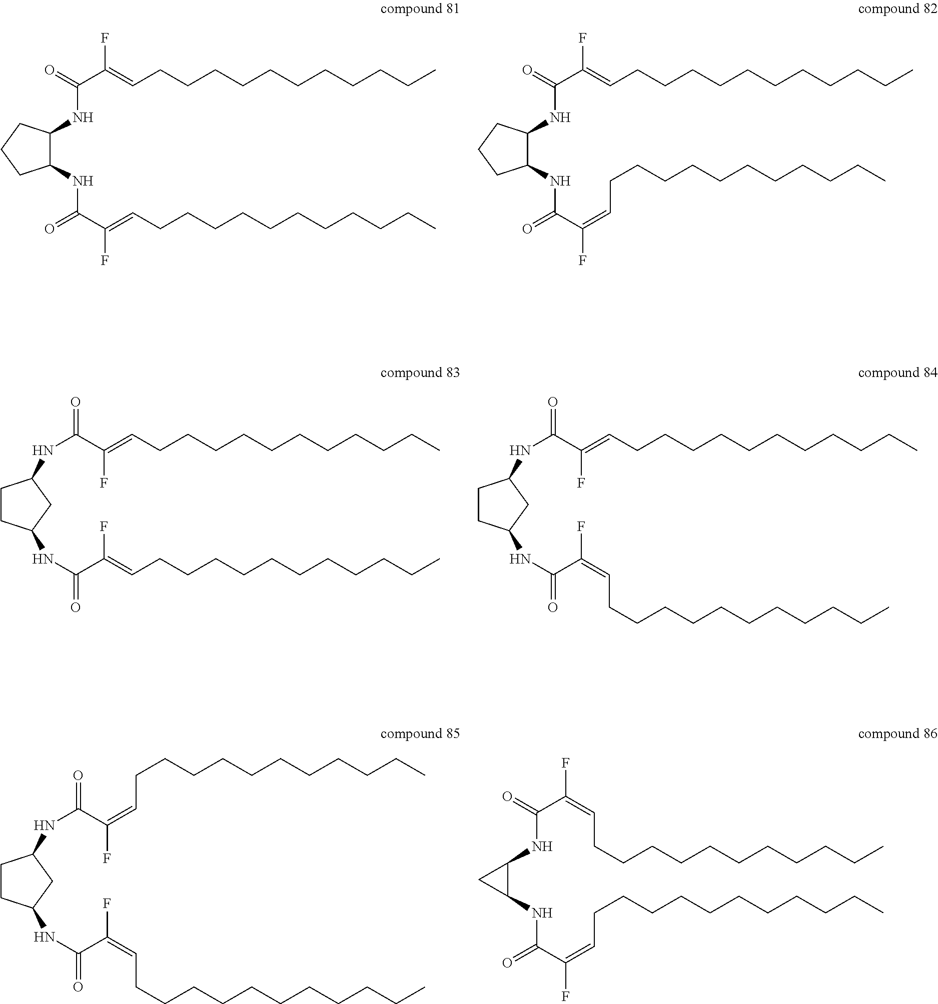

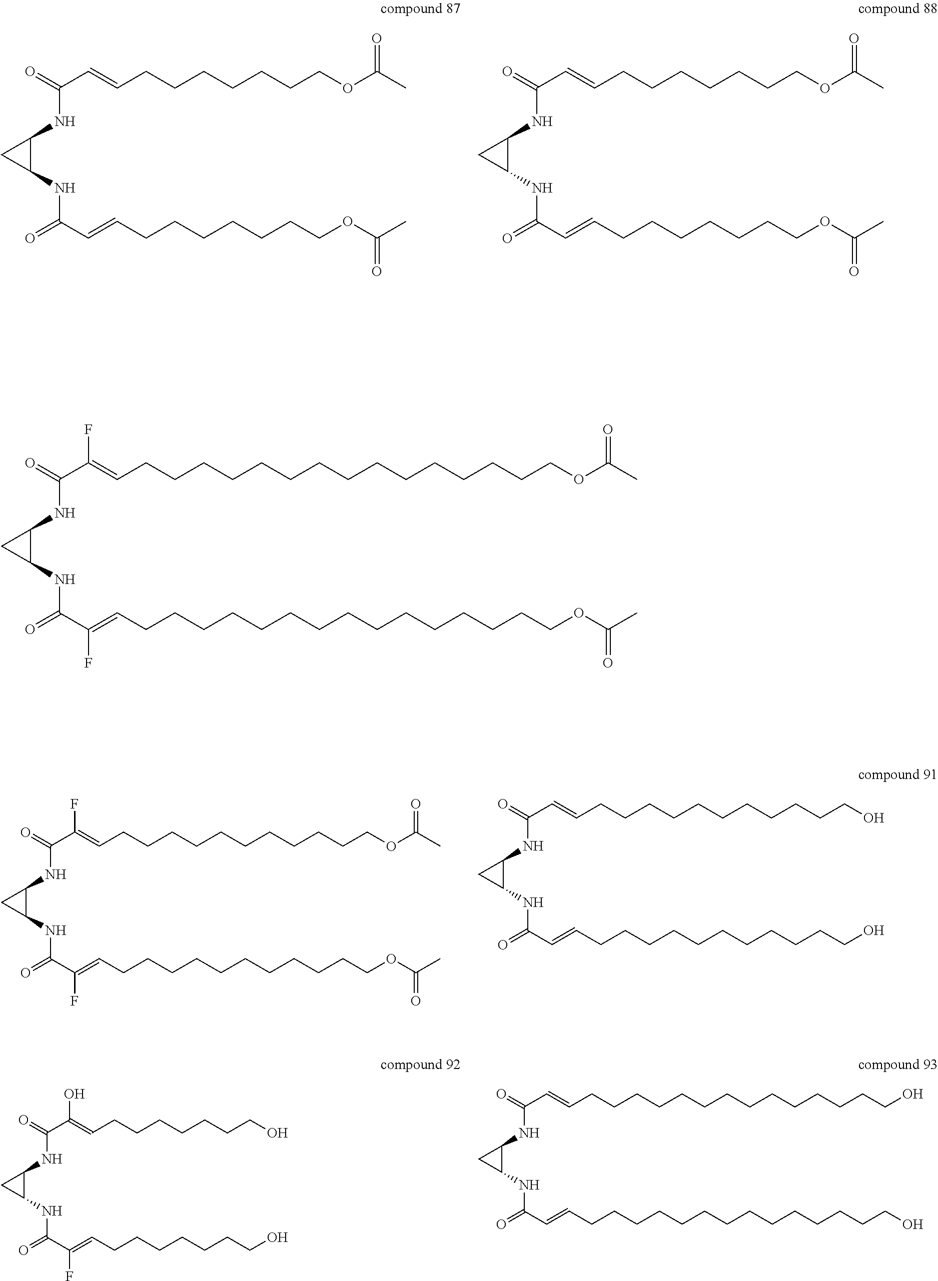

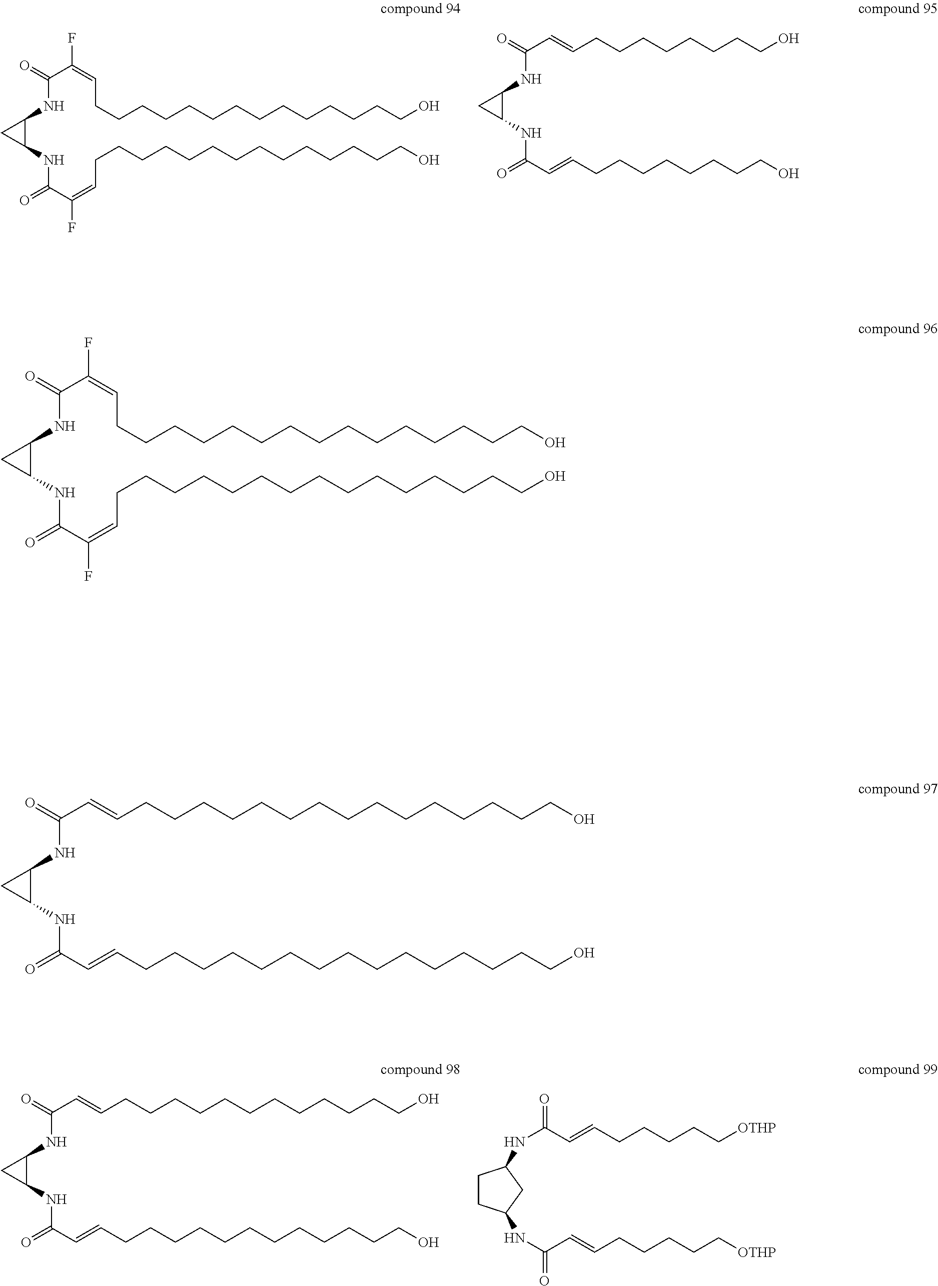

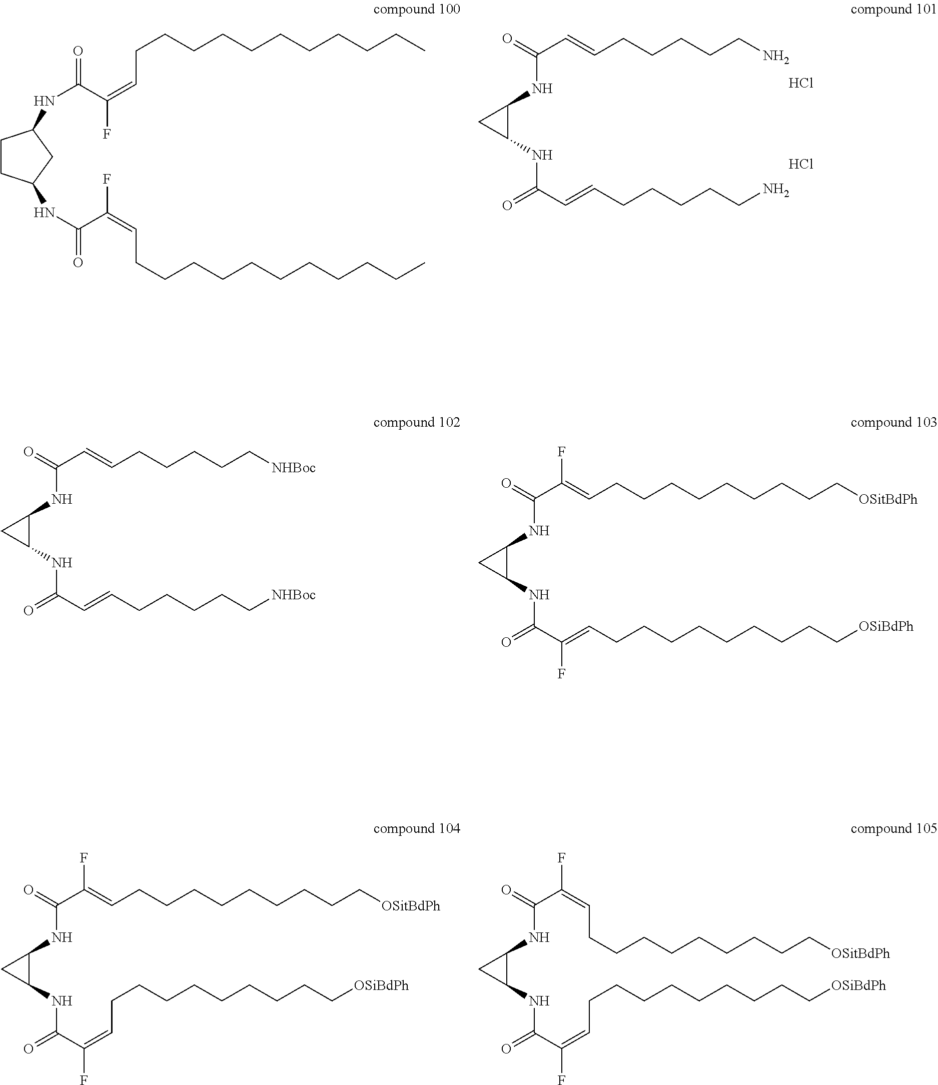

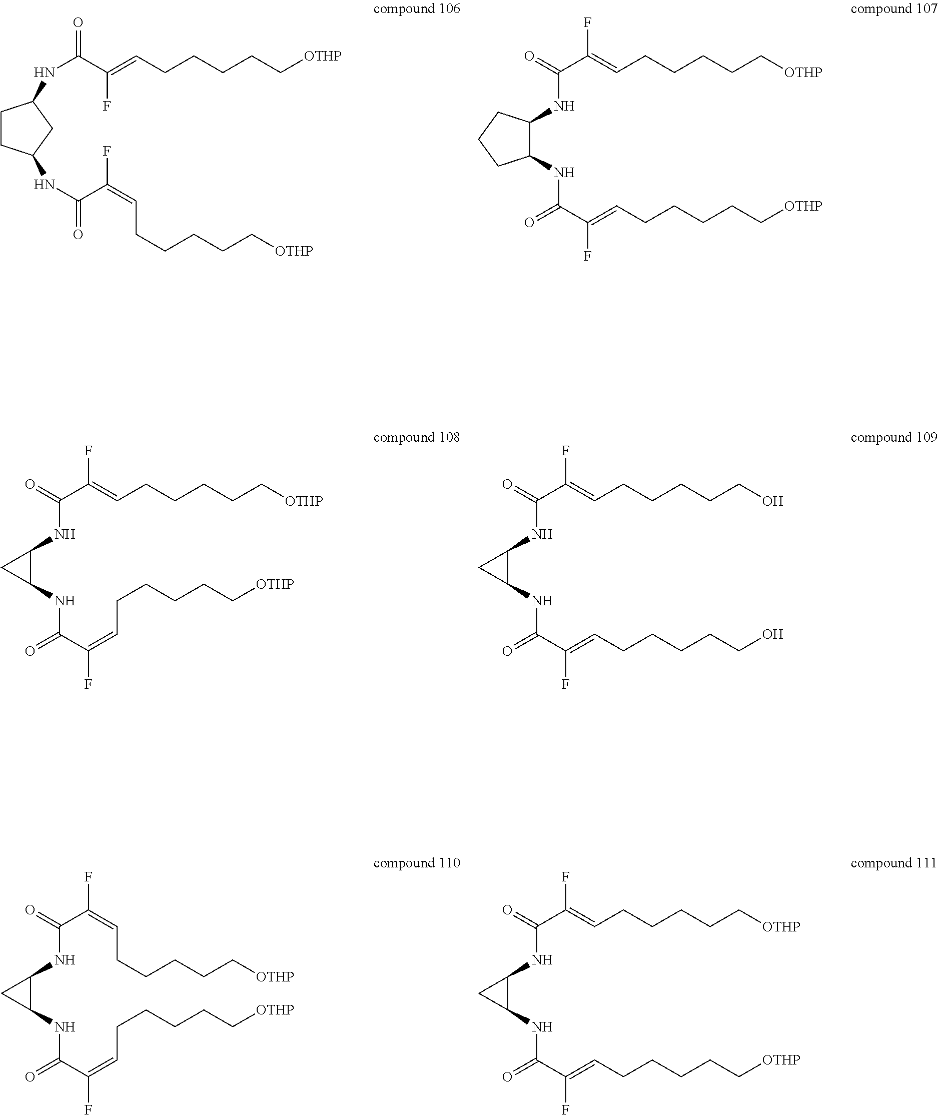

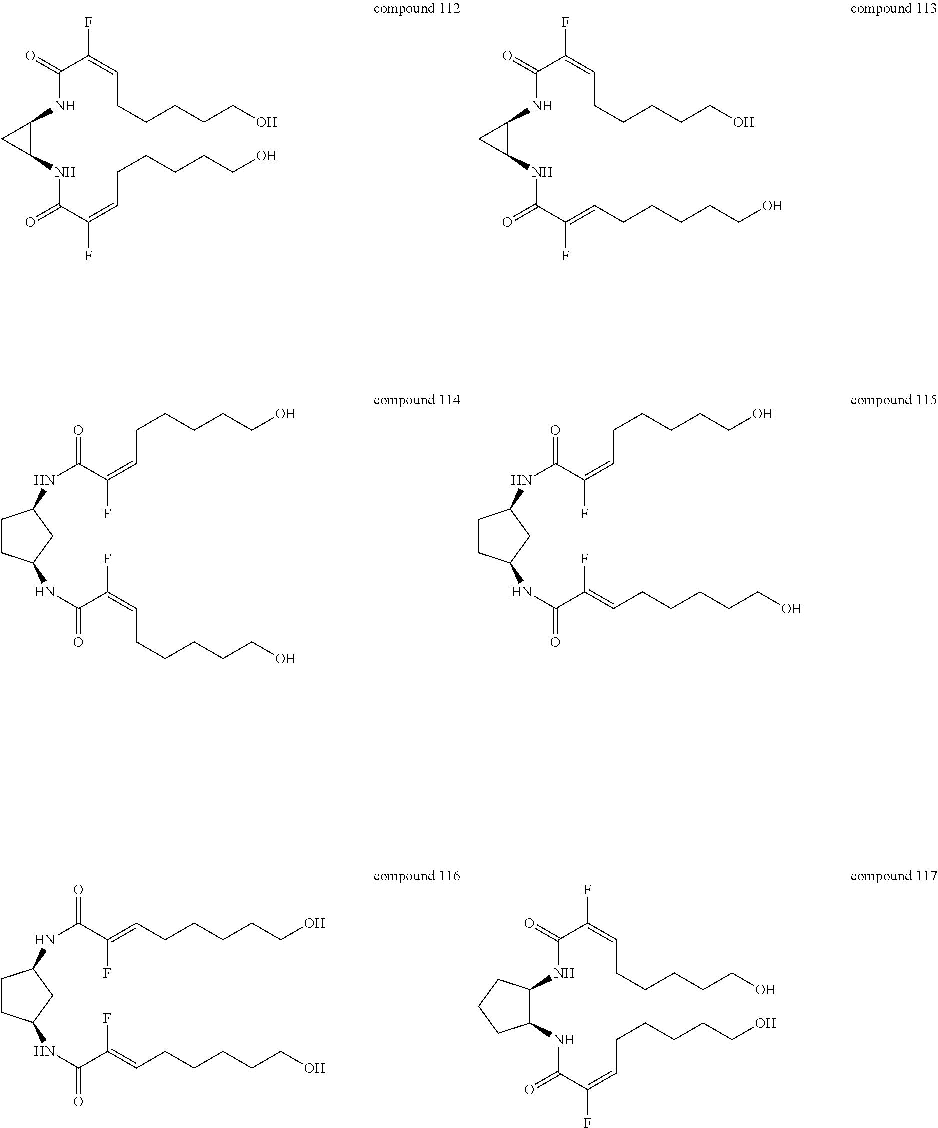

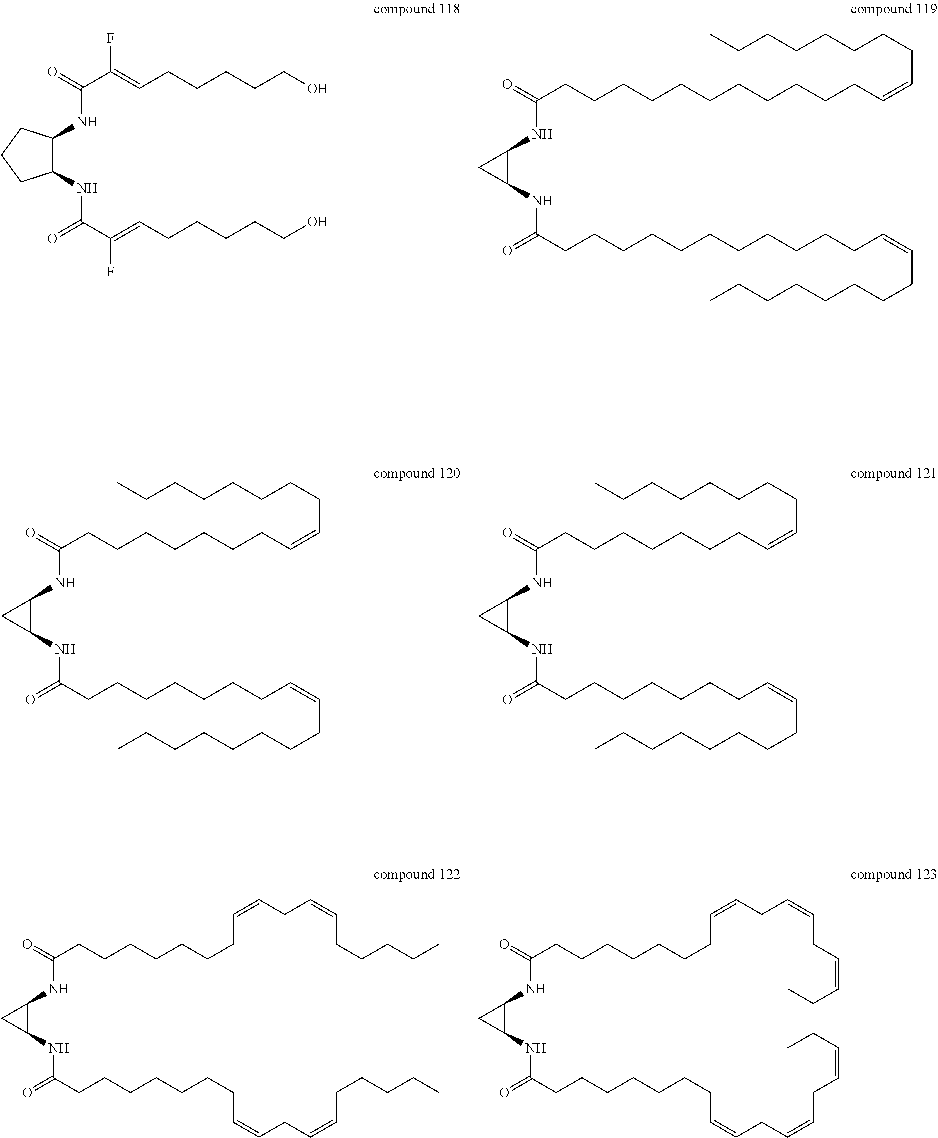

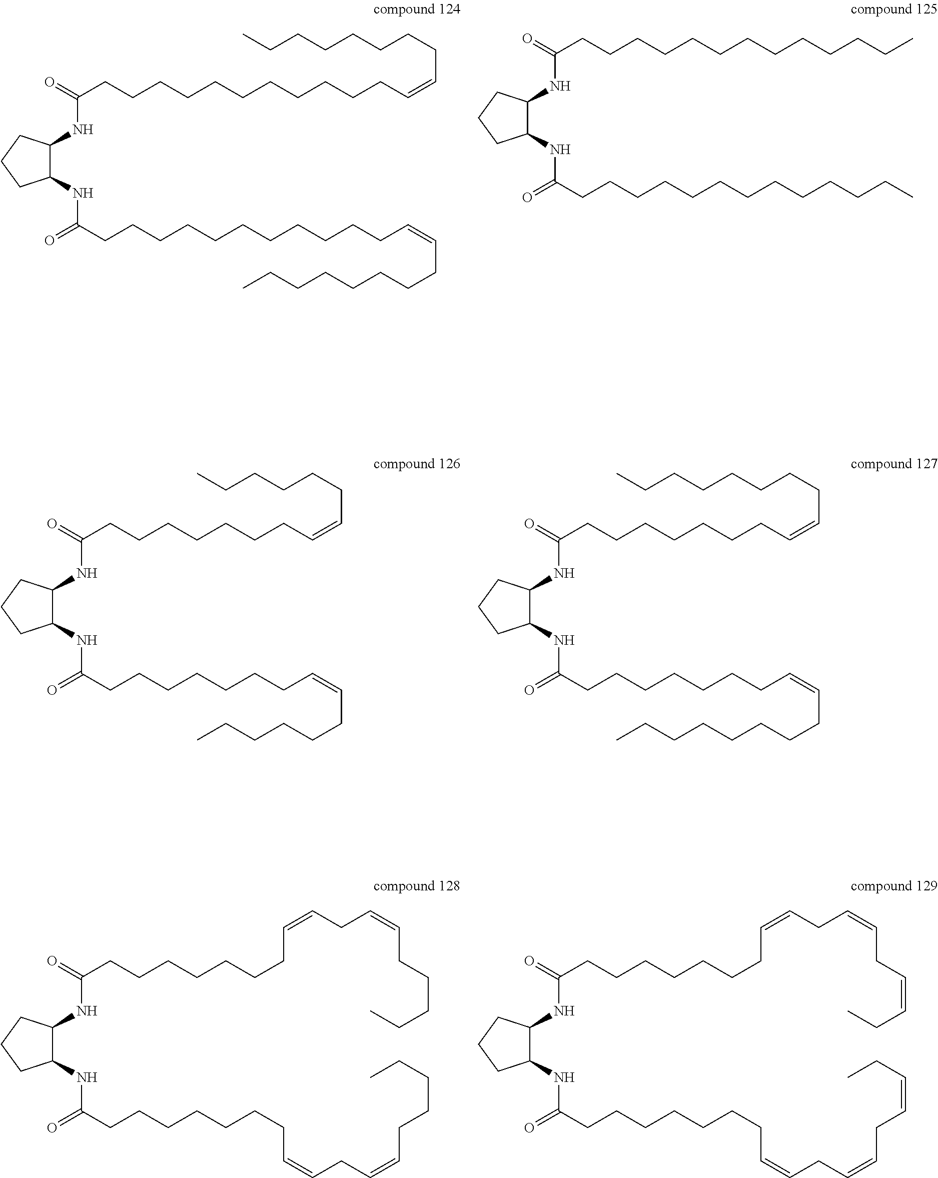

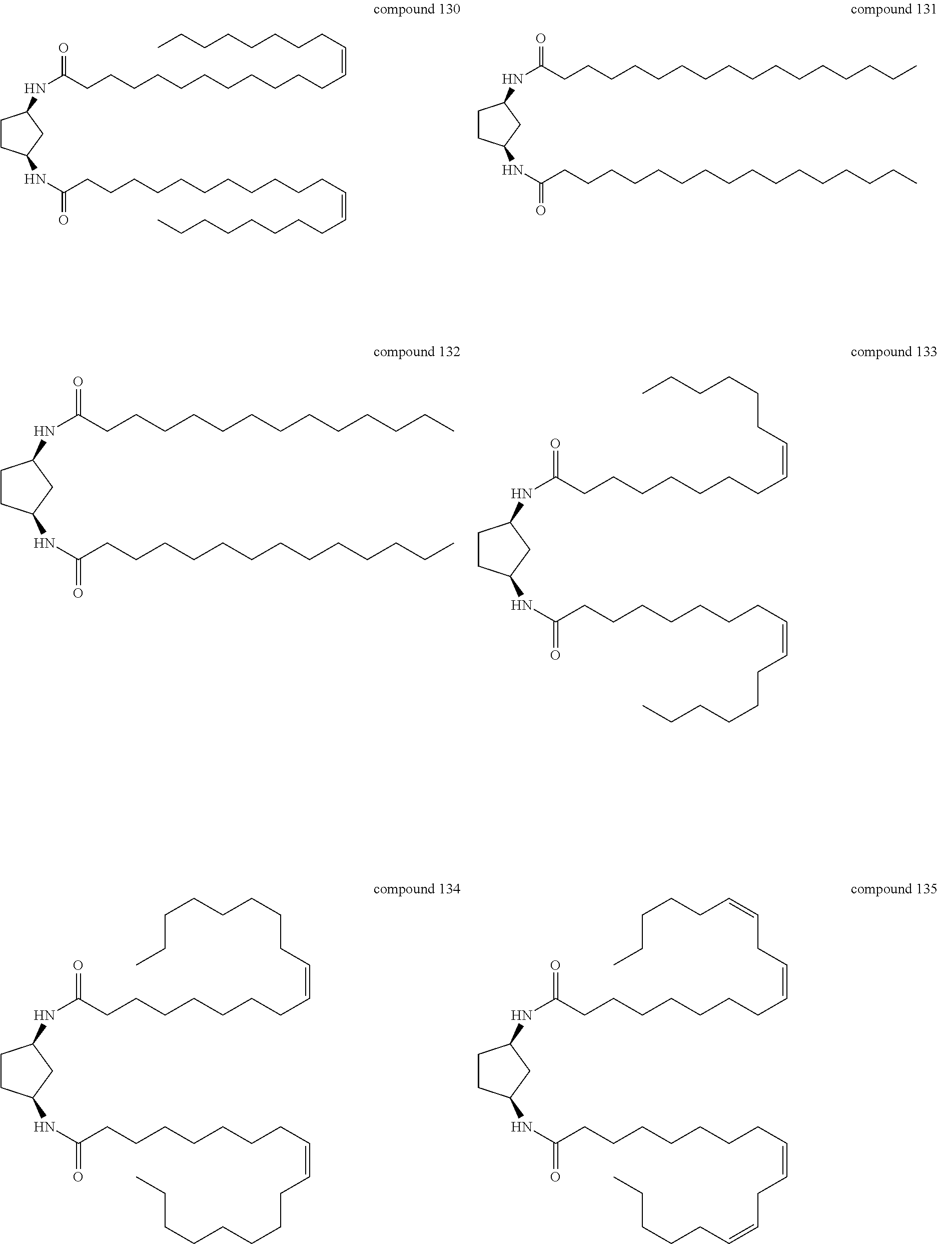

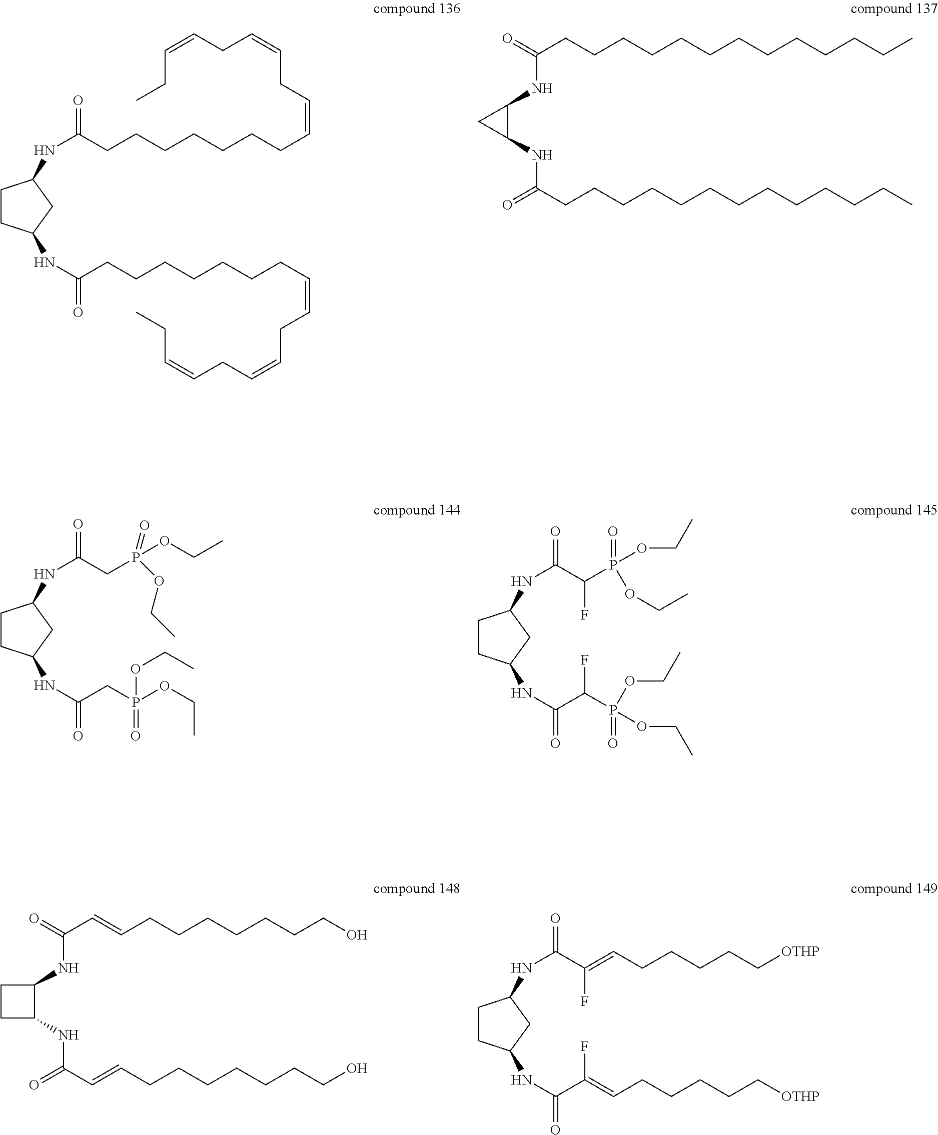

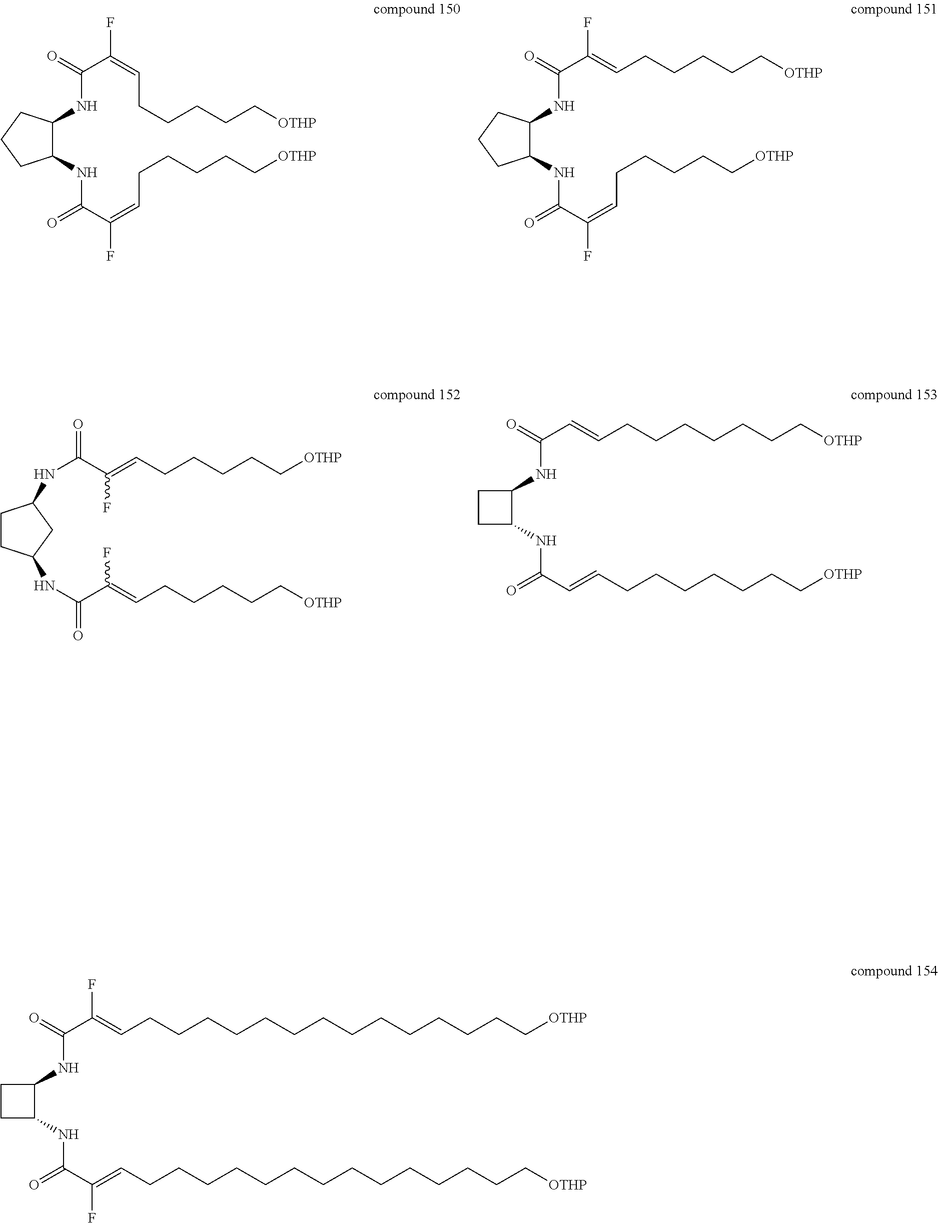

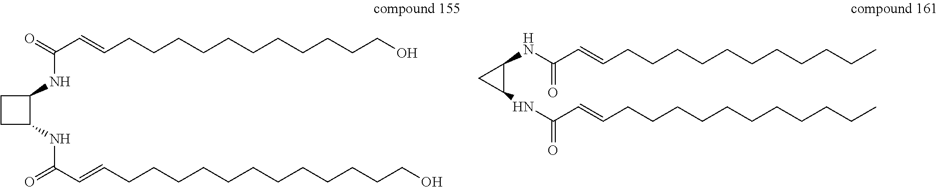

9. Process for the preparation of compounds according to claim 1, of Formula I, cis and trans, shown below: 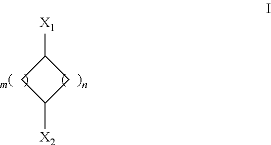
in which
m=1, 2, 3 and n=0, 1,
provided that m+n is different from 4,
X₁ and X₂ can be trans or cis relative to one another and represent, independently of one another, a group selected from 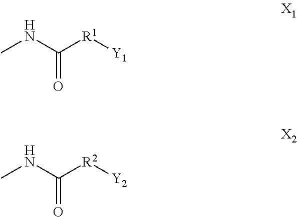
in which [symbol]
Y₁ represents
—H,
—OH,
—OH optionally coupled to a glycoside compound, which can be an α- or β-furanose or an α- or β-pyranose,
—ORₐ,
—OCOCH₃,
—OSi(Rₐ)₃,
—OSitBdPh of formula 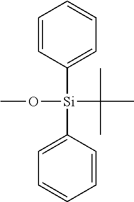 [SMILES: CO[Si](c1ccccc1)(c1ccccc1)C(C)(C)C]
—OSitBdM of formula 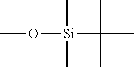
—COOH,
—COOR_{b},
—NH₂.
—NR_{c}R_{d},
—NHCORₑ,
—NHCOOR_{f},
the —OTHP group of formula, 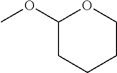
a group derived from ethylene glycol of formula, 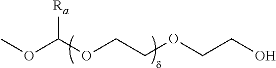
in which δ varies from 1 to 12,
a group derived from propylene glycol of formula, 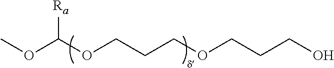
in which δ′ varies from 1 to 5,
an —O—CH(R_{z})—O-Q group, in which R_{z}, represents an alkyl or aralkyl group having from 1 to 30 carbon atoms, which can, but does not necessarily, contain one or more ether functions and optionally a terminal hydroxyl,
Rₐ, R_{b}, R_{c}, R_{d} represent linear or branched alkyl groups comprising from 1 to 4 carbon atoms, optionally substituted by one or more halogen atoms, or carbon chains interrupted by oxygen or sulphur atoms, benzyl groups optionally substituted by a halogen atom, a hydroxy group, an alkoxy group having from 1 to 8 carbon atoms,
Rₑ represents a linear or branched alkyl group containing from 1 to 4 carbon atoms, a phthalimido group (in this case the NH is replaced by N), a benzyl group optionally substituted by a halogen atom, a hydroxy group, an alkoxy group and in particular substituted by the methoxy group in the para position,
R_{f} represents a linear or branched alkyl group comprising from 1 to 4 carbon atoms, optionally substituted by one or more halogen atoms, or a carbon chain interrupted by oxygen or sulphur atoms, a phenyl group, a benzyl group optionally substituted by a halogen atom, a hydroxy group, an alkoxy group and in particular substituted by the methoxy group in the para position,
the phosphonate group of formula 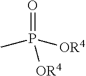
in which
R⁴ represents a linear or branched alkyl group, comprising from 1 to 6 carbon atoms, in particular methyl, ethyl, isopropyl, tert-butyl, (OR⁴)₂ optionally forming a ring between the two oxygen atoms, the (OR⁴)₂ groups in particular originating from diols such as ethane-1,2-diol, propane-1,3-diol, 2,2-dimethylpropane-1,3-diol, 2,3-dimethylbutane-2,3-diol (pinacol), 2-methylbutane-2,3-diol, 1,2-diphenylethane-1,2-diol, 2-methylpentane-2,4-diol, 1,2-dihydroxybenzene (catechol), 2,2′-azanediyldiethanol, 2,2′-(butylazanediyl)diethanol, 2,3-dihydroxysuccinic acid (tartaric acid) and its esters, or (OR⁴)₂ originates in particular from diacids such as 2,2′-(methylazanediyl)diacetic acid (mida), [symbol]
R¹ represents a linear or branched chain having from 1 to 30 carbon atoms, R¹ being saturated or unsaturated, unsubstituted or substituted by a halogen atom,
and in the case of an unsaturation, the C═C double bond optionally being substituted by a fluorine, chlorine, or bromine atom or by a —CF₃ group,
and in the case when R⁴ only comprises a single carbon, it is selected from the groups of Formula “—CHV—”, in which V represents —H, —F, —Cl or —Br, Y₁ then being equal to the phosphonate group —P(O)(OR⁴)₂, R⁴ having the meaning given above,
said process comprising a reaction of amide formation between a compound of Formula VII 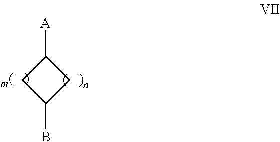
in which
m=1, 2, 3 and n=0, 1, provided that m+n is different from 4,
A=—NH₂, —NH—CO—R¹—Y₁
B=—NH₂, —NH—CO—R¹—Y₁
provided that if A=—NH—CO—R¹—Y₁ then B=—NH₂,
and a compound of general formula VIII 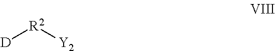
in which
Y₂ has the same meaning as Y₁,
R² has the same meaning as R¹,
Y₁ and Y₂ can be equal or different,
R¹ and R² can be equal or different,
D=—CO—R⁵
R⁵ representing
a hydroxy group —OH,
an alkoxy group —OR⁶, R⁶ representing a linear or branched alkyl chain comprising from 1 to 8 carbon atoms,
a chlorine atom —Cl,
an acyloxy group —O—CO—R⁷, R⁷ representing a linear or branched alkyl chain comprising from 1 to 8 carbon atoms, or optionally being equal to —R²—Y₂, the meanings of R² and of Y₂ being those defined above,
a group derived from benzotriazole —OR⁸, of formula 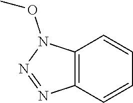
in particular derived from
HATU (2-(1H-7-azabenzotriazol-1-yl)-1,1,3,3-tetramethyluronium hexafluorophosphate methanaminium),
HBTU (2-(1H-benzotriazol-1-yl)-1,1,3,3-tetramethyluronium hexafluorophosphate,
HOBt (1-hydroxybenzotriazole),
BOP (benzotriazol-1-yl-oxy-tris-(dimethylamino)-phosphonium hexafluorophosphate),
PyBOP (benzotriazol-1-yl-oxy-tris-(dimethylamino)-phosphonium hexafluorophosphate),
a group derived from a carbodiimide, of formula 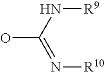
in which
R⁹ and R¹⁰, different or equal, represent an alkyl group comprising from 1 to 10 carbon atoms, linear, branched or cyclic, optionally substituted by an amino group, in particular cyclohexyl, isopropyl, ethyl, dimethylpropylamino,
said carbodiimide in particular being selected from the following compounds
DCC (N,N′-dicyclohexylcarbodiimide),
EDCI (1-ethyl-3-(3-dimethylaminopropyl) carbodiimide),
DIC (N,N′-diisopropylcarbodiimide),
said reaction of amide formation making it possible to obtain the compounds of Formula I shown above.

10. Process for the preparation according to claim 9, of the compounds of Formula I_{A} and I_{B}, cis and trans, represented by the formulae shown below: 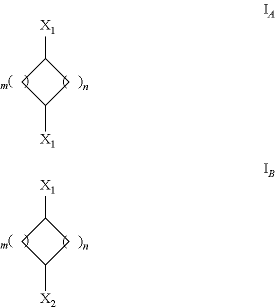
in which
X₁ and X₂ have the meanings as previously defined,
which comprises an amide formation between a compound of Formula VII shown below: 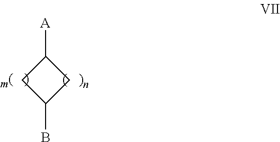
in which
m=1, 2, 3 and n=0, 1, provided that m+n is different from 4,
A and B are such that: [symbol]
A=B=—NH₂, [symbol]
or A=—NH₂ and B=—NH—CO—R¹—Y₁,
and a compound of Formula VIII_{A} 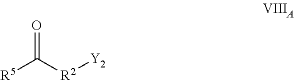
in which
R², R⁵ and Y₂ have the meanings as previously defined,
Y₁ and Y₂ can be equal or different,
R¹ and R² can be equal or different,
said process making it possible to obtain the compounds of Formulae I_{A} and I_{B} represented above,
and in particular of symmetrical compounds of Formula II_{A} cis and trans shown below: 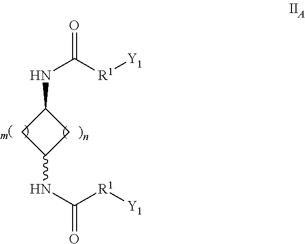
in which
m=1, 2, 3 and n=0.1, provided that m+n is different from 4,
R¹, Y₁ have the meanings as previously defined,
comprising coupling between a cis or trans diamine of Formula VII_{A} shown below: 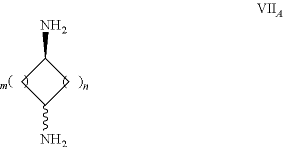
in which
m and n have the meanings given above,
and a compound of Formula VIII_{A} 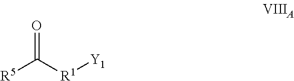
R¹, R⁵ and Y₁ having the meanings given above,
R⁵ having the meanings as previously defined, said process making it possible to obtain the compounds of Formula II_{A} represented above,
and in particular of symmetrical compounds of Formula II_{A cis} represented below 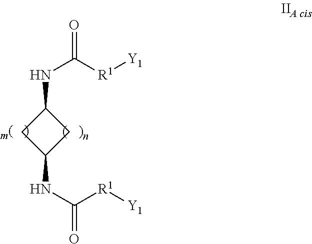
in which
m=1, 2, 3 and n=0.1, provided that m+n is different from 4,
R¹ and Y₁ have the meanings as previously defined,
said process comprising coupling between a diamine of Formula VII_{A cis} shown below: 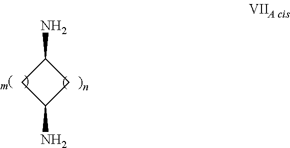
in which
m and n have the meanings given above,
and a compound of Formula VIII_{A} 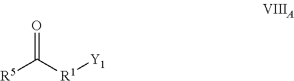
R⁵ having the meanings as previously defined and in particular being equal to —OH,
R¹ and Y₁ having the meanings given above,
said process making it possible to obtain the compounds of Formula II_{A cis} represented above.

11. Process for the preparation according to claim 1, of the symmetrical compounds of Formula VI_{F cis} represented below 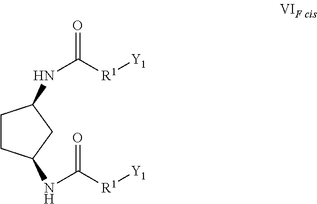
in which
R¹ and Y₁ have the meanings as previously defined,
said process comprising coupling between cis-1,3-diaminocyclopentane of the formula shown below 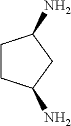
and a compound of Formula VIII_{A} 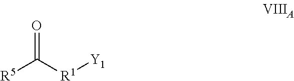
R⁵ having the meanings as previously defined and in particular being equal to —OH,
R¹ and Y₁ having the meanings given above,
said process making it possible to obtain the compounds of Formula VI_{F cis} represented above,
and in particular of compound 30 of the formula shown below 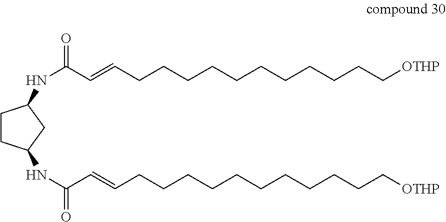
in which —OTHP has the meaning as previously defined,
said process comprising coupling between cis-1,3-diaminocyclopentane of the formula shown below 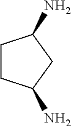
and the acid of the formula shown below 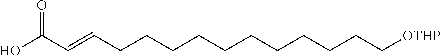
in which —OTHP has the meaning designated above, said process making it possible to obtain compound 30 of the formula shown above,
and in particular compound 152 of the formula shown below 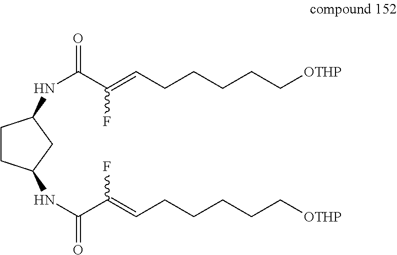
in which —OTHP has the meaning as previously defined,
said process comprising coupling between cis-1,3-diaminocyclopentane of the formula shown below: 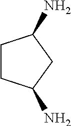
and the acid of the formula shown below 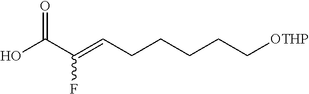
in which —OTHP has the meaning designated above,
said process making it possible to obtain compound 152 of the formula shown above.

12. Process for the preparation according to claim 9, of the cis and trans asymmetrical compounds of Formula II_{B} represented below 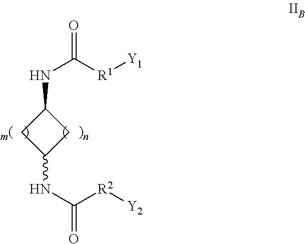
in which
m=1, 2, 3 and n=0, 1, provided that m+n is different from 4,
R¹, R², Y₁ and Y₂ have the meanings as previously defined,
provided that R¹ and R² are different from one another,
Y₁ and Y₂ are identical or different,
which comprises a reaction between an aminoamide of Formula VII_{D} represented by the formula shown below: 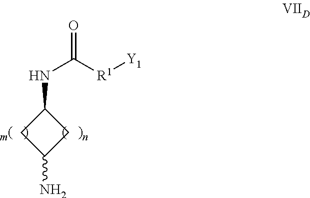
in which
R¹, Y₁, m and n have the meanings given above,
and a compound of Formula VIII_{A} 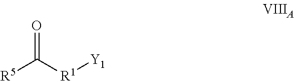
in which
R², R⁵ and Y₂ have the meanings as previously defined,
said process making it possible to obtain the compounds of Formula II_{B} represented above,
and in particular in which compound VII_{D} represented by the formula shown below 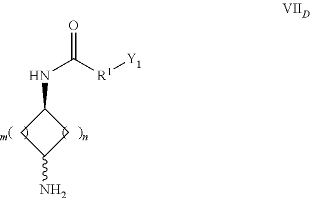
is obtained by deprotection of the amine function of compound IX represented below 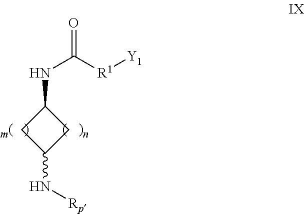
in which
Rp′ is a protective group of the amines selected from:
—CORₑ, in which Rₑ represents a linear or branched alkyl group containing from 1 to 4 carbon atoms, a phthalimido group (in this case the NH is replaced by N), a benzyl group optionally substituted by a halogen atom, a hydroxy group, an alkoxy group and in particular substituted by the methoxy group in the para position,
—COOR_{f}, in which R_{f} represents a linear or branched alkyl group comprising from 1 to 4 carbon atoms, optionally substituted by one or more halogen atoms, more particularly methyl, ethyl, propyl, tert-butyl, or a carbon chain interrupted by oxygen or sulphur atoms, a phenyl group, a benzyl group or derivatives thereof, optionally substituted by a halogen atom, a hydroxy group, an alkoxy group and in particular substituted by the methoxy group in the para position,
the benzyl group or derivatives thereof,
m=1, 2, 3 and n=0, 1, provided that m+n is different from 4,
R¹, Y₁ have the meanings as previously defined,
said process making it possible to obtain the compounds of Formula VII_{D} represented above,
and in particular in which compound IX represented by the formula shown below 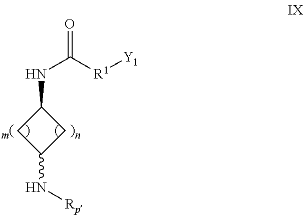
is obtained by monoacylation between the diamine X, one amine function of which is blocked by a protective group 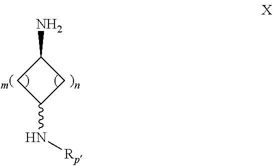
in which
Rp′ is a protective group of the amines selected from:
—CORₑ, in which Rₑ represents a linear or branched alkyl group containing from 1 to 4 carbon atoms, a phthalimido group (in this case the NH is replaced by N), a benzyl group optionally substituted by a halogen atom, a hydroxy group, an alkoxy group and in particular substituted by the methoxy group in the para position,
—COOR_{f}, in which R_{f} represents a linear or branched alkyl group comprising from 1 to 4 carbon atoms, optionally substituted by one or more halogen atoms, more particularly methyl, ethyl, propyl, tert-butyl, or a carbon chain interrupted by oxygen or sulphur atoms, a phenyl group, a benzyl group or derivatives thereof, optionally substituted by a halogen atom, a hydroxy group, an alkoxy group and in particular substituted by the methoxy group in the para position,
the benzyl group or derivatives thereof,
m=1, 2, 3 and n=0, 1, provided that m+n is different from 4,
R¹, Y₁ have the meanings as previously defined,
and a compound of Formula VIII_{A} represented by the formula shown below: 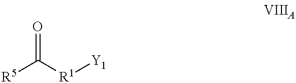
in which
R¹, R⁵ and Y₁ have the meanings as previously defined,
said process making it possible to obtain the compounds of Formula IX represented above,
and in particular in which compound X represented by the formula shown below 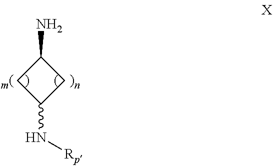
is obtained by protection of the diamine of Formula VII_{A} shown below: 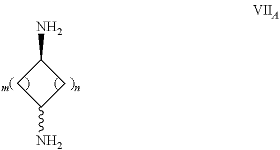
in which
m=1, 2, 3 and n=0, 1, provided that m+n is different from 4, said process making it possible to obtain the compounds of Formula X represented above.

13. Preparation process according to claim 9, in which the compounds of Formula II_{C} shown below: 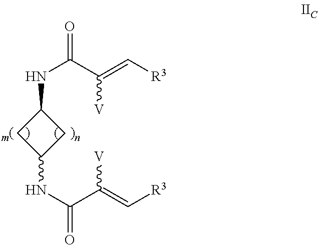
in which
m=1, 2, 3 and n=0, 1, provided that m+n is different from 4,
V=H, F, Cl or Br,
R³ represents an unbranched linear alkyl chain, saturated or unsaturated, comprising from 5 to 28 carbon atoms, terminated by a hydrogen, an —OH group, or a protected form of the latter, an —NH₂ group or a protected form of the latter, in particular —NHBoc,
said compounds II_{C} being obtained by Wittig-Horner reaction between an aldehyde of general formula XVII 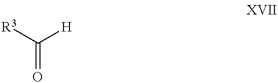
R₃ having the meaning given above,
and a phosphonoacetamide of general formula XVIII 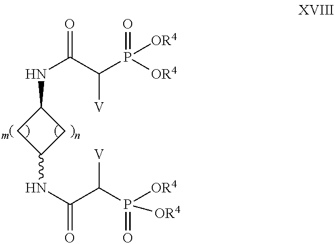
in which
m, n and V have the meanings given above,
R⁴ represents a linear or branched alkyl group, comprising from 1 to 6 carbon atoms, in particular methyl, ethyl, isopropyl, tert-butyl, (OR⁴)₂ optionally forming a ring between the two oxygen atoms, the (OR⁴)₂ groups in particular originating from diols such as ethane-1,2-diol, propane-1,3-diol, 2,2-dimethylpropane-1,3-diol, 2,3-dimethylbutane-2,3-diol (pinacol), 2-methylbutane-2,3-diol, 1,2-diphenylethane-1,2-diol, 2-methylpentane-2,4-diol, 1,2-dihydroxybenzene (catechol), 2,2′-azanediyldiethanol, 2,2′-(butylazanediyl)diethanol, 2,3-dihydroxysuccinic acid (tartaric acid) and its esters, or (OR⁴)₂ originates in particular from diacids such as 2,2′-(methylazanediyl)diacetic acid (mida), in particular methyl, ethyl, isopropyl, tert-butyl,
said process making it possible to obtain the compounds of Formula II_{C} represented above.

14. Preparation process according to claim 9, in which the phosphonamide XVIII represented by the formula shown below: 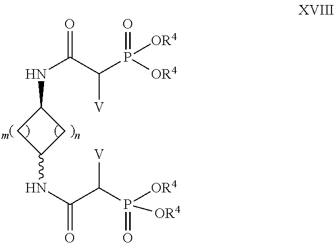
in which
m=1, 2, 3 and n=0, 1, provided that m+n is different from 4,
V=H, F, Cl or Br,
R⁴ represents a linear or branched alkyl group, comprising from 1 to 6 carbon atoms, in particular methyl, ethyl, isopropyl, tert-butyl, (OR⁴)₂ optionally forming a ring between the two oxygen atoms, the (OR⁴)₂ groups in particular originating from diols such as ethane-1,2-diol, propane-1,3-diol, 2,2-dimethylpropane-1,3-diol, 2,3-dimethylbutane-2,3-diol (pinacol), 2-methylbutane-2,3-diol, 1,2-diphenylethane-1,2-diol, 2-methylpentane-2,4-diol, 1,2-dihydroxybenzene (catechol), 2,2′-azanediyldiethanol, 2,2′-(butylazanediyl)diethanol, 2,3-dihydroxysuccinic acid (tartaric acid) and its esters, or (OR⁴)₂ originates in particular from diacids such as 2,2′-(methylazanediyl)diacetic acid (mida), in particular methyl, ethyl, isopropyl, tert-butyl,
said compound XVIII being obtained by an amide formation between the diamine of general formula VII_{A} shown below: 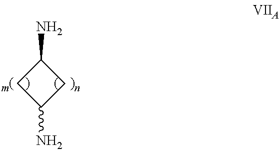
m and n having the meanings given above,
and the phosphorylated carboxylic acid of Formula VIII_{C} 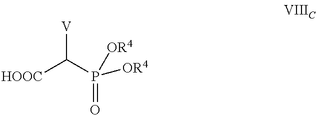
in which
V and R⁴ have the meanings given above,
said process making it possible to obtain the compounds of Formula XVIII represented above.

15. Pharmaceutical composition containing, as active ingredient, at least one of the compounds mentioned in claim 1, and in particular containing, as active ingredient, compound 30 of formula 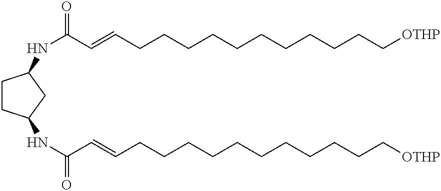
and/or compound 152 of formula 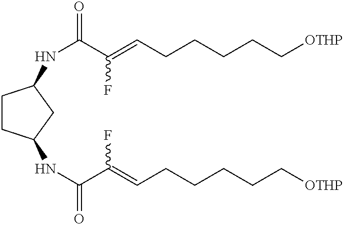
in combination with a pharmaceutically acceptable vehicle.

16. Pharmaceutical composition containing, as active ingredient, several of the compounds mentioned in claim 1, and in particular containing, as active ingredient, several compounds including compound 30 and/or compound 152
in combination with a pharmaceutically acceptable vehicle.

17. Cosmetic composition containing, as active ingredient, at least one of the compounds mentioned in claim 1, and in particular containing, as active ingredient, several compounds including compound 30 of formula 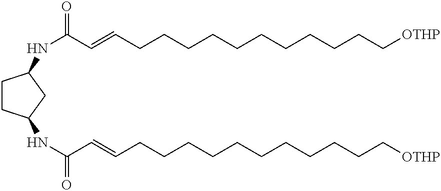
and/or compound 152 of formula 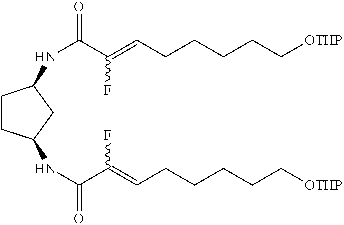
in combination with a cosmetically acceptable vehicle.

18. Cosmetic composition containing, as active ingredient, several of the compounds mentioned in claim 1, and in particular containing, as active ingredient, several compounds including compound 30 and/or compound 152, in combination with a cosmetically acceptable vehicle.
